(19) United States
(12) Patent Application Publication
(10) Pub. No.: US 2023/0194524 A1
(43) Pub. Date: Jun. 22, 2023

(54) Stem Cell Memory T Cells, Methods and Uses for Modulating Inflammatory Responses, and Diagnosing and Treating Adverse Cardiovascular Events and Disease

(71) Applicant: La Jolla Institute for Immunology, La Jolla, CA (US)

(72) Inventors: Catherine Lynn Hedrick, La Jolla, CA (US); Lindsey Padgett, La Jolla, CA (US)

(21) Appl. No.: 17/776,417

(22) PCT Filed: Nov. 16, 2020
(86) PCT No.: PCT/US2020/060770

§ 371 Date: May 12, 2022

Related U.S. Application Data
(60) Provisional application No. 62/936,485, filed on Nov. 16, 2019.

(51) Int. Cl. G01N 33/569; G01N 33/68

(52) U.S. Cl. CPC G01N 33/56972; G01N 33/6863; G01N 2333/705; G01N 2800/32; G01N 2800/52

(57) ABSTRACT

A method of diagnosing and treating a cardiovascular disease in a patient, the method comprising the steps of: determining whether the patient has an increase in stem cell memory T (T_{scm}) cells by: obtaining or having obtained a biological sample from the patient; performing or having performed an assay on the biological sample to determine the amount of T_{scm} in the patient, wherein the T scm are at least one of: CD8+CD45RA+CCR7+CD27+CD28+CD95+ T cells, CD4+CD45RA+CCR7+CD27+CD28+CD95+ T cells, or CD8+CD3+CD45RA+CCR7+C D45RO− CD95+ T cells; identifying that the patient has an increase in T_{scm} when compared to the amount of T_{scm} in a healthy patient; and if the patient has an increase in T_{scm}, then administering therapy for the treatment of the cardiovascular disease to the patient.

### GOVERNMENT SUPPORT

This invention was made with government support under contract/grant numbers R01 HL134236, P01 HL136275, P01 HL005798, and R01 CA202987 awarded by NIH. The Government has certain rights in the invention.

### FIELD OF THE INVENTION

The invention relates to the field of treatment and diagnosis of cardiovascular diseases and events, including severe cardiovascular disease, via the identification and quantification of stem cell memory T cells and levels thereof, including when compared to the level of naïve T cells, in a subject.

### BACKGROUND

Cardiovascular disease (CVD) represents a major health concern and accounts for nearly one third of all mortalities world-wide despite the development of statins¹. Within atherosclerosis, the main underlying trigger of CVD, activated CD4⁺ and CD8⁺ T cells infiltrate atherosclerotic plaques, leading to destabilization, rupture, and myocardial infarction. The importance of CD4⁺ T cells in atherosclerosis has been well documented, with pro-inflammatory T helper 1 (Th1) CD4⁺ T cells promoting atherosclerosis via secretion of the pro-inflammatory cytokine IFN-γ, while immunoregulatory CD4⁺ regulatory T cells (Tregs) protect against atherosclerosis by dampening autoreactive immune responses via the immunosuppressive cytokines IL-10 and TGF-β²⁻⁵.

In direct contrast to CD4⁺ T cells, much less is known about how CD8⁺ T cells contribute to atherogenesis. Numerous studies have cited a pro-atherogenic role for CD8⁺ T cells in mice. Specifically, depletion of CD8⁺ T cells utilizing monoclonal antibodies significantly reduced atherosclerotic lesions in atherosclerotic Apolipoprotein E-deficient (ApoE^{-/-}) mice; conversely, CD8⁺ T cell adoptive transfer heightened atherosclerosis⁶. CD137 (4-1B) depletion resulted in an influx of CD8⁺ T cells within the atherosclerotic plaque, with an accompanying increase in atherosclerosis disease severity 7. Feeding mice a high fat diet elicited an influx of IFN-γ-expressing CD8⁺CD28⁺ T cells within the aortic root-draining mediastinal lymph nodes at four weeks, whereas activated CD4+ T cells were not observed until 8 weeks of high fat diet feeding^{7,8}.

While numerous studies have cited a pro-atherogenic role for CD8⁺ T cells, several reports have demonstrated that CD8⁺ T cells play a non-essential role or a protective role in atherosclerosis. In particular, depletion of the gene CD8a within ApoE^{-/-} mice elicited unchanged atherosclerotic lesions, in contrast to depletion of total TCR-β T cells or CD4⁺ T cells 9. Deficiency in transporter antigen processing-1 (TAP-1), required for MHC-I antigen presentation, elicited drastically attenuated CD8⁺ T cell numbers but did not alter atherosclerosis¹⁰. Immunization of ApoE^{-/-} mice with p210, a peptide derived from ApoB100 that selectively expands CD8⁺ T cells, elicited reduced atherosclerotic lesions in comparison to control animals, indicating a protective role of CD8⁺ T cells. Moreover, CD8⁺CD25⁺ T cells, noted by increased expression of the immunosuppressive molecules CTLA-4, Foxp3, and TGF-β, were identified in western diet-fed ApoE^{-/-} mice and suppressed proliferation and cytotoxic functions of effector T cells. With contrasting pro-inflammatory and protective roles in atherosclerosis, further study is warranted to determine the role of CD8⁺ T cells in atherosclerosis.

Despite the controversy surrounding the role and importance of CD8⁺ T cells in atherosclerosis, multiple lines of evidence indicate that CD8⁺ T cells play a prominent role in clinical atherosclerosis and CVD¹¹⁻¹³ Specifically, CD8⁺ T cells constitute a substantial portion of the atherosclerotic plaque infiltrate of CVD individuals, localized predominantly within the shoulder regions and fibrous caps^{11,12,14} In early disease stages within humans, CD8⁺ T cells within the vascular intima constituted 29% of all leukocytes and increased to approximately 50% in advanced atherosclerotic plaques, with cytotoxic CD8⁺ T cells the dominant infiltrating population¹¹. Of note, plaque infiltrating CD8⁺ T cells were more highly activated compared to plaque- or blood-isolated CD4⁺ T cells^{11,15} Thus, CD8⁺ T cells constitute a dominant presence within the atherosclerotic plaque.

In addition to the plaque, circulating CD8⁺ T cell memory phenotypes positively correlate with cardiovascular disease. Elevated circulating highly cytotoxic CD8⁺ T cells expressing CD56 and CD57 were observed in CVD individual peripheral blood. Enhanced CD8⁺CD57⁺ T cell frequencies were linked to short-term cardiovascular mortality in acute myocardial infarction (MI)^{16,17}. Effector memory CD8⁺ T cells with low expression of the IL-6R were expanded with cardiovascular disease¹⁸.

The inventors sought to examine further how T cell signatures correlated with human CVD risk at the single cell level and performed high-dimensional mass cytometry on 28 matched individuals with low and severe (high) CVD risk.

### SUMMARY

The inventors examined T cell signatures that correlate with high risk of myocardial infarction or heart attack (high CVD) and pinpointed a naïve CD8+ T cell (T_{N}) expressing CD95 that was robustly increased compared to individuals with a low risk (low CVD). The inventors discovered these CD8+ T cells express markers of stem cell memory (T_{SCM}) cells and are pro-atherogenic and a biomarker of CVD risk. Further, a population of CD8 T_{CD25} was also identified to be atheroprotective and/or a biomarker of CVD risk.

In one embodiment, the present invention includes a method of diagnosing and treating a cardiovascular disease in a patient, the method comprising the steps of: determining whether the patient has an increase in stem cell memory T cells (T_{scm}) by: obtaining or having obtained a biological sample from the patient; performing or having performed an assay on the biological sample to determine the amount of T_{scm} in the patient, wherein the T_{scm} are at least one of: T_{scm} CD8+CD45RA+CCR7+CD27+CD28+CD95+ T cells, T_{scm} CD4+CD45RA+CCR7+CD27+CD28+CD95+ T cells, or T_{scm} CD8+CD3+CD45RA+CCR7+CD45RO−CD95+ T cells, identifying that the patient has an increase in T_{scm} when compared to the amount of T_{scm} in a healthy patient; and if the patient has an increase in T_{scm}, then administering therapy for the treatment of the cardiovascular disease to the patient; and if the patient has an increase in T_{scm}, then administering therapy for the treatment of the cardiovascular disease to the patient. In one aspect, the diagnosing comprises determining the amount of increase in T_{scm} of the patient compared to the amount of T_{scm} in a healthy patient to identify whether the patient has cardiovascular disease, is at risk of developing cardiovascular disease, or has advanced or severe cardiovascular disease. In another aspect, the increase in T_{scm} when compared to the amount of T_{scm} in a healthy patient comprises determining the ratio of T_{scm} to naïve T (T_{N}) cells in the patient when compared to the ratio of T_{scm} to Tₙ cells in the healthy patient, wherein the T_{N} are CD8+CD3+CD45RA+CCR7+CD45RO−CD95− T cells, and wherein a higher ratio of T_{scm} to TN in the patient indicates the patient has a greater risk of cardiovascular disease or more advanced cardiovascular disease. In another aspect, a ratio of 1.5:1 T_{scm} to Tₙ cells or greater compared to the ratio of T_{scm} to Tₙ cells in the healthy patient indicates the patient has a cardiovascular disease. In another aspect, the amount of T_{scm} is determined by detecting the amount of T_{scm} in a blood sample. In another aspect, the amounts of T_{scm} and Tₙ are determined by detecting the amounts T_{scm} and Tₙ in a blood sample. In another aspect, wherein the T_{scm} or the T_{N} express one or more of the following markers: CD127, CD11a, CD58, IL-2Rβ, or Bcl-2. In another aspect, the blood sample comprises peripheral blood mononuclear cells (PBMCs). In another aspect, the cardiovascular disease is severe. In another aspect, the T_{scm} express one or more of the following markers: CXCR3, CD122, LFA-1, c-Myb, Ki67, CD127, CD11a, CD58, IL-2Rβ, or Bcl-2. In another aspect, the increase of T_{scm} in the patient is further determined by measuring an increased expression of IFN-γ in T_{scm} of the patient compared to the expression of IFN-γ in T_{scm} of a healthy patient. In another aspect, the therapy for the treatment of the cardiovascular disease comprises a T_{scm}, T_{N}, or T_{CD25} modulating therapy. In another aspect, the T_{scm}, T_{N}, or T_{CD25} modulating therapy is a T_{scm} depleting therapy, a T_{N} increasing therapy or a T_{CD25} CD8+CD3+CD25+ T cell therapy. In another aspect, the therapy for the treatment of the cardiovascular disease is administered systemically, regionally or locally, via ingestion, via inhalation, topically, intravenously, or orally.

In another embodiment, the present invention includes a method for treating a patient suffering from cardiovascular disease, the method comprising the steps of: determining whether the patient has an increase in stem cell memory T (T_{scm}) cells, when compared to a baseline level of T_{scm} present in a healthy patient by: obtaining or having obtained a biological sample from the patient; and performing or having performed an assay on the biological sample to determine if the patient has an increase in T_{scm} when compared to a baseline level of T_{scm} present in healthy patient; and if the patient has an increase in T_{scm} when compared to a baseline level of T_{scm} present in a healthy patient, then administering therapy for the treatment of the cardiovascular disease to the patient. In one aspect, the increase in T_{scm} when compared to the baseline level of T_{scm} in a healthy patient comprises determining the ratio of T_{scm} to naïve T (T_{N}) cells in the patient when compared to the ratio of T_{scm} to TN cells in the healthy patient, wherein the TN are CD8+CD45RA+CCR7+CD27+CD28+ T cells, CD4+CD45RA+CCR7+CD27+CD28+ T cells, or T_{scm} CD8+CD3+CD45RA+CCR7+CD45RO−CD95+ T cells, and wherein at least one of: a higher ratio of T_{scm} to T_{N} in the patient indicates the patient has cardiovascular disease, is at risk of developing a cardiovascular disease, or has advanced cardiovascular disease; a patient with a high risk of myocardial infarction or heart attack (high CVD) has an increase in CD8+ T_{SCM} frequency (% of CD8+ T_{N} cells that are CD8+ T_{SCM} cells) when compared to a patient with a low risk of myocardial infarction or heart attack (low CVD); a patient with a high risk of myocardial infarction or heart attack (high CVD) has an increase of CD8+ T_{SCM} compared to subjects with a low risk of myocardial infarction or heart attack (low CVD); a patient with a high risk of myocardial infarction or heart attack (high CVD) have a decrease in CD8+ TN compared to subjects with a low risk of myocardial infarction or heart attack (low CVD); CD8+ T_{SCM} from subjects with a high risk of myocardial infarction or heart attack (high CVD) have higher expression and release of pro-atherogenic cytokine IFN-γ compared to CD8+ T_{SCM} from subjects with low CVD; or CD8+ T_{SCM} from patients with a high risk of myocardial infarction or heart attack (high CVD) have a higher expression of Ki67 compared to CD8+ T_{scm} from subjects with low CVD. In another aspect, a ratio of 1.5:1 T_{scm} to Tₙ cells or greater compared to the ratio of T_{scm} to Tₙ cells in the healthy patient indicates the patient should be treated for the cardiovascular disease. In another aspect, the increase in T_{scm} is determined by detecting T_{scm} in a blood sample. In another aspect, the ratio of T_{scm} and Tₙ are determined by detecting T_{scm} and Tₙ in a blood sample. In another aspect, the TN express one or more of the following markers: CD127, CD11a, CD58, IL-2Rβ, or Bcl-2. In another aspect, the blood sample comprises peripheral blood mononuclear cells (PBMCs). In another aspect, the cardiovascular disease is severe. In another aspect, the T_{scm} at least one of: T_{scm} CD8+CD45RA+CCR7+CD27+CD28+CD95+ T cells, T_{scm} CD4+CD45RA+CCR7+CD27+CD28+CD95+ T cells, or T_{scm} CD8+CD3+CD45RA+CCR7+CD45RO−CD95+ T cells. In another aspect, the T_{scm} express one or more of the following markers: CXCR3, CD122, LFA-1, c-Myb, Ki67, CD127, CD11a, CD58, IL-2RD, or Bcl-2. In another aspect, the increase of T_{scm} in the patient is further determined by measuring an increased expression of IFN-γ in T_{scm} of the patient compared to the expression of IFN-γ in T_{scm} of a healthy patient. In another aspect, the therapy for the treatment of cardiovascular disease comprises a T_{scm}, T_{N}, or T_{CD25} modulating therapy. In another aspect, the T_{scm}, T_{N}, or TCD25 modulating therapy is a T_{scm} depleting therapy, a Tₙ increasing therapy, or a TCD25 CD8+CD3+CD25+ T cell therapy. In another aspect, the therapy for the treatment of the cardiovascular disease is administered systemically, regionally or locally, via ingestion, via inhalation, topically, intravenously, or orally.

In another embodiment, the present invention includes a method of determining if a patient has stem cell memory T (T_{scm}) cells that will increase the risk of developing a cardiovascular disease or the severity of an existing cardiovascular disease, comprising: obtaining a biological sample from a patient; and detecting the T_{scm} in the biological sample by contacting the biological sample with an agent that detects T cells expressing CD45RA+CCR7+CD27+CD28+CD95+, or CD45RA+CCR7+CD45RO−CD95+, and determining whether T_{scm} are increased in the biological sample when compared to a baseline level of T_{scm} in a healthy patient, wherein an increase in T_{scm} in the biological sample compared to a healthy patient indicates the patient has cardiovascular disease, is at risk of developing a cardiovascular disease, or has advanced cardiovascular disease. In one aspect, the increase in T_{scm} when compared to the baseline level of T_{scm} in a healthy patient comprises determining the ratio of T_{scm} to naïve T (T_{N}) cells in the patient when compared to the ratio of T_{scm} to Tₙ cells in the healthy patient, wherein the T_{N} are CD8+CD45RA+CCR7+CD27+CD28+ T cells, CD4+CD45RA+CCR7+CD27+CD28+ T cells, or T_{scm} CD8+CD3+CD45RA+CCR7+CD45RO−CD95+ T cells, and wherein at least one of: a higher ratio of T_{scm} to T_{N} in the patient indicates the patient has cardiovascular disease, is at risk of developing a cardiovascular disease, or has advanced cardiovascular disease; a patient with a high risk of myocardial infarction or heart attack (high CVD) has an increase in CD8+ T_{SCM} frequency (% of CD8+ T_{N} cells that are CD8+ T_{SCM} cells) when compared to a patient with a low risk of myocardial infarction or heart attack (low CVD); a patient with a high risk of myocardial infarction or heart attack (high CVD) has an increase of CD8+ T_{SCM} compared to subjects with a low risk of myocardial infarction or heart attack (low CVD); a patient with a high risk of myocardial infarction or heart attack (high CVD) have a decrease in CD8+ T_{N} compared to subjects with a low risk of myocardial infarction or heart attack (low CVD); CD8+ T_{SCM} from subjects with a high risk of myocardial infarction or heart attack (high CVD) have higher expression and release of pro-atherogenic cytokine IFN-γ compared to CD8+ T_{SCM} from subjects with low CVD; or CD8+ T_{SCM} from patients with a high risk of myocardial infarction or heart attack (high CVD) have a higher expression of Ki67 compared to CD8⁺ T_{scm} from subjects with low CVD. In another aspect, a ratio of 1.5:1 T_{scm} to Tₙ cells or greater compared to the ratio of T_{scm} to Tₙ cells in the healthy patient indicates the patient is at risk of developing a cardiovascular disease or having a cardiovascular disease of increased severity. In another aspect, the increase in T_{scm} is determined by detecting T_{scm} in a blood sample. In another aspect, the ratio of T_{scm} and Tₙ are determined by detecting T_{scm} and Tₙ in a blood sample. In another aspect, the T_{N} express one or more of the following markers: CD127, CD11a, CD58, IL-2Rβ, or Bcl-2. In another aspect, the blood sample comprises peripheral blood mononuclear cells (PBMCs). In another aspect, the cardiovascular disease is severe. In another aspect, the T_{scm} are at least one of: CD8+CD45RA+CCR7+CD27+CD28+CD95+ T cells, CD4+CD45RA+CCR7+CD27+CD28+CD95+ T cells, or CD8+CD3+CD45RA+CCR7+CD45RO−CD95+ T cells. In another aspect, the T_{scm} express one or more of the following markers: CXCR3, CD122, LFA-1, c-Myb, Ki67, CD127, CD11a, CD58, IL-2Rβ, or Bcl-2. In another aspect, the increase of T_{scm} in the patient is further determined by measuring an increased expression of IFN-γ in T_{scm} of the patient compared to the expression of IFN-γ in T_{scm} of a healthy patient.

In another embodiment, the present invention includes a method of diagnosing and treating a cardiovascular disease in a patient, the method comprising the steps of: determining whether the patient has an increase in stem cell memory T (T_{scm}) cells by: obtaining or having obtained a biological sample from the patient; performing or having performed an assay on the biological sample to determine the amount of T_{scm} in the patient, wherein the T_{scm} are at least one of: CD8+CD45RA+CCR7+CD27+CD28+CD95+ T cells, CD4+CD45RA+CCR7+CD27+CD28+CD95+ T cells, or CD8+CD3+CD45RA+CCR7+CD45RO−CD95+ T cells; identifying that the patient has an increase in T_{scm} when compared to the amount of T_{scm} in a low-cardiovascular disease (CVDlo) patient; and if the patient has an increase in T_{scm}, then administering therapy for the treatment of the cardiovascular disease to the patient. In one aspect, the diagnosing comprises determining the amount of increase in T_{scm} of the patient compared to the amount of T_{scm} in a CVDlo patient to identify whether the patient has advanced or severe cardiovascular disease. In another aspect, the increase in T_{scm} when compared to the amount of T_{scm} in a CVDlo patient comprises determining the ratio of T_{scm} to naïve T (Tₙ) cells in the patient when compared to the ratio of T_{scm} to Tₙ cells in the CVDlo patient, wherein the Tₙ are CD8+CD45RA+CCR7+CD27+CD28+ T cells, CD4+CD45RA+CCR7+CD27+CD28+ T cells, or CD8+CD3+CD45RA+CCR7+CD45RO−CD95+ T cells, and wherein a higher ratio of T_{scm} to T_{N} in the patient indicates the patient has a greater risk of advanced cardiovascular disease or more advanced cardiovascular disease. In another aspect, a ratio of 1.5:1 T_{scm} to T_{N} cells or greater compared to the ratio of T_{scm} to T_{N} cells in the CVDlo patient indicates the patient has a cardiovascular disease. In another aspect, the amount of T_{scm} is determined by detecting the amount of T_{scm} in a blood sample. In another aspect, the amounts of T_{scm} and Tₙ are determined by detecting the amounts T_{scm} and Tₙ in a blood sample. In another aspect, the T_{N} express one or more of the following markers: CD127, CD11a, CD58, IL-2Rβ, or Bcl-2. In another aspect, the blood sample comprises peripheral blood mononuclear cells (PBMCs). In another aspect, the cardiovascular disease is severe. In another aspect, the T_{scm} express one or more of the following markers: CXCR3, CD122, LFA-1, c-Myb, Ki67, CD127, CD11a, CD58, IL-2Rβ, or Bcl-2. In another aspect, the increase of T_{scm} in the patient is further determined by measuring an increased expression of IFN-γ in T_{scm} of the patient compared to the expression of IFN-γ in T_{scm} of a CVDlo patient, or T_{scm} of a subject with high risk of myocardial infarction or heart attack (high CVD) express higher levels of Ki67 and IFN-γ than T_{scm} of a subject with low risk of myocardial infarction or heart attack (low CVD). In another aspect, the therapy for the treatment of the cardiovascular disease comprises a T_{scm}, T_{N}, or TCD25 modulating therapy. In another aspect, the T_{scm}, T_{N}, or TCD25 modulating therapy is a T_{scm} depleting therapy, a T_{N} increasing therapy, or a T_{CD25} CD8+CD3+CD25⁺ T cell therapy. In another aspect, the therapy for the treatment of the cardiovascular disease is administered systemically, regionally or locally, via ingestion, via inhalation, topically, intravenously, or orally.

In another embodiment, the present invention includes a method for treating a patient suffering from advanced or severe cardiovascular disease, the method comprising the steps of: determining whether the patient has an increase in stem cell memory T (T_{scm}) cells, when compared to a baseline level of T_{scm} present in a low-cardiovascular disease (CVDlo) patient by: obtaining or having obtained a biological sample from the patient; and performing or having performed an assay on the biological sample to determine if the patient has an increase in T_{scm} when compared to a baseline level of T_{scm} present in CVDlo patient; and if the patient has an increase in T_{scm} when compared to a baseline level of T_{scm} present in a CVDlo patient, then administering therapy for the treatment of the cardiovascular disease to the patient. In one aspect, the increase in T_{scm} when compared to the baseline level of T_{scm} in a healthy patient comprises determining the ratio of T_{scm} to naïve T (T_{N}) cells in the patient when compared to the ratio of T_{scm} to Tₙ cells in the healthy patient, wherein the T_{N} are CD8+CD45RA+CCR7+CD27+CD28+ T cells, CD4+CD45RA+CCR7+CD27+CD28+ T cells, or T_{scm} CD8+CD3+CD45RA+CCR7+CD45RO−CD95+ T cells, and wherein at least one of: a higher ratio of T_{scm} to T_{N} in the patient indicates the patient has cardiovascular disease, is at risk of developing a cardiovascular disease, or has advanced cardiovascular disease; a patient with a high risk of myocardial infarction or heart attack (high CVD) has an increase in CD8⁺ T_{SCM} frequency (% of CD8⁺ T_{N} cells that are CD8⁺ T_{SCM} cells) when compared to a patient with a low risk of myocardial infarction or heart attack (low CVD); a patient with a high risk of myocardial infarction or heart attack (high CVD) has an increase of CD8⁺ T_{SCM} compared to subjects with a low risk of myocardial infarction or heart attack (low CVD); a patient with a high risk of myocardial infarction or heart attack (high CVD) have a decrease in CD8⁺ T_{N} compared to subjects with a low risk of myocardial infarction or heart attack (low CVD); CD8⁺ T_{SCM} from subjects with a high risk of myocardial infarction or heart attack (high CVD) have higher expression and release of pro-atherogenic cytokine IFN-γ compared to CD8+ T_{scm} from subjects with low CVD; or CD8+ T_{scm} from patients with a high risk of myocardial infarction or heart attack (high CVD) have a higher expression of Ki67 compared to CD8+ T_{scm} from subjects with low CVD. In another aspect, a ratio of 1.5:1 T_{scm} to T_{N} cells or greater compared to the ratio of T_{scm} to T_{N} cells in the CVDlo patient indicates the patient should be treated for the cardiovascular disease. In another aspect, the increase in T_{scm} is determined by detecting T_{scm} in a blood sample. In another aspect, the ratio of T_{scm} and T_{N} is determined by detecting T_{scm} and T_{N} in a blood sample. In another aspect, the T_{N} express one or more of the following markers: CD127, CD11a, CD58, IL-2Rβ, or Bcl-2. In another aspect, the blood sample comprises peripheral blood mononuclear cells (PBMCs). In another aspect, the cardiovascular disease is severe. In another aspect, the T_{scm} are CD8+CD45RA+CCR7+CD27+CD28+CD95+ T cells, CD4+CD45RA+CCR7+CD27+CD28+CD95+ T cells, or CD8+CD3+CD45RA+CCR7+CD45RO−CD95+ T cells. In another aspect, the T_{scm} express one or more of the following markers: CXCR3, CD122, LFA-1, c-Myb, Ki67, CD127, CD11a, CD58, IL-2Rβ, or Bcl-2. In another aspect, the increase of T_{scm} in the patient is further determined by measuring an increased expression of IFN-gamma in T_{scm} of the patient compared to the expression of IFN-gamma in T_{scm} of a CVDlo patient, or T_{scm} of a subject with high risk of myocardial infarction or heart attack (high CVD) express higher levels of Ki67 and IFN-γ than T_{scm} of a subject with low risk of myocardial infarction or heart attack (low CVD). In another aspect, the therapy for the treatment of cardiovascular disease comprises a T_{scm}, T_{N}, or T_{CD25} modulating therapy. In another aspect, the T_{scm}, T_{N}, or T_{CD25} modulating therapy is a T_{scm} depleting therapy, a T_{N} increasing therapy, or a T_{CD25} CD8+CD3+CD25+ T cell therapy. In another aspect, the therapy for the treatment of the cardiovascular disease is administered systemically, regionally or locally, via ingestion, via inhalation, topically, intravenously, or orally.

In another embodiment, the present invention includes a method of determining if a patient has stem cell memory T (T_{scm}) cells that will increase the risk of developing advanced cardiovascular disease or indicates an existing advanced or severe cardiovascular disease, comprising: obtaining a biological sample from a patient; and detecting the T_{scm} in the biological sample by contacting the biological sample with an agent that detects T cells expressing CD45RA+CCR7+CD27+CD28+CD95+ or CD45RA+CCR7+CD45RO−CD95+, and determining whether T_{scm} are increased in the biological sample when compared to a baseline level of T_{scm} in a low-cardiovascular disease (CVDlo) patient, wherein an increase in T_{scm} in the biological sample compared to a CVDlo patient indicates the patient has advanced or severe cardiovascular disease, or is at risk of developing advanced or severe cardiovascular disease. In one aspect, the increase in T_{scm} when compared to the baseline level of T_{scm} in a healthy patient comprises determining the ratio of T_{scm} to naïve T (T_{N}) cells in the patient when compared to the ratio of T_{scm} to Tₙ cells in the healthy patient, wherein the T_{N} are CD8+CD45RA+CCR7+CD27+CD28+ T cells, CD4+CD45RA+CCR7+CD27+CD28+ T cells, or T_{scm} CD8+CD3+CD45RA+CCR7+CD45RO−CD95+ T cells, and wherein at least one of: a higher ratio of T_{scm} to T_{N} in the patient indicates the patient has cardiovascular disease, is at risk of developing a cardiovascular disease, or has advanced cardiovascular disease; a patient with a high risk of myocardial infarction or heart attack (high CVD) has an increase in CD8+ T_{SCM} frequency (% of CD8+ T_{N} cells that are CD8+ T_{SCM} cells) when compared to a patient with a low risk of myocardial infarction or heart attack (low CVD); a patient with a high risk of myocardial infarction or heart attack (high CVD) has an increase of CD8+ T_{SCM} compared to subjects with a low risk of myocardial infarction or heart attack (low CVD); a patient with a high risk of myocardial infarction or heart attack (high CVD) have a decrease in CD8+ T_{N} compared to subjects with a low risk of myocardial infarction or heart attack (low CVD); CD8+ T_{SCM} from subjects with a high risk of myocardial infarction or heart attack (high CVD) have higher expression and release of pro-atherogenic cytokine IFN-γ compared to CD8+ T_{SCM} from subjects with low CVD; or CD8+ T_{SCM} from patients with a high risk of myocardial infarction or heart attack (high CVD) have a higher expression of Ki67 compared to CD8+ T_{scm} from subjects with low CVD. In another aspect, a ratio of 1.5:1 T_{scm} to T_{N} cells or greater compared to the ratio of T_{scm} to T_{N} cells in the CVDlo patient indicates the patient is at risk of developing a cardiovascular disease or having a cardiovascular disease of increased severity. In another aspect, the increase in T_{scm} is determined by detecting T_{scm} in a blood sample. In another aspect, the ratio of T_{scm} and T_{N} is determined by detecting T_{scm} and T_{N} in a blood sample. In another aspect, the T_{N} express one or more of the following markers: CD127, CD11a, CD58, IL-2Rβ, or Bcl-2. In another aspect, the blood sample comprises peripheral blood mononuclear cells (PBMCs). In another aspect, the cardiovascular disease is severe. In another aspect, the T_{scm} are CD8+CD45RA+CCR7+CD27+CD28+CD95+ T cells, CD4+CD45RA+CCR7+CD27+CD28+CD95+ T cells, or CD8+CD3+CD45RA+CCR7+CD45RO−CD95+ T cells. In another aspect, the T_{scm} express one or more of the following markers: CXCR3, CD122, LFA-1, c-Myb, Ki67, CD127, CD11a, CD58, IL-2Rβ, or Bcl-2. In another aspect, the increase of T_{scm} in the patient is further determined by measuring an increased expression of IFN-γ in T_{scm} of the patient compared to the expression of IFN-gamma in T_{scm} of a CVDlo patient, or T_{scm} of a subject with high risk of myocardial infarction or heart attack (high CVD) express higher levels of Ki67 and IFN-γ than T_{scm} of a subject with low risk of myocardial infarction or heart attack (low CVD). In another aspect, the agent is an antibody, a small molecule, a protein, a peptide, a ligand mimetic or a nucleic acid.

In another embodiment, the present invention includes a method of diagnosing and treating a cardiovascular disease in a patient, the method comprising the steps of: determining whether the patient has a higher ratio of stem cell memory T (T_{scm}) cells to naïve T (T_{N}) cells by: obtaining or having obtained a biological sample from the patient; performing or having performed an assay on the biological sample to determine the amount of T_{scm} in the patient, wherein the T_{scm} are CD8+CD45RA+CCR7+CD27+CD28+CD95+ T cells, CD4+CD45RA+CCR7+CD27+CD28+CD95+ T cells, or CD8+CD3+CD45RA+CCR7+CD45RO−CD95+ T cells, and the amount of T_{N} in the patient, wherein the T_{N} are CD8+CD3+CD45RA+CCR7+CD45RO−CD95−; determining the ratio of T_{scm} to T_{N} cells in the patient when compared to the ratio of T_{scm} to T_{N} cells in a healthy patient or a low-cardiovascular disease (CVDlo) patient; and if the patient has a higher ratio of T_{scm} to T_{N} than the ratio of T_{scm} to T_{N} in the healthy patient or CVDlo patient, administering therapy for the treatment of the cardiovascular disease to the patient. In one aspect, the diagnosing comprises determining the ratio of T_{scm} to T_{N} in the patient compared to the ratio of T_{scm} to T_{N} in the healthy patient or CVDlo patient to identify whether the patient has cardiovascular disease, is at risk of developing cardiovascular disease, or has advanced or severe cardiovascular disease. In another aspect, a ratio of 1.5:1 T_{scm} to T_{N} cells or greater in the patient compared to the ratio of T_{scm} to T_{N} in the healthy patient or CVDlo patient indicates the patient has a cardiovascular disease. In another aspect, the amounts of T_{scm} and T_{N} are determined by detecting the amounts T_{scm} and T_{N} in a blood sample. In another aspect, the blood sample comprises peripheral blood mononuclear cells (PBMCs). In another aspect, the T_{N} express one or more of the following markers: CD127, CD11a, CD58, IL-2Rβ, or Bcl-2. In another aspect, the cardiovascular disease is severe. In another aspect, the T_{scm} express one or more of the following markers: CXCR3, CD122, LFA-1, c-Myb, Ki67, CD127, CD11a, CD58, IL-2Rβ, or Bcl-2. In another aspect, the amount of T_{scm} in the patient is further determined by measuring expression of IFN-γ in T_{scm} of the patient compared to the expression of IFN-γ in T_{scm} of a healthy patient or CVDlo patient, or T_{scm} of a subject with high risk of myocardial infarction or heart attack (high CVD) express higher levels of Ki67 and IFN-γ than T_{scm} of a subject with low risk of myocardial infarction or heart attack (low CVD). In another aspect, the therapy for the treatment of the cardiovascular disease comprises a T_{scm}, T_{N}, or TCD25 modulating therapy. In another aspect, the T_{scm} or T_{N} modulating therapy is a T_{scm} depleting therapy, a T_{N} increasing therapy, or a T_{CD25} CD8+CD3+CD25+ T cell. In another aspect, the therapy for the treatment of the cardiovascular disease is administered systemically, regionally or locally, via ingestion, via inhalation, topically, intravenously, or orally.

In another embodiment, the present invention includes a method for treating a patient suffering from cardiovascular disease, the method comprising the steps of: determining whether the patient has a higher ratio of stem cell memory T (T_{scm}) cells to naïve T (T_{N}) cells, compared to the ratio of T_{scm} to T_{N} cells in a healthy patient or a low-cardiovascular disease (CVDlo) patient, by: obtaining or having obtained a biological sample from the patient; and performing or having performed an assay on the biological sample to determine the ratio of T_{scm} to Tₙ cells in the patient when compared to the ratio of T_{scm} to T_{N} cells in a healthy patient or a low-cardiovascular disease (CVDlo) patient; and if the patient has a higher ratio of T_{scm} to T_{N} than the ratio of T_{scm} to T_{N} in the healthy patient or CVDlo patient, administering therapy for the treatment of the cardiovascular disease to the patient. In one aspect, the T_{scm} are CD8+CD45RA+CCR7+CD27+CD28+CD95+ T cells, CD4+CD45RA+CCR7+CD27+CD28+CD95+ T cells, or CD8+CD3+CD45RA+CCR7+CD45RO−CD95+ T cells, and wherein the T_{N} are CD8+CD3+CD45RA+CCR7+CD45RO−CD95−. In another aspect, a ratio of 1.5:1 T_{scm} to T_{N} cells or greater compared to the ratio of T_{scm} to T_{N} cells in a healthy patient or a low-cardiovascular disease (CVDlo) patient indicates the patient should be treated for the cardiovascular disease. In another aspect, the ratio of T_{scm} and T_{N} are determined by detecting T_{scm} and T_{N} in a blood sample. In another aspect, the blood sample comprises peripheral blood mononuclear cells (PBMCs). In another aspect, the T_{N} express one or more of the following markers: CD127, CD11a, CD58, IL-2Rβ, or Bcl-2. In another aspect, the cardiovascular disease is severe. In another aspect, the T_{scm} express one or more of the following markers: CXCR3, CD122, LFA-1, c-Myb, Ki67, CD127, CD11a, CD58, IL-2Rβ, or Bcl-2. In another aspect, the level of T_{scm} in the patient is further determined by measuring an increased expression of IFN-gamma in T_{scm} of the patient compared to the expression of IFN-gamma in T_{scm} of a healthy patient or a CVDlo patient, or T_{scm} of a subject with high risk of myocardial infarction or heart attack (high CVD) express higher levels of Ki67 and IFN-γ than T_{scm} of a subject with low risk of myocardial infarction or heart attack (low CVD). In another aspect, the therapy for the treatment of cardiovascular disease comprises a T_{scm}, T_{N}, or TCD25 modulating therapy. In another aspect, T_{scm} or T_{N} modulating therapy is a T_{scm} depleting therapy, a Tₙ increasing therapy, or a T_{CD25} CD8+CD3+CD25+ T cell therapy. In another aspect, therapy for the treatment of the cardiovascular disease is administered systemically, regionally or locally, via ingestion, via inhalation, topically, intravenously, or orally.

In another embodiment, the present invention includes a method of determining if a patient has stem cell memory T (T_{scm}) cells that will increase the risk of developing a cardiovascular disease or the severity of an existing cardiovascular disease, comprising: obtaining a biological sample from a patient; and detecting the ratio of T_{scm} to naïve T (T_{N}) cells in the biological sample by contacting the biological sample with an agent that detects T cells expressing CD45RA+CCR7+CD27+CD28+CD95+, CD45RA+CCR7+CD27+CD28+CD95−/CD45RA+CCR7+CD27+CD28+CD95lo, or CD45RA+CCR7+CD45RO−CD95+ and determining the ratio of T_{scm} to Tₙ in the biological sample when compared to the ratio of T_{scm} to T_{N} cells in a healthy patient or a low-cardiovascular disease (CVDlo) patient, wherein a higher ratio of T_{scm} to T_{N} in the biological sample compared to the ratio of T_{scm} to T_{N} in the healthy patient or CVDlo patient indicates the patient has cardiovascular disease, is at risk of developing a cardiovascular disease, or has advanced cardiovascular disease. In one aspect, the T_{scm} are CD8+CD45RA+CCR7+CD27+CD28+CD95+ T cells, CD4+CD45RA+CCR7+CD27+CD28+CD95+ T cells, or CD8+CD3+CD45RA+CCR7+CD45RO−CD95+ T cells, and wherein the T_{N} are CD8+CD3+CD45RA+CCR7+CD45RO−CD95− T cells. In another aspect, a ratio of 1.5:1 T_{scm} to T_{N} cells or greater compared to the ratio of T_{scm} to T_{N} cells in a healthy patient or a low-cardiovascular disease (CVDlo) patient indicates the patient is at risk of developing a cardiovascular disease or having a cardiovascular disease of increased severity. In another aspect, the ratio of T_{scm} and T_{N} are determined by detecting T_{scm} and T_{N} in a blood sample. In another aspect, the blood sample comprises peripheral blood mononuclear cells (PBMCs). In another aspect, the T_{N} express one or more of the following markers: CD127, CD11a, CD58, IL-2Rβ, or Bcl-2. In another aspect, the cardiovascular disease is severe. In another aspect, the T_{scm} express one or more of the following markers: CXCR3, CD122, LFA-1, c-Myb, Ki67, CD127, CD11a, CD58, IL-2Rβ, or Bcl-2. In another aspect, the level of T_{scm} in the patient is further determined by measuring an increased expression of IFN-γ in T_{scm} of the patient compared to the expression of IFN-γ in T_{scm} of a healthy patient or a CVDlo patient, or T_{scm} of a subject with high risk of myocardial infarction or heart attack (high CVD) express higher levels of Ki67 and IFN-γ than T_{scm} of a subject with low risk of myocardial infarction or heart attack (low CVD). In another aspect, the agent is an antibody, a small molecule, a protein, a peptide, a ligand mimetic or a nucleic acid.

In another embodiment, the present invention includes a method of treating a patient with an increased risk of developing a cardiovascular disease or has existing cardiovascular disease, comprising: determining a ratio of T_{scm} to T_{N} from a biological sample obtained from the patient, wherein a higher ratio of T_{scm} to T_{N} in the biological sample compared to the ratio of T_{scm} to T_{N} in a healthy patient or CVDlo patient indicates the patient is in need of treatment to prevent or treat the cardiovascular disease; and treating the patient with a T_{scm}, T_{N}, or T_{CD25} modulating therapy. In one aspect, the T_{scm} or T_{N} modulating therapy is a T_{scm} depleting therapy, a T_{N} increasing therapy, or a T_{CD25} CD8+CD3+CD25+ T cell therapy. In another aspect, the therapy for the treatment of the cardiovascular disease is administered systemically, regionally or locally, via ingestion, via inhalation, topically, intravenously, or orally. In another aspect, the therapy for the treatment of the cardiovascular disease is adoptive T cell therapy, chimeric antigen receptor T cell (CAR-T) therapy, antibody dependent cell cytotoxicity therapy, antibody drug conjugate therapy, T-cell receptor (TCR) modified T-cell therapy, chimeric antigen receptor (CAR)-modified natural killer cell therapy, administration of monoclonal antibodies, administration of cytokines, T cell suppressing therapy, or any combination thereof.

### BRIEF DESCRIPTION OF FIGURES

For a more complete understanding of the features and advantages of the present invention, reference is now made to the detailed description of the invention along with the accompanying figures and in which:

FIGS. 1A and 1B. Experimental Design for examining T cell heterogeneity within low and high CVD individuals. PBMCs from low (Gensini score<10) and high (Gensini score>30) CVD individuals were stained with a validated, T cell-focused panel of rare earth metal-conjugated antibodies (FIG. 1A, FIG. 1B). CyTOF mass cytometry was performed, and high dimensional clustering analyses were utilized.

FIGS. 2A to 2D. FlowSOM clustering and differential abundance test identifies a naïve CD8⁺ T cell subset as severely attenuated in low versus high CVD individuals. FlowSOM and consensus clustering was performed on CD3⁺ T cells of low (n=14) and high (n=14) CVD individuals, resulting in 15 T cell subsets. CD3⁺ T cells were projected onto a 2-dimensional map using UMAP, a non-linear reduction method (FIG. 2A). Heatmap of median expression of 22 markers after scaling normalization across 15 subsets (FIG. 2B). Volcano plot of −log 10(pvalue) and log 2 fold-change from differential abundance test (diffcyt, see Method section) for 15 clusters (FIG. 2C) identified cluster 3 as the most differentially expressed between low and high CVD individuals (FIG. 2D).

FIGS. 3A to 3F. High CVD individuals display attenuated naïve CD8⁺ T cell frequencies in the periphery, and increased CD95. Representative contour plots of CD45A by CCR7, gated on CD8⁺ T cells (FIG. 3A), pooled frequencies of naive CD8⁺ T cells out of CD8⁺ T cells (FIG. 3B) and CD45⁺ leukocytes (FIG. 3C). FIGS. 3D-3F show representative contour plots of CD8 by CD95, gated on naive CD8⁺ T cells. Pooled frequencies of CD8⁺CD95⁺ T cells, gated on naive T cells and CD95 gMFI. Results are expressed as mean±s.e.m from 14 low and 14 high CVD individuals. Statistical significance was calculated using an unpaired t-test.

FIGS. 4A to 4E. High CVD individuals display increased T_{SCM} frequencies. Representative contour plots of CCR7 by CD95, gated on CD45RA+CD27+CD127+CD45ROlo T cells (FIG. 4A), pooled frequencies of CD8+ T_{SCM} cells out of CD8+ T_{N} cells (FIG. 4B) and CD8+ T cells (FIG. 4C) for age-matched healthy controls (n=10), low CVD (n=14), and high CVD (n=14) individuals. Pearson correlations of Gensini score by CD8+ T_{SCM} (FIG. 4D) and CD8+ T_{N} frequencies (FIG. 4E). Results are expressed mean±s.e.m for two independent experiments. Statistical significance was calculated using unpaired t-test.

FIGS. 5A to 5F. T_{SCM} cells display enhanced proliferative capacity, and high CVD T_{SCM} cells secrete enhanced IFN-γ. Representative contour plots of CTV by SSC, gating on CD8+ T cells for sorted subsets (FIG. 5A) and pooled frequencies of CTV-diluted cells for T_{N} and T_{SCM} cells from low (n=2) and high (n=2) CVD individuals (FIG. 5B). Pooled frequencies of CD8+Ki67+ T cells, gating on CD8+ T cells for sorted T_{N} and T_{SCM} subsets from low (n=5) and high (n=5) CVD individuals (FIG. 5C). Representative contour plots of IFN-γ by CD8, gating on CD8+ T_{SCM} cells (FIG. 5D). Pooled frequencies of CD8⁺IFN-γ+ T cells (FIG. 5E) and IFN-γ gMFI (FIG. 5F), gated on CD8+ T_{SCM} (CD8+CD45RA+CCR7+CD95+) cells from low (n=8) and high (n=8) CVD individuals. Results are expressed as mean±s.e.m for one independent experiment (FIG. 5B) and 2 independent experiments (FIGS. 5C, 5E, 5F). Statistical significance was calculated using an unpaired t-test.

FIGS. 6A to 6D. T_{SCM} cells are enriched in ApoE−/− mice. Representative contour plots of Bcl-2 by Sca-1, gating on CD8+ T_{N} cells (CD44lowCD62Lhigh) within blood (FIG. 6A) and para-aortic lymph nodes (pa-LNs) (FIG. 6C). Pooled T_{SCM} cell frequencies, expressed as a percentage of total CD8+ T cells within blood (FIG. 6B) and pa-LN (FIG. 6D) within B6 (n=5) and ApoE−/− (n=5) mice. Results are expressed as mean±s.e.m for one of two independent experiments. Statistical significance was calculated using an unpaired t-test.

FIGS. 7A to 7H. T_{SCM} cells are increased in early compared to advanced stages of atherosclerosis. Representative contour plots of Bcl-2 by Sca-1, gating on CD8+ T_{N} cells (CD44lowCD62Lhigh) within blood (FIG. 7A), pa-LNs (FIG. 7C), aorta (FIG. 7E), and spleen (FIG. 7G) and pooled T_{SCM} cell frequencies out of CD8+ T_{N} within blood (FIG. 7B), pa-LN (FIG. 7D) aorta (FIG. 7F), and spleen (FIG. 7H) at early (n=4) and advanced (n=4) stages of atherosclerosis. Results are expressed as mean±s.e.m for one of two independent experiments. Statistical significance was calculated using an unpaired t-test and statistically significant differences were at *p<0.05, **P<0.01.

FIGS. 8A to 8C. T_{SCM} adoptive transfer elicits increased atherosclerosis. Oil Red O staining of aortic roots from T_{N}, T_{SCM}, and T_{EM} Rag.Ldlr recipients at 12 weeks post high cholesterol diet (FIG. 8A). Pooled quantitation of Oil Red O-Positive cells for T_{N}, T_{SCM}, and T_{EM} transfer recipients (FIG. 8B). Weight (in grams) of adoptive transfer recipients during twelve weeks on high cholesterol diet post adoptive transfer (FIG. 8C). Results are expressed as mean±s.e.m for one independent experiment.

FIGS. 9A and 9B. CD8 T_{SCM} cells display enhanced BrdU positivity within the aorta-draining para-aortic lymph nodes in WD− compared to chow diet-fed ApoE−/− mice. Frequencies of BrdU+ cells (Bromodeoxyuridine/5-bromo-2′-deoxyuridine), gated on CD8 T_{SCM} cells (CD8+CD44lowCD62Lhigh) within the paLN (FIG. 9A), inguinal LN (ing LN) (FIG. 9B), within chow and WD-fed ApoE−/− mice for 4 weeks. Mice were injected with BrdU (100 μg/mL) for 24 hrs prior to detection via flow cytometry.

FIG. 10. CD8 T_{N} cells undergo proliferation and differentiation when exposed to oxLDL. Sorted CD8 T_{N} cells were incubated with PE-labelled oxLDL, HDL, or LDL in the presence of TL-7 and IL-15 (25 ng/mL). Representative contour plots of CD95 by Ki67, gated on oxLDL-, HDL-, or LDL-positive cells (A) and pooled percentages of CD95+Ki67+ T cells (B).

FIGS. 11A and 11B. Workflow for high-dimensional mass cytometry of CD8 T cells in human CVD. PBMCs were stained with a CyTOF T cell panel, and mass cytometry was performed. Analysis via UMAP showed 7 distinct CD8 T cell clusters in blood. In the UMAP each dot is a cell and each colored cluster is a phenotypically different cluster of CD8 T cells identified using this method (FIG. 11A). CITRUS identified key cell profiles (shown in red and light green dots) distinguishing low and high CVD-risk individuals. (FIG. 11B).

FIGS. 12A and 12B. High-risk CVD subjects have increased T_{SCM} cell frequencies in blood. Representative contour plots of CCR7 by CD95, gated on CD454RA+CD27+CD127+CD45ROlow cells (FIG. 12A), frequencies of CD8 T_{SCM} cells, normalized to lymphocyte count (k/μL) (FIG. 12B).

FIG. 13. CD8 T_{SCM} cells correlate with CVD severity. Pearson's correlation analysis of T_{SCM} frequencies with clinical Gensini scores reveals a positive correlation of CD8 T_{SCM} with CVD severity. Each dot represents one subject.

FIG. 14. CD8 T subset changes in low vs high-risk CVD subjects. CyTOF analysis of the CAVA cohort identified a senescent CD8 T cell subset (CD57⁺; left) that was increased in high CVD subjects and a regulatory subset (CD25⁺; right) that was slightly decreased in high CVD subjects. P value obtained by unpaired Student's t-test.

FIG. 15. Subsets of human CD8 T cell subsets defined in healthy subjects by high-dimensional Ab-Seq to demonstrate technical feasibility. Analysis of Abseq data from 6,629 PBMCs from healthy subjects revealed 2 unique naive subsets of CD8 T cells among 14 clusters identified on the basis of both protein (left panel) and gene expression (right panel).

FIG. 16. T_{SCM} adoptive transfer increases atherosclerosis in vivo. Experimental schematic for CD8 T_{N}, CD8 T_{SCM}, bulk CD8 T cells and control CD4 Treg into RAG.Ldlr recipients (A). Representative Oil Red O staining of aortic arches from CD8 T_{N}, T_{SCM}, CD8 T cells and Treg recipient mice at 12 weeks post high cholesterol diet feeding (B). Pooled quantitation of Oil Red-O positive cells for T_{N} (n=8), T_{SCM}(n=5), bulk CD8 (n=5) and Treg (n=2) transfer recipients (C). Results are expressed as mean+/−sem for 3 independent experiments. Statistical significance was calculated by unpaired Student's t-test (*p<0.05).

FIG. 17. oxLDL incubation of CD8 T_{N} elicits an increase in T_{SCM}-like T cells. Sorted Apoe−/− CD8 T_{N} cells (CD44lowCD62LhighSca−/−) were incubated with 10 μg/mL oxLDL for 72 hours. oxLDL induces CD8 TSCM differentiation.

### DETAILED DESCRIPTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity but include the general class of which a specific example may be used for illustration. The terminology herein is used to describe specific embodiments of the invention, but their usage does not delimit the invention, except as outlined in the claims.

As used herein, stem cell memory (T_{SCM}, T_{SCM} or T_{scm}) T cells refers to T cells that express CD45RA+CCR7+CD27+CD28+CD95+, which are naïve markers [CD45RA+CCR7+CD27+CD28+] with memory antigen CD95+. For example, the T_{scm} are CD8+CD45RA+CCR7+CD27+CD28+CD95+, CD4+CD45RA+CCR7+CD27+CD28+CD95+, or CD8+CD3+CD45RA+CCR7+CD45RO−CD95+ T cells. Other T_{scm}-specific markers include at least one of: CD127 CD11a, CD58, IL-2Rβ, Bcl-2, CXCR3, CD122, LFA-1, or c-Myb. T_{scm} of a subject with high risk of myocardial infarction or heart attack (high CVD) express higher levels of Ki67 and IFN-γ than T_{scm} of a subject with low risk of myocardial infarction or heart attack (low CVD).

As used herein, naïve T cells (T_{N}, T_{N} or Tₙ) refers to T cells that express CD45RA+CCR7+CD27+CD28+, but can also be: CD8+CD3+CD45RA+CCR7+CD27+CD28+, CD4+CD3+CD45RA+CCR7+CD27+CD28+, or CD8+CD3+CD45RA⁺CCR7+CD45RO⁻CD95⁻. T_{N}can also have other naïve markers: CD127 CD11a, CD58, IL-2Rβ, Bcl-2 (shared between T_{scm} and T_{N}).

As used herein, the term CD8 T_{CD25} T cells refers to a population of CD8+ T cells identified to be atheroprotective and/or are a biomarker of CVD risk. For example, the CD8 T_{CD25} are CD8+CD3+CD25+ T cells. The CD8 T_{CD25} T cells can be used to provide an atheroprotective therapy. The loss of CD8 T_{CD25} T cells is a biomarker for increase CVD risk and/or the presence of CVD.

Methods of measuring T_{SCM} and T_{N} to assess/detect/determine/predict CVD disease severity and/or progression (T cells are isolated from PBMCs of a subject), include: (1) patients with high risk of myocardial infarction or heart attack (high CVD) have a [1.5-fold] increase in CD8+ T_{SCM} frequency (% of CD8+ T_{N} cells that are CD8+ T_{SCM} cells) compared to subjects with a low risk of myocardial infarction or heart attack (low CVD); (2) patients with high risk of myocardial infarction or heart attack (high CVD) have an [2-3 fold] increase of CD8+ T_{SCM} compared to subjects with a low risk of myocardial infarction or heart attack (low CVD); (3) patients with high risk of myocardial infarction or heart attack (high CVD) have a [3-fold] decrease in CD8+ T_{N} compared to patients with a low risk of myocardial infarction or heart attack (low CVD); (4) CD8+ T_{SCM} from patients with a high risk of MI or heart attack (high CVD) have [2-fold] higher expression and release of pro-atherogenic cytokine IFN-γ compared to CD8+ T_{SCM} from patients with low CVD; or (5) CD8+ T_{SCM} from patients with a high risk of MI or heart attack (high CVD) have [2-fold] higher expression of Ki67 compared to CD8+ T_{SCM} from patients with low CVD.

Methods of measuring CVD: (1) Gensini scores−angiography-derived scores that measure atheroprogression. Gensini scores>20 are considered high-risk CVD subjects; and/or (2) coronary artery calcium (CAC) Agatson scores−CAC scores are positively correlated with increased CVD and MI risk; low (CAC scores=0) and high (CAC scores>300).

The present inventors have discovered that CD8+ T_{SCM} cells positively correlated with CVD risk, while CD8+ T_{N} cells were inversely correlated. CD8+ T_{SCM} from severe CVD individuals showed 2-fold higher release of pro-atherogenic cytokine IFN-γ and increased IFN-γ gMFI compared to those with low CVD. Enhanced T_{SCM} frequencies were observed not only in humans, but also in atherosclerotic mice. Apolipoprotein E-deficient (ApoE−/−) mice displayed 7- and 2-fold increased CD8 T_{SCM} frequencies, respectively, within peripheral blood and aorta-draining para-aortic lymph nodes compared to C57BL/6 mice. Importantly, CD8 T_{SCM} cells were 3.2-fold increased in ApoE−/− mouse aortas in early compared to advanced stages of atherosclerosis, and transfer of TSCM cells into immune-deficient Rag.Ldlr recipients elicited increased atherosclerosis disease severity, indicating that this novel population is pro-atherogenic. Thus, CD95 expression by naive T cells, either CD8+ or CD4+, constitute a predictive diagnostic indicator of CVD, potentially improving diagnosis of at-risk individuals.

### Example 1. Individuals at High Risk for CVD Displayed Severely Attenuated Naive (TN) CD8+ T Cell Frequencies in the Peripheral Blood

The inventors demonstrated that individuals at high risk for CVD displayed severely attenuated naive (T_{N}) CD8+ T cell frequencies in the peripheral blood. Examination of the CD8+ T_{N} cell compartment revealed markedly increased expression of the memory antigen CD95 on CD8+ T_{N} cells in high compared to low CVD individuals. Stem cell memory T (T_{SCM}) cells, comprising approximately 2-3% of the circulating T cell pool in healthy individuals have been identified in mice, non-human primates, and humans and are denoted by expression of the naive markers (CD45RA, CCR7, CD27, CD28, and CD127)¹⁹. The inventors show herein that these CD8+ T_{SCM} cells are pro-atherogenic and a biomarker of CVD risk.

The inventors found that CD8+ T_{SCM} cells positively correlated with clinical Gensini score and CVD severity, while CD8+ T_{N} cells were inversely correlated. CD8+ T_{SCM} cells from high CVD individuals displayed increased synthesis of the pro-atherogenic cytokine IFN-γ. Moreover, atherosclerotic apolipoprotein-E-deficient (ApoE−/−) displayed increased T_{SCM} frequencies within peripheral blood and aorta-draining para-aortic lymph nodes compared to C57BL/6, and CD8+T_{SCM} cells were enhanced in ApoE^{-/-} aortas in early compared to advanced stages of atherosclerosis. Importantly CD8+ TSCM cell adoptive transfer elicited increased atherosclerosis disease severity in Rag.Ldlr recipients compared to effector memory CD8+ T cells, indicating that this novel population is pro-atherogenic. In this study, the inventors observed reduced frequencies of naive CD8⁺ T (T_{N}) cells in the blood of individuals with high compared to low CVD. This reduction in CD8⁺ T_{N} cells was discovered utilizing non-biased high dimensional mass cytometry. Some clinical studies have reported on a loss of CD8⁺ T_{N} cells in Individuals with acute coronary syndrome (ACS) 35. Moreover, CD8⁺ T_{N} cells were enriched in patients without severe coronary disease, whereas frequencies were attenuated in individuals with advanced atherosclerosis³⁶. This reduction in CD8⁺ T_{N} cells could be explained by a number of diverse mechanisms, such as decreased survival and/or an increased propensity to differentiate into memory subsets. In support of the latter, in the present disclosure the inventors observed elevated levels of the memory antigen CD95 on CD8⁺ T_{N} cells and increased CD8⁺CD95⁺ frequencies in individuals with high versus low CVD individuals.

CD95 or Fas (Apo-1), a protein expressed on the cell surface, initiates cellular apoptosis by inhibiting a protease cascade. Resting T cells express low levels of CD95, but following T cell activation, expression is markedly enhanced. While one hallmark of apoptotic cells is CD95 expression, Fas also elicits proliferation of cancer stem cells via TCF-β catenin signalling, which is essential for the formation of memory subsets³⁷.

The inventors found increased frequencies of CD8⁺ T_{SCM} cells, CD8⁺ T_{N} cells expressing CD95. CD8⁺ T_{SCM} cells were enriched in the periphery of subjects with high CVD compared to subjects with low CVD. Specifically, CD8⁺ T_{SCM} frequencies were elevated as a frequency of CD8⁺ T_{N} cells but not out of total CD8⁺ T cells. This is perhaps due to the attenuated CD8⁺ T_{N} cell frequencies within high CVD individuals. Interestingly, CD8⁺ T_{SCM} within high and low CVD individuals were significantly enhanced as a function of the total CD8+ T cell compartment compared to age-matched healthy controls. This also mirrored the findings within the ApoE^{-/-} mouse strain, a mouse model of atherosclerosis, in which CD8⁺ T_{SCM} frequencies were significantly increased compared to C57BL/6 control mice.

The proliferative capacity of CD8⁺ T_{SCM} cells is well documented^{19,26,31}. The inventors found that the CD8⁺ T_{SCM} cells displayed a slight increase in CTV dilution compared to CD8⁺ T_{N} cells. Interestingly, T_{SCM} cells were inherently more proliferative than T_{N} cells. CD8⁺ T_{N} cells isolated from individuals with CVD displayed features of functional exhaustion, such as increased PD-1 and impaired IL-2 production compared to healthy controls 35. Without being limited to a particular theory, this functional exhaustion could be the reason that no increase in proliferative capacity was observed when comparing stimulated T_{N} to T_{SCM} cells.

Adoptive transfer of CD8⁺ T_{SCM} cells into Rag.Ldlr recipients elicited enhanced atherosclerosis compared to T_{N} and TEM cells. Nevertheless, adoptive transfer of IFN-γ-deficient CD8 T cells does not increase atherosclerotic lesion formation, arguing that additional CD8⁺ T cell-specific mechanisms are responsible for inducing atherosclerosis 6. This disclosure demonstrates that the T_{SCM} cell is an essential component of the CD8⁺ T cell compartment in heightening atherosclerosis disease severity.

CD8⁺ T_{SCM} cells adoptive transfer elicited increased atherosclerotic lesions compared to CD8⁺ TEM cells. TEM cells, characterized by high expression of CD44 and the absence of the lymph node homing receptor CD62L, are maintained and endowed with memory against subsequent, repeated attacks by an invading pathogen. TEM cells migrate to sites of inflammation via the chemokine receptors CCR5 and CXCR3 and are noted by increased expression of the pro-inflammatory mediators perforin and granzyme.

CD8⁺ T_{SCM} cells have demonstrated potent anti-tumor killing and represent a powerful candidate for adoptive T cell therapy for cancer; in particular, transferred T_{SCM} cells more efficiently killed tumor cells compared to T_{CM} or T_{EM} cells, eliciting significantly reduced tumor burden and increased survival of tumor-bearing mice³¹. Similar to anti-tumor killing, perhaps these cells, are exquisitely atherogenic due to production of the pro-atherogenic cytokine IFN-γ, increased proliferative capacity, and ability to rapidly differentiate into T_{CM} and T_{EM} subsets. Future studies will examine the mechanism by which T_{SCM} cells are exquisitely pro-atherogenic.

Cardiovascular disease (CVD) remains a major health concern, in which diagnosis requires invasive methods, including cardiac catheterization, employed to visualize blood flow through the heart. In the United States, more than 1 million cardiac catheterizations are performed annually⁴⁰ and carry numerous risks, including allergic reaction to the injected dye, stroke, heart attack, and blood clotting⁴¹. The discovery of an indicator of CVD severity that can be detected in the peripheral blood would vastly improve current detection methods. The inventors have shown that CD95 expression on CD8⁺ T_{N} cells could constitute an improved diagnostic technique to more quickly and easily diagnose CVD risk, requiring less than 1 mL of whole blood.

In summary, the inventors observed significantly reduced CD8⁺ T_{N} cell frequencies in the peripheral blood of individuals with high compared to low CVD. Examination of the CD8⁺ T_{N} compartment revealed a significant increase in the memory antigen, CD95, a well-established marker of T_{SCM} cells. Investigating T_{SCM} cells using previously defined gating strategies revealed a robust enhancement in high CVD compared to low CVD individuals. These results demonstrate that CD95 expression on CD8⁺ T_{N} cells may serve as a novel biomarker to accurately predict CVD risk.

Thus, in particular embodiments, the elements of the present invention may decrease, reduce, inhibit, suppress or disrupt an immune or inflammatory response. In still further embodiments, the elements of the present invention may elicit, stimulate, induce, promote, increase or enhance an anti-immune or anti-inflammatory response.

The elements of the present invention can be employed in various methods, uses and compositions. Such methods and uses include, for example, use, contact or administration of one or more elements of the present invention in vitro and in vivo. Such methods are applicable to providing treatment to a subject for an adverse cardiovascular event or cardiovascular disease (e.g. atherosclerosis).

In another aspect of the present invention there are provided methods for treatment of an adverse cardiovascular event or cardiovascular disease (e.g. atherosclerosis). Thus, in one embodiment of the present invention there is provided a method of treating a subject for an adverse cardiovascular event or cardiovascular disease as disclosed herein, sufficient to treat the subject for the adverse cardiovascular event or cardiovascular disease.

As will be understood by a person skilled in the art, treating a subject for an adverse cardiovascular event or cardiovascular disease may include decreasing, reducing, inhibiting, suppressing, limiting, controlling or eliminating an adverse cardiovascular event or cardiovascular disease. In other embodiments, a method of treating a subject for an adverse cardiovascular event or cardiovascular disease comprises reducing the frequency, severity, progression, or duration of the adverse cardiovascular event or cardiovascular disease in the subject. In yet another embodiment, a method of treating a subject for an adverse cardiovascular event or cardiovascular disease comprises maintaining the severity of an adverse cardiovascular event or cardiovascular disease in a subject by preventing an increase in the occurrence, frequency, severity, progression, or duration of the adverse cardiovascular event or cardiovascular disease in the subject. In still further embodiments, a method of treating a subject for an adverse cardiovascular event or cardiovascular disease comprises eliminating, reducing or maintaining the occurrence, frequency, severity, progression, or duration of physiological conditions, disorders, illnesses, diseases, symptoms or complications caused by or associated with the adverse cardiovascular event or cardiovascular disease.

In certain embodiments, the subject of the methods provided herein may have previously had an adverse cardiovascular event or cardiovascular disease. Thus, in certain embodiments, the present methods may be used for treating or protecting a subject from a secondary or subsequent adverse cardiovascular event or cardiovascular disease. Thus, in different embodiments, the presently described methods of treatment may be used for prophylactic treatment of an adverse cardiovascular event or cardiovascular disease or can be used treat a secondary or subsequent occurrence of an adverse cardiovascular event or cardiovascular disease. In particular embodiments, the methods may be used to treat an adverse symptom of the adverse cardiovascular event or cardiovascular disease.

In accordance with different embodiments of the present invention, the therapeutic and prophylactic methods of treating a subject for an adverse cardiovascular event or cardiovascular disease include but are not limited to treatment of a subject having or at risk of having an adverse cardiovascular event or cardiovascular disease, treating a subject with an adverse cardiovascular event or cardiovascular disease, and methods of protecting a subject from an adverse cardiovascular event or cardiovascular disease (e.g., provide the subject with protection against the development or incidence of an adverse cardiovascular event or cardiovascular disease), to decrease or reduce the probability of an adverse cardiovascular event or cardiovascular disease in a subject, to decrease or reduce susceptibility of a subject to an adverse cardiovascular event or cardiovascular disease and to inhibit or prevent an adverse cardiovascular event or cardiovascular disease in a subject. In particular embodiments of the methods described herein, one or more disorders, diseases, physiological conditions, pathologies and symptoms associated with or caused by an adverse cardiovascular event or cardiovascular disease will respond to treatment.

Non-limiting examples of an adverse cardiovascular event or cardiovascular disease are atherosclerosis, coronary artery disease, peripheral artery disease, cerebrovascular disease, renal artery disease, stroke, myocardial infarction (heart attack), ischemic heart failure, transient ischemic attack or brain trauma, artherosclerotic plaque formation, foam cells or foam cell formation, or one or more adverse symptoms, disorders, illnesses, pathologies, diseases, or complications caused by or associated with an adverse cardiovascular event or cardiovascular disease such as atherosclerosis, coronary artery disease, peripheral artery disease, cerebrovascular disease, renal artery disease, stroke, myocardial infarction (heart attack), ischemic heart failure, transient ischemic attack or brain trauma), artherosclerotic plaque formation or foam cells or foam cell formation.

Methods of the invention include methods treatment that result in any therapeutic or beneficial effect. In various methods embodiments, an adverse cardiovascular event or cardiovascular disease is reduced, decreased, inhibited, limited, delayed or prevented, or a method decreases, reduces, inhibits, suppresses, prevents, controls or limits one or more adverse (e.g., physical) symptoms, disorders, illnesses, diseases or complications caused by or associated with adverse cardiovascular event or cardiovascular disease. In additional various particular embodiments, methods of the present invention include reducing, decreasing, inhibiting, delaying or preventing onset, progression, frequency, duration, severity, probability or susceptibility of one or more adverse symptoms, disorders, illnesses, diseases or complications caused by or associated with an adverse cardiovascular event or cardiovascular disease. In further various particular embodiments, methods of the present invention include improving, accelerating, facilitating, enhancing, augmenting, or hastening recovery of a subject from an adverse cardiovascular event or cardiovascular disease, or one or more adverse symptoms, disorders, illnesses, diseases or complications caused by or associated with an adverse cardiovascular event or cardiovascular disease. In yet additional various embodiments, methods of treatment include stabilizing an adverse cardiovascular event or cardiovascular disease, or an adverse symptom, disorder, illness, disease or complication caused by or associated with an adverse cardiovascular event or cardiovascular disease.

A therapeutic or beneficial effect of treatment is therefore any objective or subjective measurable or detectable improvement or benefit provided to a particular subject. A therapeutic or beneficial effect can but need not be complete ablation of or protection from all or any particular adverse symptom, disorder, illness, disease or complication caused by or associated with adverse cardiovascular event or cardiovascular disease Thus, a satisfactory clinical endpoint is achieved when there is an incremental improvement or a partial reduction in an adverse symptom, disorder, illness, disease or complication caused by or associated with an adverse cardiovascular event or cardiovascular disease or an inhibition, decrease, reduction, suppression, prevention, limit or control of worsening or progression of one or more adverse symptoms, disorders, illnesses, diseases or complications caused by or associated with an adverse cardiovascular event or cardiovascular disease over a short or long duration (hours, days, weeks, months, etc.).

A therapeutic or beneficial effect also includes reducing or eliminating the need, dosage frequency or amount of a second active such as another drug or other agent used for treating a subject having or at risk of having an adverse cardiovascular event or cardiovascular disease For example, reducing an amount of an adjunct therapy, for example, a reduction or decrease of a treatment for an adverse cardiovascular event or cardiovascular disease, or a vaccination or immunization protocol for an adverse cardiovascular event or cardiovascular disease is considered a beneficial effect.

Methods and compositions of the invention include administration the diagnostics, treatments, and agents disclosed herein to a subject prior to development of an adverse cardiovascular event or cardiovascular disease, administration substantially contemporaneously with development of an adverse cardiovascular event or cardiovascular disease, and administration after development of an adverse cardiovascular event or cardiovascular disease. Methods and compositions of the invention also include administration of the diagnostics, treatments, and agents disclosed herein to a subject prior to, substantially contemporaneously with or following development of an adverse symptom, disorder, illness or disease caused by or associated with an adverse cardiovascular event or cardiovascular disease. A subject suffering from an adverse cardiovascular event or cardiovascular disease or an adverse symptom, disorder, illness or disease caused by or associated with an adverse cardiovascular event or cardiovascular disease have the adverse cardiovascular event, cardiovascular disease or symptom over a period of 1-5, 5-10, 10-20, 20-30, 30-50, 50-100 hours, days, months, or years.

Methods and compositions of the invention include administration of the diagnostics, treatments, and agents disclosed herein, to a subject alone or in combination with any compound, agent, drug, treatment or other therapeutic regimen or protocol having a desired therapeutic, beneficial, additive, synergistic or complementary activity or effect.

Exemplary combination compositions and treatments include the diagnostics, treatments, and agents disclosed herein, and second actives, such as compounds, agents, drugs, treatments and therapies for the treatment of an adverse cardiovascular event or cardiovascular disease (e.g. statins, fibrate), as well as agents that assist, promote, stimulate or enhance efficacy. Such compounds, agents, drugs, treatments and therapies can be administered or performed prior to, substantially contemporaneously with or following any composition or method of the invention, for example, a therapeutic use or method of treating a subject for an adverse cardiovascular event or cardiovascular disease (e.g. atherosclerosis), or a method of prophylactic treatment of a subject for an adverse cardiovascular event or cardiovascular disease (e.g. atherosclerosis).

Invention compositions comprise the diagnostics, treatments, and agents disclosed herein, and methods described herein can be combined with any compound, agent, drug, treatment or other therapeutic regimen or protocol having a desired therapeutic, beneficial, additive, synergistic or complementary activity or effect. Exemplary combination compositions and treatments include elements disclosed herein and second actives, such as compounds, agents, drugs, treatments and therapies for the treatment of an adverse cardiovascular event or cardiovascular disease (e.g. statins, fibrate), as well as agents that assist, promote, stimulate or enhance efficacy. Such compounds, agents, drugs, treatments and therapies can be administered or performed prior to, substantially contemporaneously with or following any composition or method of the invention, for example, a therapeutic use or method of treating a subject for an adverse cardiovascular event or cardiovascular disease (e.g. atherosclerosis), or a method of prophylactic treatment of a subject for an adverse cardiovascular event or cardiovascular disease (e.g. atherosclerosis).

The invention therefore provides treatments in combination with a second active, including but not limited to any compound, agent, drug, therapeutic regimen, treatment protocol, process, remedy or composition, such as a treatment protocol set forth herein or known in the art. The compound, agent, drug, therapeutic regimen, treatment protocol, process, remedy or composition can be administered or performed prior to, substantially contemporaneously with or following administration of elements disclosed herein to a subject. Specific non-limiting examples of combination embodiments therefore include the foregoing or other compound, agent, drug, therapeutic regimen, treatment protocol, process, remedy or composition.

In methods of the present invention, compositions are used for which there is a desired outcome, such as a therapeutic or prophylactic method that provides a benefit from treatment, vaccination or immunization, and can be administered in a sufficient or effective amount.

As used herein, a “sufficient amount” or “effective amount” or an “amount sufficient” or an “amount effective” refers to an amount that provides, in single (e.g., primary) or multiple (e.g., booster) doses, alone or in combination with one or more other compounds, treatments, therapeutic regimens or agents (e.g., a drug), a long term or a short term detectable or measurable improvement in a given subject or any objective or subjective benefit to a given subject of any degree or for any time period or duration (e.g., for minutes, hours, days, months, years, or cured).

An amount sufficient or an amount effective can but need not be provided in a single administration and can but need not be achieved by elements disclosed herein alone, but optionally in a combination composition or method that includes a second active. In addition, an amount sufficient or an amount effective need not be sufficient or effective if given in single or multiple doses without a second or additional administration or dosage, since additional doses, amounts or duration above and beyond such doses, or additional antigens, compounds, drugs, agents, treatment or therapeutic regimens may be included in order to provide a given subject with a detectable or measurable improvement or benefit to the subject.

An amount sufficient or an amount effective need not be therapeutically or prophylactically effective in each and every subject treated, nor a majority of subjects treated in a given group or population. An amount sufficient or an amount effective means sufficiency or effectiveness in a particular subject, not a group of subjects or the general population. As is typical for such methods, different subjects will exhibit varied responses to a method of the invention, such as vaccination and therapeutic treatments.

The term “subject” refers includes but is not limited to a subject at risk of an adverse cardiovascular event or cardiovascular disease (e.g. atherosclerosis), as well as a subject that has already developed an adverse cardiovascular event or cardiovascular disease (e.g. atherosclerosis).

Such subjects, include mammalian animals (mammals), such as a non-human primate (apes, gibbons, gorillas, chimpanzees, orangutans, macaques), a domestic animal (dogs and cats), a farm animal (poultry such as chickens and ducks, horses, cows, goats, sheep, pigs), experimental animal (mouse, rat, rabbit, guinea pig) and humans. Subjects include animal disease models, for example, mouse and other animal models of an adverse cardiovascular event or cardiovascular disease (e.g. atherosclerosis) known in the art.

Accordingly, subjects appropriate for treatment include those having or at risk of an adverse cardiovascular event or cardiovascular disease (e.g. atherosclerosis), also referred to as subjects in need of treatment. Subjects in need of treatment therefore include subjects that have been previously had an adverse cardiovascular event or cardiovascular disease (e.g. atherosclerosis) or that have an ongoing adverse cardiovascular event or cardiovascular disease (e.g. atherosclerosis) or have developed one or more adverse symptoms caused by or associated with an adverse cardiovascular event or cardiovascular disease (e.g. atherosclerosis), regardless of the type, timing or degree of onset, progression, severity, frequency, duration of the symptoms.

Prophylactic uses and methods are therefore included. Target subjects for prophylaxis may be at increased risk (probability or susceptibility) of developing an adverse cardiovascular event or cardiovascular disease (e.g. atherosclerosis). Such subjects are considered in need of treatment due to being at risk.

Subjects for prophylaxis need not be at increased risk but may be from the general population in which it is desired to protect a subject against an adverse cardiovascular event or cardiovascular disease (e.g. atherosclerosis), for example. Such a subject that is desired to be protected against an adverse cardiovascular event or cardiovascular disease (e.g. atherosclerosis) can be administered treatment or agent described herein. In another non-limiting example, a subject that is not specifically at risk for an adverse cardiovascular event or cardiovascular disease (e.g. atherosclerosis), but nevertheless desires protection against an adverse cardiovascular event or cardiovascular disease (e.g. atherosclerosis), can be administered treatment or an agent described herein. Such subjects are also considered in need of treatment.

“Prophylaxis” and grammatical variations thereof mean a method in which contact, administration or in vivo delivery to a subject is prior to development of an adverse cardiovascular event or cardiovascular disease. In certain situations it may not be known that a subject has developed an adverse cardiovascular event or cardiovascular disease, but administration or in vivo delivery to a subject can be performed prior to manifestation of disease pathology or an associated adverse symptom, condition, complication, etc. caused by or associated with an adverse cardiovascular event or cardiovascular disease. In such case, a composition or method of the present invention can eliminate, prevent, inhibit, suppress, limit, decrease or reduce the probability of or susceptibility to an adverse cardiovascular event or cardiovascular, or an adverse symptom, condition or complication associated with or caused by an adverse cardiovascular event or cardiovascular disease.

“Prophylaxis” can also refer to a method in which contact, administration or in vivo delivery to a subject is prior to a secondary or subsequent exposure or infection. In such a situation, a subject may have had a prior adverse cardiovascular event or cardiovascular disease or prior adverse symptom, condition or complication associated with or caused by an adverse cardiovascular event or cardiovascular disease. Treatment by administration or in vivo delivery to such a subject, can be performed prior to a secondary or subsequent adverse cardiovascular event or cardiovascular disease. Such a method can eliminate, prevent, inhibit, suppress, limit, decrease or reduce the probability of or susceptibility towards a secondary or subsequent adverse cardiovascular event or cardiovascular disease, or an adverse symptom, condition or complication associated with or caused by or associated with a secondary or subsequent adverse cardiovascular event or cardiovascular disease.

Treatment of an adverse cardiovascular event or cardiovascular disease can be at any time during the adverse cardiovascular event or cardiovascular disease. Certain embodiments of the present invention can be administered as a combination (e.g., with a second active), or separately concurrently or in sequence (sequentially) in accordance with the methods described herein as a single or multiple dose e.g., one or more times hourly, daily, weekly, monthly or annually or between about 1 to 10 weeks, or for as long as appropriate, for example, to achieve a reduction in the onset, progression, severity, frequency, duration of one or more symptoms or complications associated with or caused by an adverse cardiovascular event or cardiovascular disease (e.g. atherosclerosis), or an adverse symptom, condition or complication associated with or caused by an adverse cardiovascular event or cardiovascular disease (e.g. atherosclerosis). Thus, a method can be practiced one or more times (e.g., 1-10, 1-5 or 1-3 times) an hour, day, week, month, or year. The skilled artisan will know when it is appropriate to delay or discontinue administration. A non-limiting dosage schedule is 1-7 times per week, for 1, 2, 3, 4, 5, 6, 7, 8, 9, 10, 15, 20 or more weeks, and any numerical value or range or value within such ranges.

Methods of the invention may be practiced by any mode of administration or delivery, or by any route, systemic, regional and local administration or delivery. Exemplary administration and delivery routes include intravenous (i.v.), intraperitoneal (i.p.), intrarterial, intramuscular, parenteral, subcutaneous, intra-pleural, topical, dermal, intradermal, transdermal, transmucosal, intra-cranial, intra-spinal, rectal, oral (alimentary), mucosal, inhalation, respiration, intranasal, intubation, intrapulmonary, intrapulmonary instillation, buccal, sublingual, intravascular, intrathecal, intracavity, iontophoretic, intraocular, ophthalmic, optical, intraglandular, intraorgan, or intralymphatic.

Doses can be based upon current existing protocols, empirically determined, using animal disease models or optionally in human clinical trials. Initial study doses can be based upon animal studies, e.g. a mouse, and the treatment or agent described herein, administered as is determined to be effective. Exemplary non-limiting amounts (doses) are in a range of about 0.1 mg/kg to about 100 mg/kg, and any numerical value or range or value within such ranges. Greater or lesser amounts (doses) can be administered, for example, 0.01-500 mg/kg, and any numerical value or range or value within such ranges. The dose can be adjusted according to the mass of a subject, and will generally be in a range from about 1-10 ug/kg, 10-25 ug/kg, 25-50 ug/kg, 50-100 ug/kg, 100-500 ug/kg, 500-1,000 ug/kg, 1-5 mg/kg, 5-10 mg/kg, 10-20 mg/kg, 20-50 mg/kg, 50-100 mg/kg, 100-250 mg/kg, 250-500 mg/kg, or more, two, three, four, or more times per hour, day, week, month or annually. A typical range will be from about 0.3 mg/kg to about 50 mg/kg, 0-25 mg/kg, or 1.0-10 mg/kg, or any numerical value or range or value within such ranges.

Doses can vary and depend upon whether the treatment is prophylactic or therapeutic, whether a subject has previously had an adverse cardiovascular event or cardiovascular disease, the onset, progression, severity, frequency, duration probability of or susceptibility of the symptom, condition, pathology or complication, the treatment protocol and compositions, the clinical endpoint desired, the occurrence of previous or simultaneous treatments, the general health, age, gender, race or immunological competency of the subject and other factors that will be appreciated by the skilled artisan. The skilled artisan will appreciate the factors that may influence the dosage and timing required to provide an amount sufficient for providing a therapeutic or prophylactic benefit.

The dose amount, number, frequency or duration may be proportionally increased or reduced, as indicated by the status of the subject. For example, whether the subject has previously had an adverse cardiovascular event or cardiovascular disease, whether the subject is merely at risk of an adverse cardiovascular event or cardiovascular disease, exposure or infection, whether the subject has been previously treated for an adverse cardiovascular event or cardiovascular disease. The dose amount, number, frequency or duration may be proportionally increased or reduced, as indicated by any adverse side effects, complications or other risk factors of the treatment or therapy.

In the methods of the invention, the route, dose, number and frequency of administrations, treatments, and timing/intervals between treatment and disease development can be modified. In certain embodiments, a desirable treatment of the present invention will elicit robust, long-lasting immunity against an adverse cardiovascular event or cardiovascular disease. Thus, in certain embodiments, invention methods, uses and compositions provide long-lasting immunity to an adverse cardiovascular event or cardiovascular disease such as atherosclerosis.

Certain embodiments of the present invention may be provided as pharmaceutical compositions.

In certain embodiment, the present invention includes a method of preventing or treating a patient with an increased risk of developing a cardiovascular disease or has existing cardiovascular disease, comprising: determining a ratio of T_{scm} to T_{N} from a biological sample obtained from the patient, wherein a higher ratio of T_{scm} to T_{N} in the biological sample compared to the ratio of T_{scm} to T_{N} in a healthy patient or CVDlo patient indicates the patient is in need of treatment to prevent or treat the cardiovascular disease; and treating the patient with a T_{scm} modulating therapy. A T_{scm}, Tn, or T_{CD25} modulating therapy is a T_{scm} depleting therapy, a T_{N} increasing therapy, or a T_{CD25}CD8+CD3+CD25+ T cell therapy. The therapy for the prevention or treatment of the cardiovascular disease is administered systemically, regionally or locally, via ingestion, via inhalation, topically, intravenously, or orally. The therapy for the prevention or treatment of the cardiovascular disease is adoptive T cell therapy, chimeric antigen receptor T cell (CAR-T) therapy, antibody dependent cell cytotoxicity therapy, antibody drug conjugate therapy, T-cell receptor (TCR) modified T-cell therapy, chimeric antigen receptor (CAR)-modified natural killer cell therapy, administration of monoclonal antibodies, administration of cytokines, T cell suppressing therapy, or any combination thereof.

As used herein the term “pharmaceutically acceptable” and “physiologically acceptable” mean a biologically acceptable formulation, gaseous, liquid or solid, or mixture thereof, which is suitable for one or more routes of administration, in vivo delivery or contact. Such formulations include solvents (aqueous or non-aqueous), solutions (aqueous or non-aqueous), emulsions (e.g., oil-in-water or water-in-oil), suspensions, syrups, elixirs, dispersion and suspension media, coatings, isotonic and absorption promoting or delaying agents, compatible with pharmaceutical administration or in vivo contact or delivery. Aqueous and non-aqueous solvents, solutions and suspensions may include suspending agents and thickening agents. Such pharmaceutically acceptable carriers include tablets (coated or uncoated), capsules (hard or soft), microbeads, powder, granules and crystals. Supplementary active compounds (e.g., preservatives, antibacterial, antiviral and antifungal agents) can also be incorporated into the compositions.

Pharmaceutical compositions can be formulated to be compatible with a particular route of administration. Thus, pharmaceutical compositions include carriers, diluents, or excipients suitable for administration by various routes. Exemplary routes of administration for contact or in vivo delivery which a composition can optionally be formulated include inhalation, respiration, intranasal, intubation, intrapulmonary instillation, oral, buccal, intrapulmonary, intradermal, topical, dermal, parenteral, sublingual, subcutaneous, intravascular, intrathecal, intraarticular, intracavity, transdermal, iontophoretic, intraocular, opthalmic, optical, intravenous (i.v.), intramuscular, intraglandular, intraorgan, or intralymphatic.

Formulations suitable for parenteral administration comprise aqueous and non-aqueous solutions, suspensions or emulsions of the active compound, which preparations are typically sterile and can be isotonic with the blood of the intended recipient. Non-limiting illustrative examples include water, saline, dextrose, fructose, ethanol, animal, vegetable or synthetic oils.

To increase a treatment as described herein comprising a vaccination, a composition of the present invention can be coupled to one or more proteins such as ovalbumin or keyhole limpet hemocyanin (KLH), thyroglobulin or a toxin such as tetanus or cholera toxin. Invention compositions can also be mixed with adjuvants. As demonstrated herein, in certain embodiments, the form of adjuvant with which the invention proteins or peptides are mixed may change whether the protein or peptide elicits an atherogenic or protective response in a subject.

Adjuvants include, for example: Oil (mineral or organic) emulsion adjuvants such as Freund's complete (CFA) and incomplete adjuvant (IFA) (WO 95/17210; WO 98/56414; WO 99/12565; WO 99/11241; and U.S. Pat. No. 5,422,109); metal and metallic salts, such as aluminum and aluminum salts, such as aluminum phosphate or aluminum hydroxide, alum (hydrated potassium aluminum sulfate); bacterially derived compounds, such as Monophosphoryl lipid A and derivatives thereof (e.g., 3 De-O-acylated monophosphoryl lipid A, aka 3D-MPL or d3-MPL, to indicate that position 3 of the reducing end glucosamine is de-O-acylated, 3D-MPL consisting of the tri and tetra acyl congeners), and enterobacterial lipopolysaccharides (LPS); plant derived saponins and derivatives thereof, for example Quil A (isolated from the Quilaja Saponaria Molina tree, see, e.g., “Saponin adjuvants”, Archiv. fur die gesamte Virusforschung, Vol. 44, Springer Verlag, Berlin, p243-254; U.S. Pat. No. 5,057,540), and fragments of Quil A which retain adjuvant activity without associated toxicity, for example QS7 and QS21 (also known as QA7 and QA21), as described in WO96/33739, for example; surfactants such as, soya lecithin and oleic acid; sorbitan esters such as sorbitan trioleate; and polyvinylpyrrolidone; oligonucleotides such as CpG (WO 96/02555, and WO 98/16247), polyriboA and polyriboU; block copolymers; and immunostimulatory cytokines such as GM-CSF and IL-1, and Muramyl tripeptide (MTP). Additional examples of adjuvants are described, for example, in “Vaccine Design—the subunit and adjuvant approach” (Edited by Powell, M. F. and Newman, M. J.; 1995, Pharmaceutical Biotechnology (Plenum Press, New York and London, ISBN 0-306-44867-X) entitled “Compendium of vaccine adjuvants and excipients” by Powell, M. F. and Newman M.

Salts may be added to a composition of the present invention. Non-limiting examples of salts include acetate, benzoate, besylate, bitartate, bromide, carbonate, chloride, citrate, edetate, edisylate, estolate, fumarate, gluceptate, gluconate, hydrobromide, hydrochloride, iodide, lactate, lactobionate, malate, maleate, mandelate, mesylate, methyl bromide, methyl sulphate, mucate, napsylate, nitrate, pamoate (embonate, phosphate, diphosphate, salicylate and disalicylate, stearate, succinate, sulphate, tartrate, tosylate, triethiodide, valerate, aluminium, benzathine, calcium, ethylene diamine, lysine, magnesium, megluminie, potassium, procaine, sodium, tromethyamine or zinc.

Chelating agents may be added to a composition of the present invention. Non-limiting examples of chelating agents include ethylenediamine, ethylene glycol tetraacetic acid, 1,2-bis(o-aminophenoxy)ethane-N,N,N′,N′-tetraacetic acid, Penicillamine, Deferasirox, Deferiprone, Deferoxamine, 2,3-Disulfanylpropan-1-ol, Dexrazoxane, Iron(II,III) hexacyanoferrate(II,III), (R)-5-(1,2-dithiolan-3-yl)pentanoic acid, 2,3-Dimercapto-1-propanesulfonic acid, Dimercaptosuccinic acid, or diethylene triamine pentaacetic acid.

Buffering agents may be added to a composition of the present invention. Non-limiting examples of buffering agents include phosphate, citrate, acetate, borate, TAPS, bicine, tris, tricine, TAPSO, HEPES, TES, MOPS, PIPES, cacodylate, SSC, MES or succinic acid.

Cosolvents may be added to a composition of the present invention. Non-limiting examples of cosolvents contain hydroxyl groups or other polar groups, for example, alcohols, such as isopropyl alcohol; glycols, such as propylene glycol, polyethyleneglycol, polypropylene glycol, glycol ether; glycerol; polyoxyethylene alcohols and polyoxyethylene fatty acid esters. Non-limiting examples of cosolvents contain hydroxyl groups or other polar groups, for example, alcohols, such as isopropyl alcohol; glycols, such as propylene glycol, polyethyleneglycol, polypropylene glycol, glycol ether; glycerol; polyoxyethylene alcohols and polyoxyethylene fatty acid esters.

Supplementary compounds (e.g., preservatives, antioxidants, antimicrobial agents including biocides and biostats such as antibacterial, antiviral and antifungal agents) can also be incorporated into the compositions. Pharmaceutical compositions may therefore include preservatives, anti-oxidants and antimicrobial agents.

Preservatives can be used to inhibit microbial growth or increase stability of ingredients thereby prolonging the shelf life of the pharmaceutical formulation. Suitable preservatives are known in the art and include, for example, EDTA, EGTA, benzalkonium chloride or benzoic acid or benzoates, such as sodium benzoate. Antioxidants include, for example, ascorbic acid, vitamin A, vitamin E, tocopherols, and similar vitamins or provitamins.

An antimicrobial agent or compound directly or indirectly inhibits, reduces, delays, halts, eliminates, arrests, suppresses or prevents contamination by or growth, infectivity, replication, proliferation, reproduction, of a pathogenic or non-pathogenic microbial organism. Classes of antimicrobials include antibacterial, antiviral, antifungal and antiparasitics. Antimicrobials include agents and compounds that kill or destroy (-cidal) or inhibit (-static) contamination by or growth, infectivity, replication, proliferation, reproduction of the microbial organism.

Exemplary antibacterials (antibiotics) include penicillins (e.g., penicillin G, ampicillin, methicillin, oxacillin, and amoxicillin), cephalosporins (e.g., cefadroxil, ceforanid, cefotaxime, and ceftriaxone), tetracyclines (e.g., doxycycline, chlortetracycline, minocycline, and tetracycline), aminoglycosides (e.g., amikacin, gentamycin, kanamycin, neomycin, streptomycin, netilmicin, paromomycin and tobramycin), macrolides (e.g., azithromycin, clarithromycin, and erythromycin), fluoroquinolones (e.g., ciprofloxacin, lomefloxacin, and norfloxacin), and other antibiotics including chloramphenicol, clindamycin, cycloserine, isoniazid, rifampin, vancomycin, aztreonam, clavulanic acid, imipenem, polymyxin, bacitracin, amphotericin and nystatin.

Particular non-limiting classes of anti-virals include reverse transcriptase inhibitors; protease inhibitors; thymidine kinase inhibitors; sugar or glycoprotein synthesis inhibitors; structural protein synthesis inhibitors; nucleoside analogues; and viral maturation inhibitors. Specific non-limiting examples of anti-virals include nevirapine, delavirdine, efavirenz, saquinavir, ritonavir, indinavir, nelfinavir, amprenavir, zidovudine (AZT), stavudine (d4T), larnivudine (3TC), didanosine (DDI), zalcitabine (ddC), abacavir, acyclovir, penciclovir, ribavirin, valacyclovir, ganciclovir, 1,-D-ribofuranosyl-1,2,4-triazole-3 carboxamide, 9->2-hydroxy-ethoxy methylguanine, adamantanamine, 5-iodo-2′-deoxyuridine, trifluorothymidine, interferon and adenine arabinoside.

Pharmaceutical formulations and delivery systems appropriate for the compositions and methods of the invention are known in the art (see, e.g., Remington: The Science and Practice of Pharmacy (2003) 20^{th} ed., Mack Publishing Co., Easton, Pa.; Remington's Pharmaceutical Sciences (1990) 18^{th} ed., Mack Publishing Co., Easton, Pa.; The Merck Index (1996) 12^{th} ed., Merck Publishing Group, Whitehouse, N.J.; Pharmaceutical Principles of Solid Dosage Forms (1993), Technonic Publishing Co., Inc., Lancaster, Pa.; Ansel ad Soklosa, Pharmaceutical Calculations (2001) 11th ed., Lippincott Williams & Wilkins, Baltimore, Md.; and Poznansky et al., Drug Delivery Systems (1980), R. L. Juliano, ed., Oxford, N.Y., pp. 253-315).

An agent as described herein can be packaged in unit dosage form (capsules, tablets, troches, cachets, lozenges) for ease of administration and uniformity of dosage. A “unit dosage form” as used herein refers to physically discrete units suited as unitary dosages for the subject to be treated; each unit containing a predetermined quantity of active ingredient optionally in association with a pharmaceutical carrier (excipient, diluent, vehicle or filling agent) which, when administered in one or more doses, is calculated to produce a desired effect (e.g., prophylactic or therapeutic effect). Unit dosage forms also include, for example, ampules and vials, which may include a composition in a freeze-dried or lyophilized state; a sterile liquid carrier, for example, can be added prior to administration or delivery in vivo. Unit dosage forms additionally include, for example, ampules and vials with liquid compositions disposed therein. Individual unit dosage forms can be included in multi-dose kits or containers. Pharmaceutical formulations can be packaged in single or multiple unit dosage form for ease of administration and uniformity of dosage.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described herein.

All applications, publications, patents and other references, GenBank citations and ATCC citations cited herein are incorporated by reference in their entirety. In case of conflict, the specification, including definitions, will control.

As used herein, the singular forms “a,” “and,” and “the” include plural referents unless the context clearly indicates otherwise.

As used herein, numerical values are often presented in a range format throughout this document. The use of a range format is merely for convenience and brevity and should not be construed as an inflexible limitation on the scope of the invention. Accordingly, the use of a range expressly includes all possible subranges, all individual numerical values within that range, and all numerical values or numerical ranges include integers within such ranges and fractions of the values or the integers within ranges unless the context clearly indicates otherwise. This construction applies regardless of the breadth of the range and in all contexts throughout this patent document. Thus, to illustrate, reference to a range of 90-100% includes 91-99%, 92-98%, 93-95%, 91-98%, 91-97%, 91-96%, 91-95%, 91-94%, 91-93%, and so forth. Reference to a range of 90-100%, includes 91%, 92%, 93%, 94%, 95%, 95%, 97%, etc., as well as 91.1%, 91.2%, 91.3%, 91.4%, 91.5%, etc., 92.1%, 92.2%, 92.3%, 92.4%, 92.5%, etc., and so forth. Reference to a range of 1-5 fold therefore includes 1, 2, 3, 4, 5, 6, 7, 8, 9, 10, 11, 12, 13, 14, 15, 16, 17, 18, 19, 20, fold, etc., as well as 1.1, 1.2, 1.3, 1.4, 1.5, fold, etc., 2.1, 2.2, 2.3, 2.4, 2.5, fold, etc., and so forth. Further, for example, reference to a series of ranges of 2-72 hours, 2-48 hours, 4-24 hours, 4-18 hours and 6-12 hours, includes ranges of 2-6 hours, 2, 12 hours, 2-18 hours, 2-24 hours, etc., and 4-27 hours, 4-48 hours, 4-6 hours, etc.

As also used herein a series of range formats are used throughout this document. The use of a series of ranges includes combinations of the upper and lower ranges to provide a range. Accordingly, a series of ranges include ranges which combine the values of the boundaries of different ranges within the series. This construction applies regardless of the breadth of the range and in all contexts throughout this patent document. Thus, for example, reference to a series of ranges such as 5-10, 10-20, 20-30, 30-40, 40-50, 50-75, 75-100, 100-150, and 150-171, includes ranges such as 5-20, 5-30, 5-40, 5-50, 5-75, 5-100, 5-150, 5-171, and 10-30, 10-40, 10-50, 10-75, 10-100, 10-150, 10-171, and 20-40, 20-50, 20-75, 20-100, 20-150, 20-171, and so forth.

The invention is generally disclosed herein using affirmative language to describe the numerous embodiments and aspects. The invention also specifically includes embodiments in which particular subject matter is excluded, in full or in part, such as substances or materials, method steps and conditions, protocols, procedures, assays or analysis. Thus, even though the invention is generally not expressed herein in terms of what is not included, embodiments and aspects that expressly exclude compositions or method steps are nevertheless disclosed and included in the invention.

A number of embodiments of the invention have been described. Nevertheless, it will be understood that various modifications may be made without departing from the spirit and scope of the invention. Accordingly, the following examples are intended to illustrate but not limit the scope of invention described in the claims.

### Example 1—Loss of Naive CD8⁺ T Cells in Peripheral Blood of Individuals with High CVD

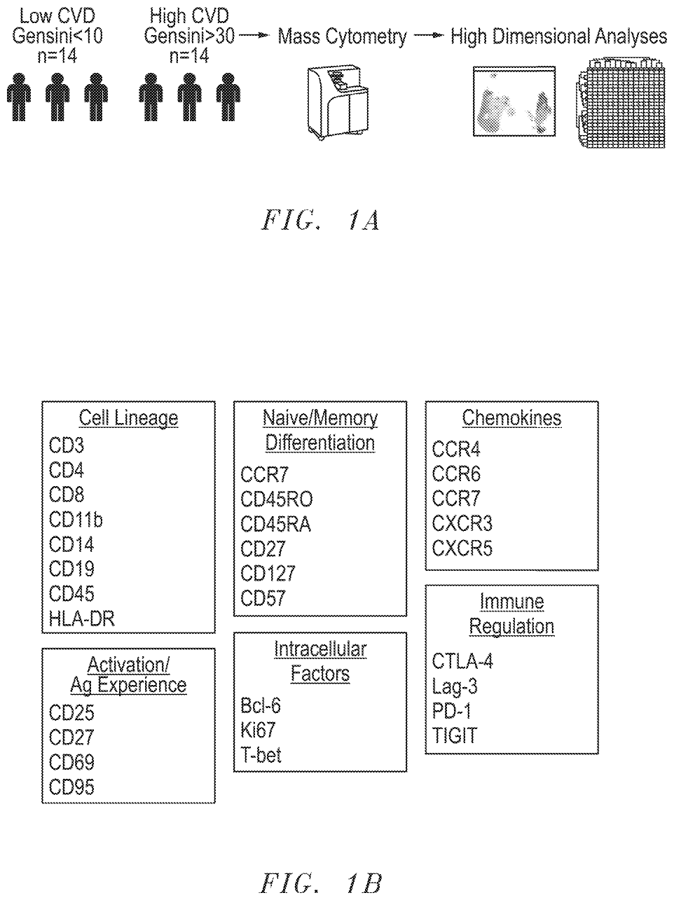

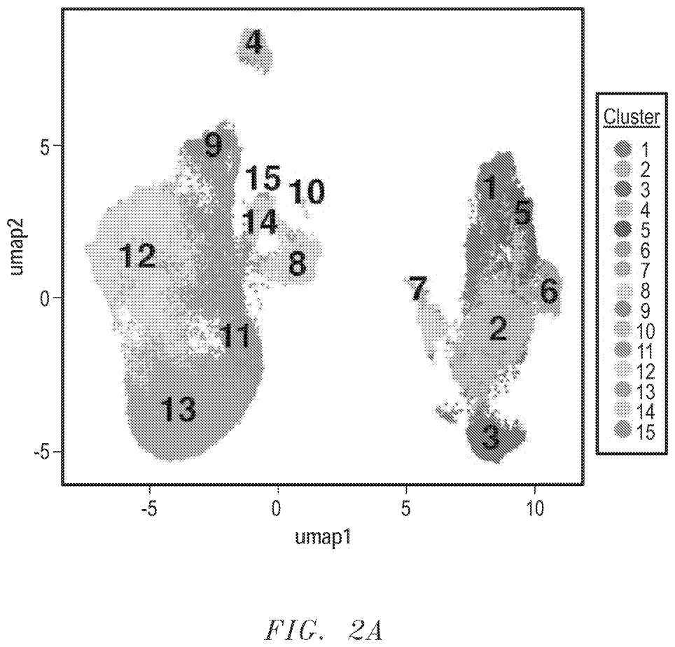

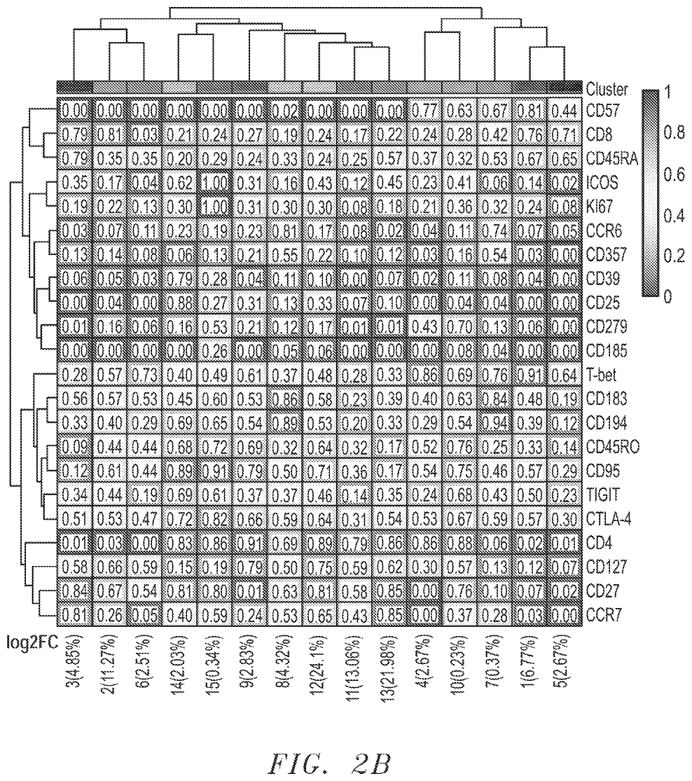

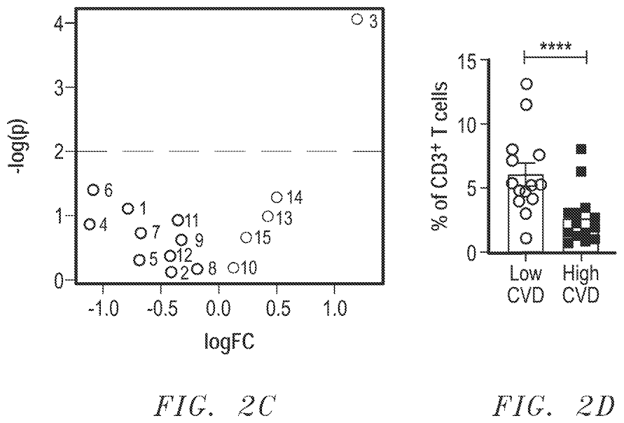

High-dimensional mass cytometry reveals loss of naive CD8⁺ T cells in peripheral blood of individuals with high CVD. To investigate T cell phenotypes that were changed with CVD risk, the inventors obtained PBMCs from 28 matched individuals with either low (n=14) or high (n=14) CVD, based on coronary angiography quantified clinical Gensini scores (FIG. 1A, Table I). The inventors performed mass cytometry on PBMCs from these subjects using a validated, T cell-focused mass cytometry panel comprised of 25 surface antigens and 4 intracellular antigens that consisted of markers of cell lineages, T cell activation, naive/memory T cell differentiation, chemokines, T cell immunoregulation, and transcription factors (FIG. 1B, Table III). As an unbiased strategy for examining T cell heterogeneity, CD3+ T cell populations were clustered via the algorithm Uniform Manifold Approximation and Projection (UMAP), a dimension reduction algorithm that identifies major cell lineages along a broad axis that recapitulates the differentiation state (FIG. 2A). UMAP identified 15 distinct T cell clusters, with 5 T cell clusters attenuated (red circles) and 10 clusters enhanced (blue circles) in high compared to low CVD individuals (FIG. 2B, C).

TABLE I
Individual CVD Characteristics

| | Low | Moderate | High |
|---|---|---|---|
| | Severity | Severity | Severity |
| | CVD | CVD | CVO |
| Variables | (n = 14) | (n = 8) | (n = 14) |
| General Parameters | | | |
| Male (%) | 50% | 71% | 75% |
| Age (Years) | 57.57 ± 10.07 | 68.5 ± 7.91 | 66.07 ± 7.8 |
| Lipids | | | |
| Total Cholesterol (mg/dl) | 164.5 ± 37.36 | 155.4 ± 46.42 | 173.1 ± 53.2 |
| Triglycerides (mg/dl) | 93.9 ± 57.74 | 131.9 ± 68.29 | 132.5 ± 74.64 |
| HDL Cholesterol (mg/dl) | 48.36 ± 17.44 | 40.07 ± 12.97 | 34.88 ± 9.935 |
| LDL Cholesterol (mg/dl) | 46.36 ± 17.44 | 98.5 ± 37.08 | 111.1 ± 45.85 |
| Disease Severity | | | |
| Gensini Score | 3.75 ± 3.577 | 15.88 ± 3.159 | 42.71 ± 9.889 |

Cluster 3 was the most differentially expressed between low and high CVD individuals, and in fact, was the only cluster significantly differentially expressed in abundance following false discovery rate (FDR) correction (FIG. 2C, 2D). Occupying the CD8+ T cell compartment, this population was denoted by high expression of the naive cell markers CD45RA, CCR7, CD127, and CD27, in addition to the absence of the memory marker CD45RO (FIG. 2B). This cluster was significantly attenuated in high compared to low CVD individuals (FIG. 2D).

TABLE II
Healthy donor Characteristics

| Donor | Gender | Age |
|---|---|---|
| Donor 1 | Male | 60/61 |
| Donor 2 | Female | 60/61 |
| Donor 3 | Male | 68/69 |
| Donor 4 | Female | 61/62 |
| Donor 5 | Female | 55/56 |
| Donor 6 | Male | 64/65 |
| Donor 7 | Male | 71/72 |
| Donor 8 | Female | 61/62 |
| Donor 9 | Male | 65/66 |
| Donor 10 | Male | 69/70 |

Notable cell populations that did not reach statistical significance were numbers 13 and 14 (FIG. 2A). Cluster 13 occupied the CD4+ T cell compartment, was attenuated in low compared to high CVD individuals, and similar to cluster 3, exhibited high expression of the naive cell markers CD45RA, CCR7, CD127, and CD27 (FIG. 2B). Circulating CD4+ T_{N} cells were previously shown to inversely correlate with CVD in the Multi-Ethnic Study of Atherosclerosis (MESA). Cluster 14 expressed high levels of the Treg markers CD25, the ectoenzyme CD39, and cytotoxic T lymphocyte-associated protein 4 (CTLA-4), in addition to CD4 and was attenuated in high compared to low CVD (FIG. 2B, 2C). Multiple studies have demonstrated an attenuation in Tregs in the peripheral blood of individuals with CVD 22-24. Interestingly, the less differentiated, naive T cell clusters, noted by expression of CCR7 and CD45RA were situated at the bottom of the UMAP, while more differentiated subsets, expressing CD57, a marker of terminal differentiation, were located at the upper edge, confirming that this algorithm maps the differentiation of T cells.

TABLE III
Online Table III. CyTOP antibodies

| | | Metal | | |
|---|---|---|---|---|
| Antigen | Clone | Conjugate | Manufacturer | Conjugation |
| CD45 | HI30 | 89Y | DVS | DVS |
| CD45 | HI30 | 115In | Biolegend | In-house |
| CCR6 | G034E3 | 141Pr | DVS | DVS |
| CD19 | H1B19 | 142Nd | DVS | DVS |
| CD127 | A019D5 | 143Nd | DVS | DVS |
| CD69 | FN50 | 144Nd | DVS | DVS |
| CD4 | RPA-T4 | 145Nd | DVS | DVS |
| CD8 | RPA-T8 | 146Nd | DVS | DVS |
| CD45 | HI30 | 147Sm | Biolegend | In-house |
| CD14 | RMO52 | 148Nd | DVS | DVS |
| CD45RO | UCHL1 | 149Sm | DVS | DVS |
| CD107a | H4A3 | 151Eu | DVS | DVS |
| EOMES | WD1928 | 152Sm | DVS | DVS |
| CD45RA | 205410 | 153Eu | DVS | DVS |
| CD3 | UCHT1 | 154Sm | DVS | DVS |
| PD-1 | EH12.2H7 | 155Gd | DVS | DVS |
| CXCR3 | G025H7 | 156Gd | DVS | DVS |
| CD27 | L128 | 158Gd | DVS | DVS |
| CD39 | A1 | 160Gd | DVS | DVS |
| T-bet | 4B10 | 161Dy | DVS | DVS |
| GITR | 621 | 162Dy | DVS | DVS |
| Bcl-6 | K11291 | 163Dy | DVS | DVS |
| CD95 | Dx2 | 164Dy | DVS | DVS |
| Foxp3 | PCH101 | 165Ho | ThermoFisher | In-house |
| TIGIT | MBSA43 | 166Er | DVS | DVS |
| CCR7 | G043H7 | 167Er | DVS | DVS |
| ICOS | C398.4A | 168Er | DVS | DVS |
| CD25 | 2A3 | 169Tm | DVS | DVS |
| CTLA-4 | 14D3 | 170Er | DVS | DVS |
| CXCR5 | RF8B | 171Yb | DVS | DVS |
| Ki67 | BL168 | 172Yb | DVS | DVS |
| HLA-DR | L243 | 174Yb | DVS | DVS |
| CCR4 | 205410 | 175Lu | DVS | DVS |
| CD57 | HCD57 | 176Yb | DVS | DVS |
| CD11b | ICRF44 | 209Bi | DVS | DVS |

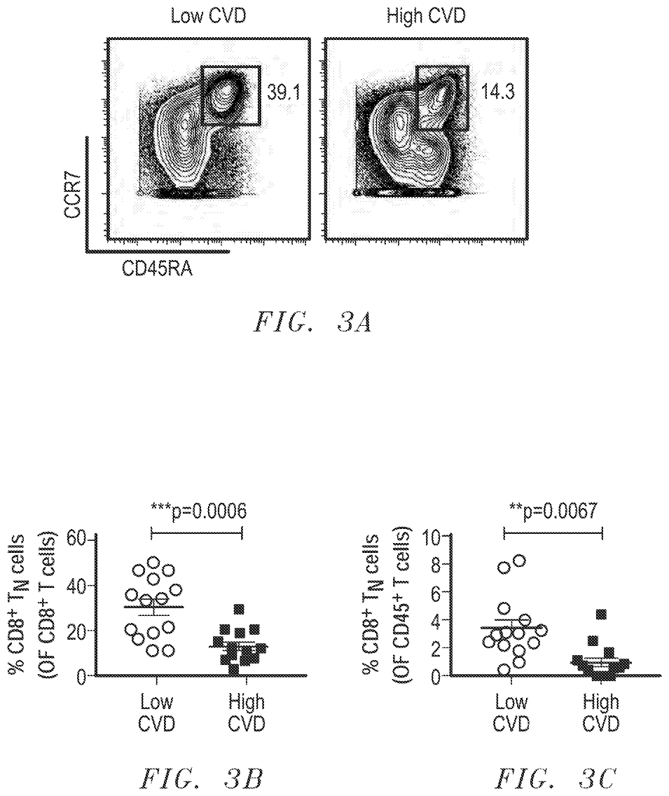

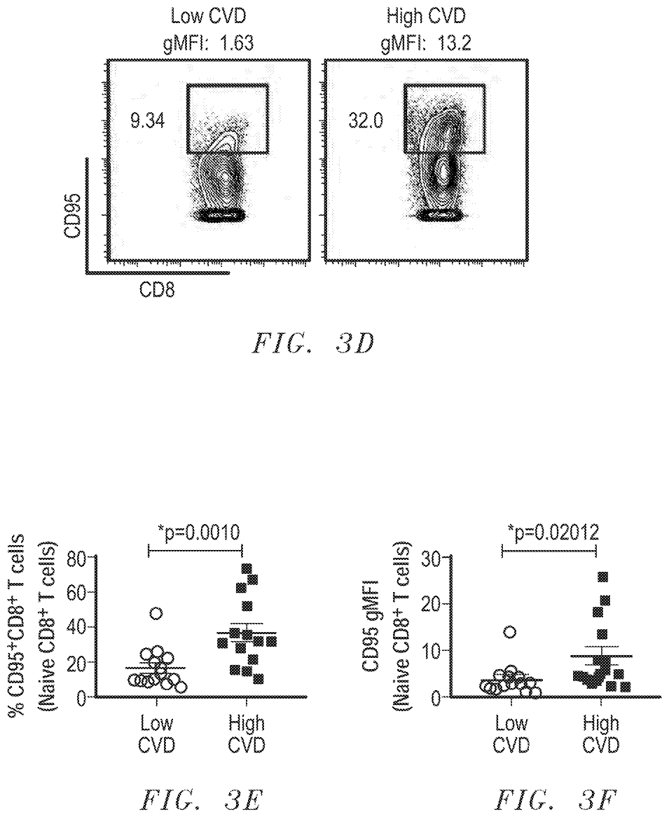

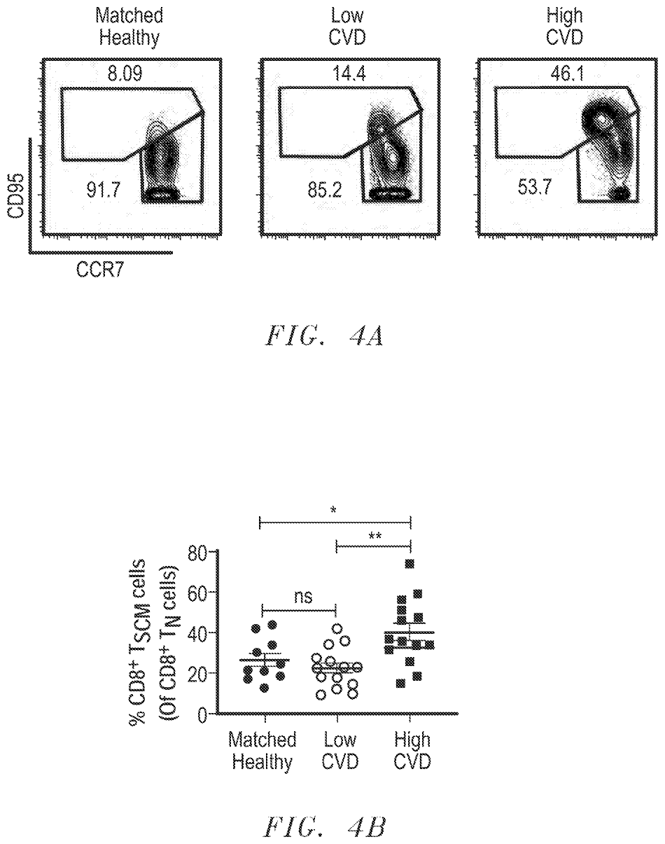

Identification of a subset of CD95+CD8+ T cells in subjects with high CVD. The inventors confirmed that individuals with high CVD displayed attenuated naive CD8+ T cell (CD8+ T_{N}) frequencies using manual flow gating via CD45RA and CCR7 expression (FIG. 3A). The inventors found significant, approximately 3-fold reductions in CD8+ T_{N} cells in high compared to low CVD individuals, shown here both as frequencies of CD8+ T cells (FIG. 3B) and of CD45+ leukocytes (FIG. 3C). In investigating the defining features of the naive CD8+ T cell compartment, the inventors discovered that individuals with high CVD had CD8+ T_{N} cells that expressed the memory antigen CD95 (FIG. 3D). Specifically, CD8+ T_{N} cells from subjects with high CVD displayed 3-fold elevations in CD95 frequencies (FIG. 3E), in addition to significant, 2-fold increases in CD95 geometric mean fluorescence intensity (gMFI) compared to CD8+ T_{N} cells from subjects with low CVD (FIG. 3F).

The memory T cell compartment is composed of heterogeneous T cell populations and conventionally divided into central memory, effector memory, and CD45RA+ effector memory CD8+ T cells with diverse functions. The recently described TSCM subset possesses naive-like properties (CD45RA+CCR7+CD27+CD28+) and the memory antigen CD95. Regarded as the least-differentiated memory population, TSCM cells are highly proliferative, capable of rapidly assuming effector roles, self-renewing, and multipotent. TSCM cells have been shown to possess potent anti-tumorigenic responses and are also increased in the periphery of subjects with Type 1 Diabetes (T1D), uveitis, and systemic lupus erythematosus (SLE).

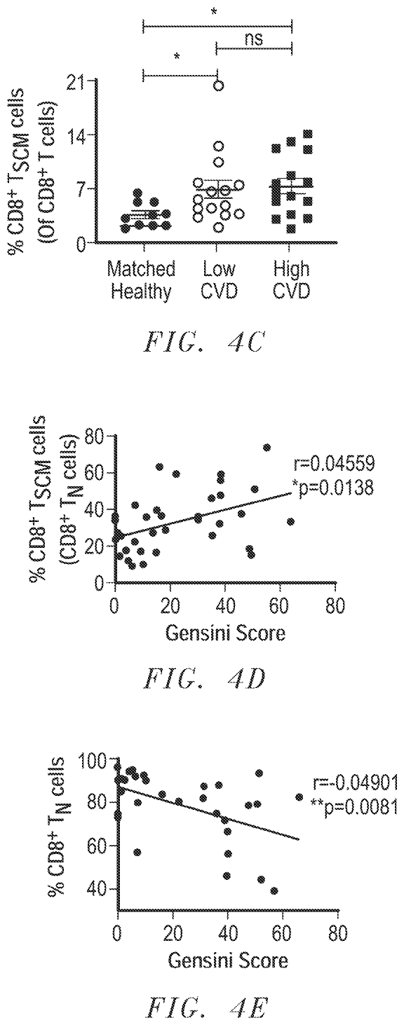

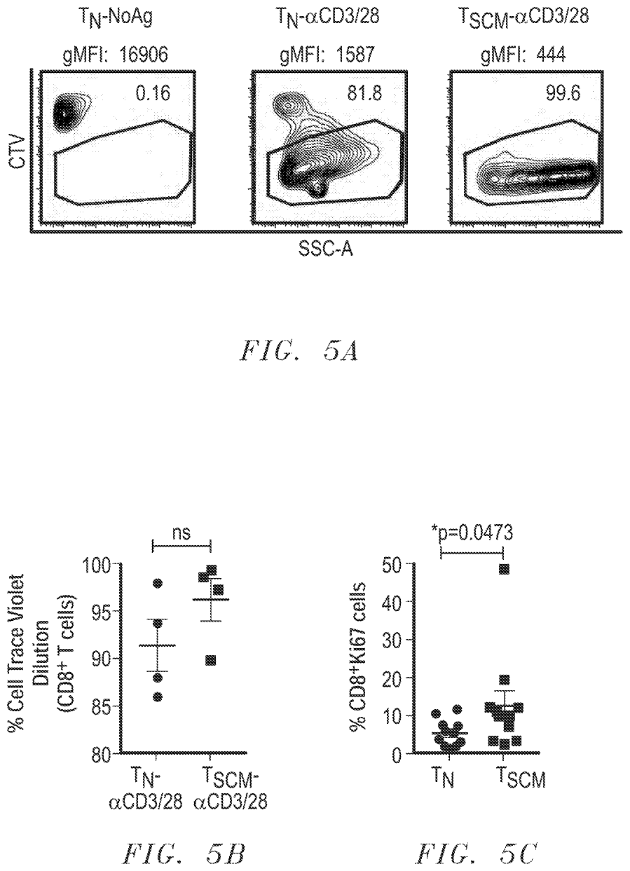

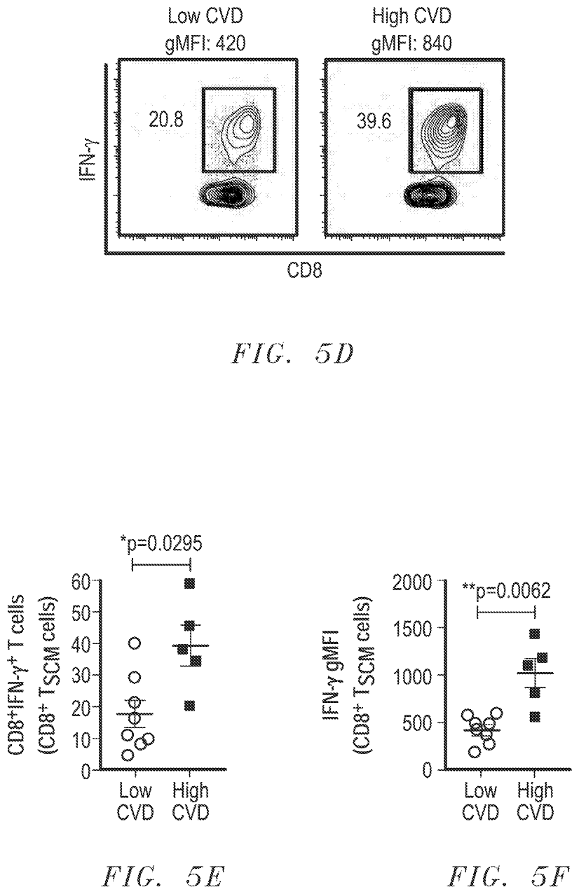

To determine whether CD8+ TSCM cells were elevated in subjects with high CVD, the inventors analyzed the mass cytometry data for the presence of TSCM cells using a previously-described gating strategy for human CD8+ T_{SCM} cells (CD45RA+CCR7+CD27+CD127+CD45ROloCD95+). The inventors found an approximate 2-to-3-fold increase in CD8+ TSCM frequencies using these markers in high compared to low CVD individuals (FIG. 4A). The inventors also examined TSCM frequencies within age-matched healthy controls. Interestingly, there was no statistically significant difference in CD8+ TSCM frequencies between low CVD individuals and age-matched healthy controls (FIG. 4A); however, there was a significant, 1.5-fold enhancement in CD8+ TSCM frequencies out of CD8+ TN cells, in high compared to low CVD individuals (FIG. 4B).

When the inventors assayed CD8+ TSCM frequencies within bulk CD8+ T cells, there was no statistical difference between low and high CVD subjects; however, age-matched healthy controls exhibited much lower CD8+ TSCM frequencies compared to individuals with either low or high CVD (FIG. 4C). Corroborating the increase in CD95 by CD8+ TN cells, CD8+ T_{SCM} frequencies positively correlated with clinical Gensini score (FIG. 4D), whereas CD8+ T_{N} frequencies were inversely correlated (FIG. 4E). These results indicate that the memory antigen CD95 on CD8+ T_{N} cells may serve as a biomarker of CVD severity and that CD8+ TSCM frequencies are enriched within the CD8+ T cell compartment with CVD compared to healthy controls.

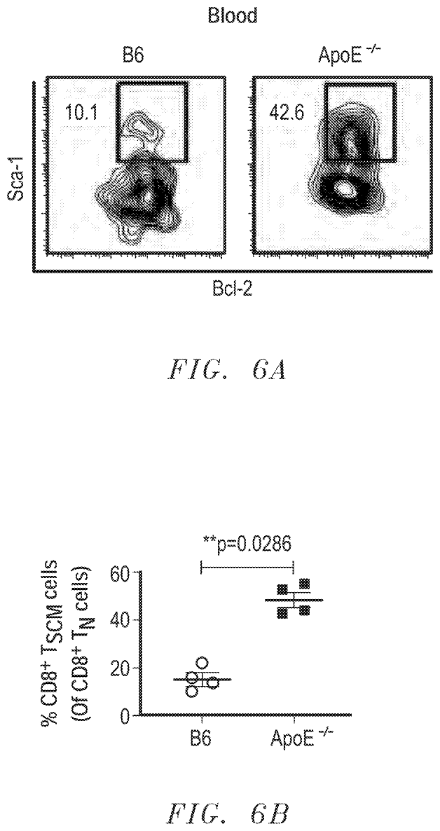

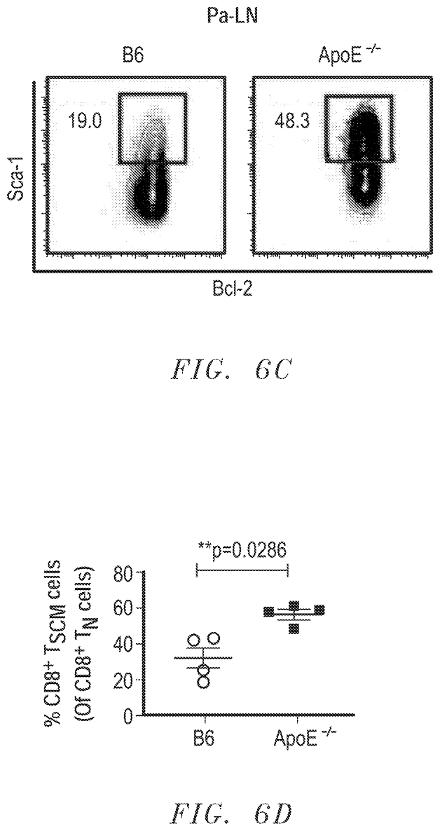

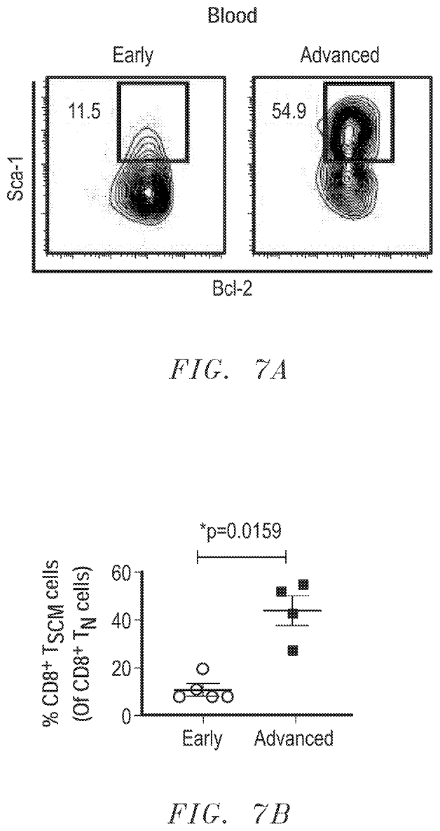

CD8+ TSCM cells are more proliferative than CD8+ TN cells and TSCM cells from high CVD subjects secrete increased IFN-γ, a pro-atherogenic cytokine. Numerous works have demonstrated that TSCM cells display increased proliferative capacity compared to TN cells. To examine CD8+ TSCM proliferation within CVD individuals, CD8+ TN and TSCM cells were sorted from low and high CVD individuals, stained with cell trace violet (CTV), and proliferative capacity was measured (FIG. 5A). TSCM cells displayed a slight increase in dilution of Cell Trace Violet (CTV), indicative of enhanced proliferation, compared to TN cells, although this did not quite reach statistical significance in this cohort (FIG. 5B). Interestingly, CD8+ TSCM cells were inherently more proliferative, with a 2-fold higher frequency of Ki67⁺ cells found within the CD8+ TSCM compartment compared to the CD8+ TN compartment. Next, the inventors wanted to determine if individuals with high CVD secreted more IFN-γ, a pro-atherogenic cytokine. CD8+ TSCM cells from high CVD individuals secreted 2.5-fold more IFN-γ than CD8+ TSCM cells from low CVD individuals, with an accompanying 2.4-fold increase observed in IFN-γ gMFI.

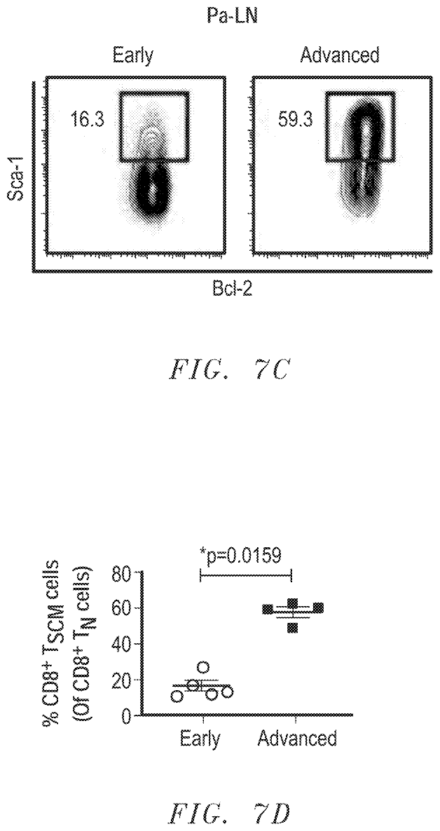

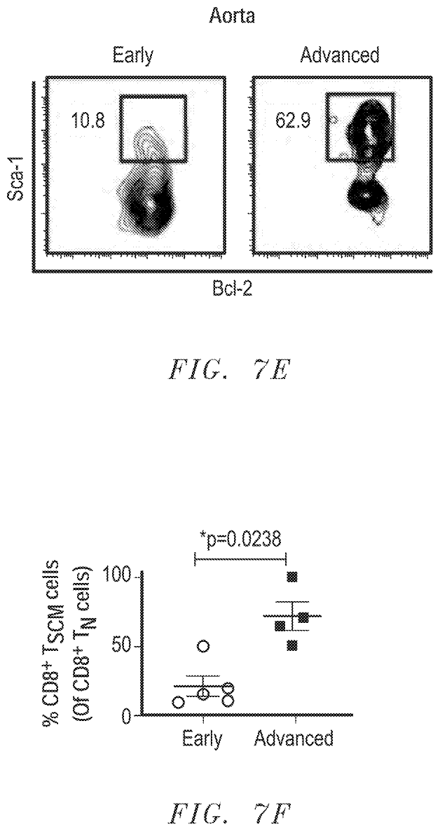

CD8+ TSCM cell frequencies are enhanced in atherosclerotic ApoE−/− mice. TSCM cells were originally identified in mice, defined as CD44lowCD62Lhigh TN cells, expressing stem cell antigen-1 (Sca-1). As there is no human ortholog for Sca-1, TSCM cells are phenotyped slightly differently in humans and mice, but within both species, there is noted expression of B cell lymphoma-2 (Bcl-2) and CXCR3 by TN cells. To determine whether CD8+ TSCM frequencies were increased in mice with atherosclerosis, CD8+ TSCM frequencies were examined in blood and para-aortic lymph nodes of ApoE−/− and C57BL/6 mice at 60 weeks of age. ApoE−/− mice displayed 10-fold increased CD8+ TSCM frequencies within the peripheral blood (FIG. 6A, B) and 4-fold increased CD8+ TSCM frequencies within the aorta-draining para-aortic lymph nodes (pa-LNs) (FIG. 6C, D).

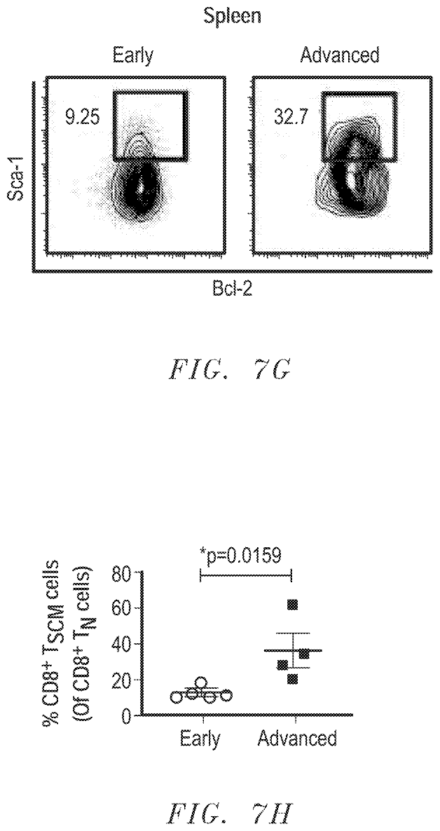

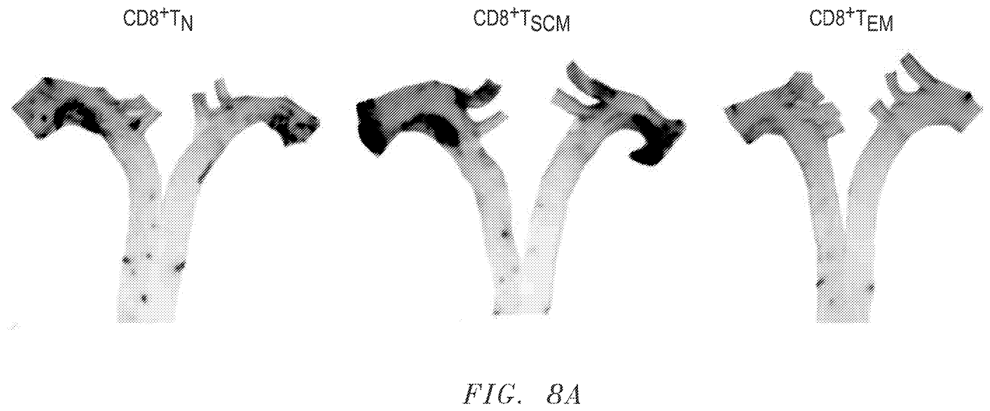

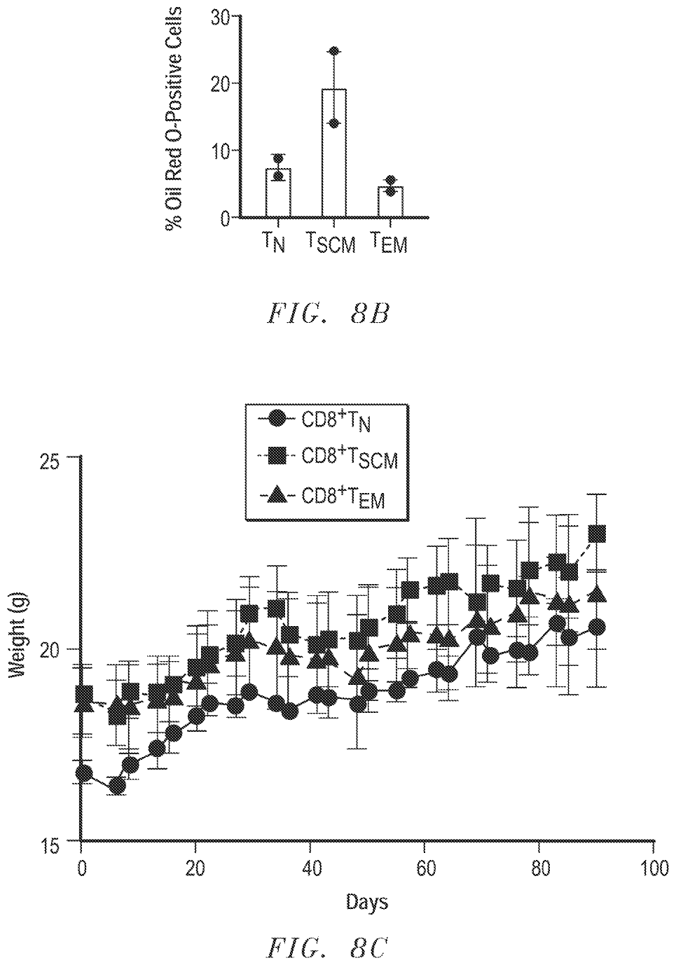

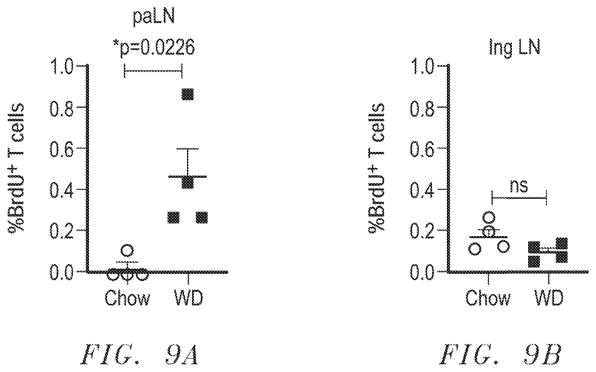

CD8+ TSCM cell frequencies are increased with advanced compared to early stages of atherosclerosis. To determine whether TSCM frequencies were enhanced in advanced stages of atherosclerosis, the inventors investigated these CD8+ TN cells expressing CD95 in early (mice fed chow at 60 weeks of age) compared to early stages of disease (11 weeks of age). Examining blood and aorta-draining pa-LNs revealed 6- and 3.6-fold increases in CD8+ TSCM cell frequencies, respectively (FIG. 7A, B, C, D). Excitingly, TSCM frequencies were 3.4-fold-elevated in advanced stages of atherosclerosis compared to B6 (FIG. 7E, F) and 3-fold increased in the spleen (FIG. 7G, H), recapitulating the results observed in high compared to low CVD individuals.

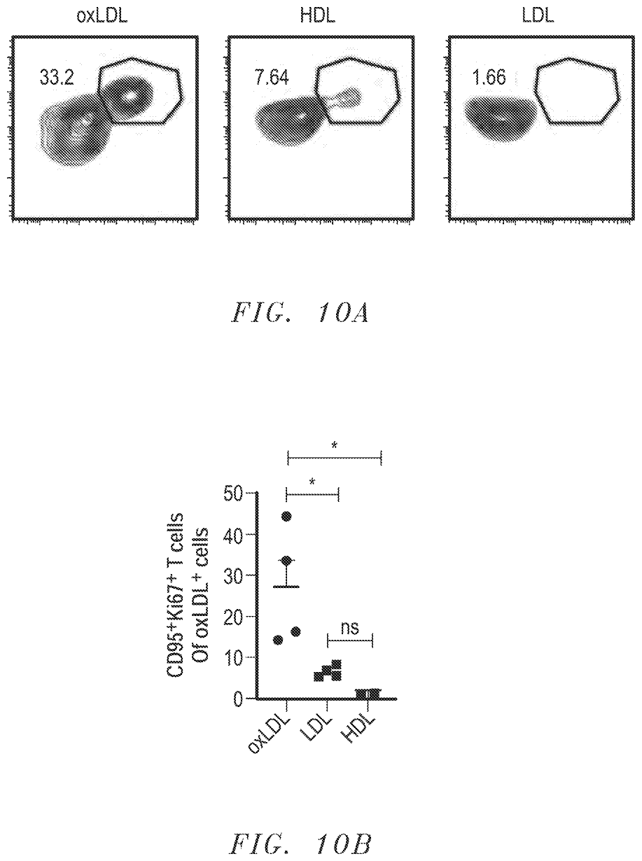

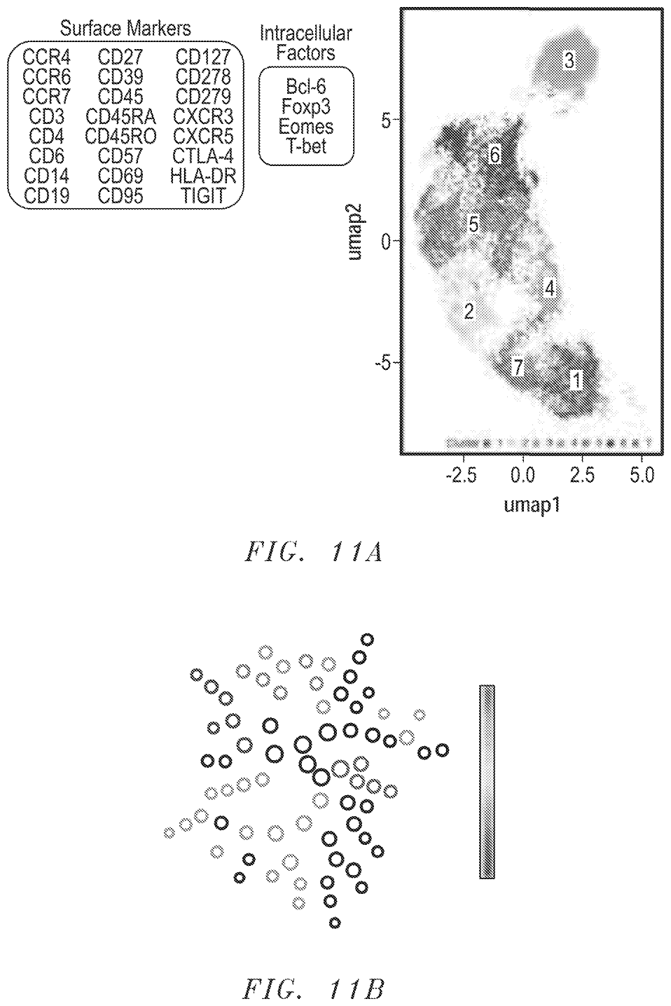

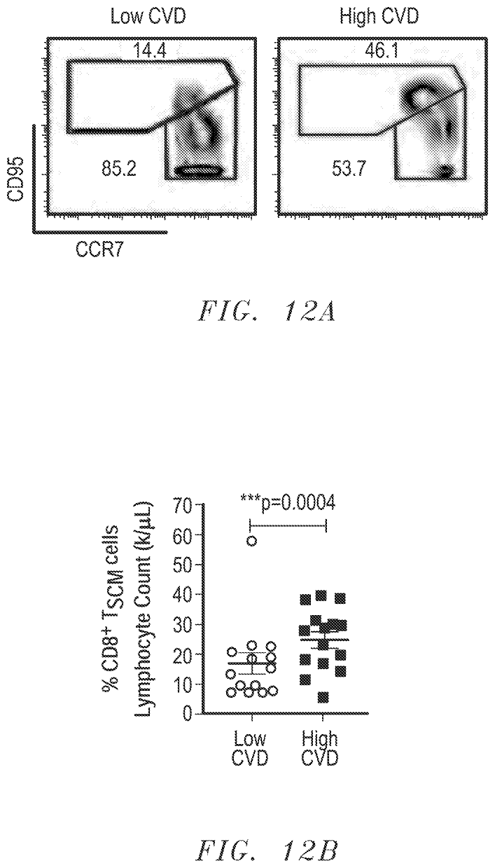

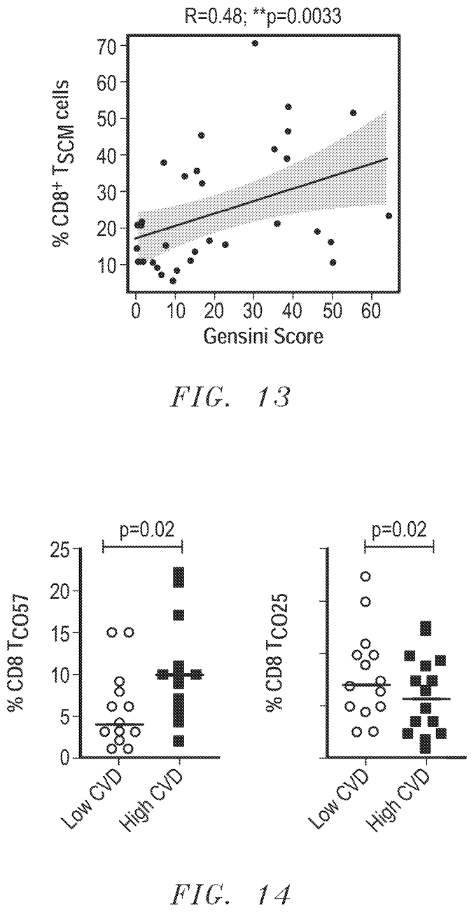

CD8+ TSCM adoptive transfer elicits increased atherosclerosis. To determine the role of CD8+ TSCM cells in atherogenesis, CD8+ TN, CD8+ TSCM, or CD8+ effector memory (TEM) cells were transferred into immune-deficient Rag.Ldlr recipients, and recipient mice were fed a high cholesterol diet for 12 weeks. Donor CD8+ T cell populations were co-transferred with purified CD4+T regulatory cells (Tregs) to slow cell division (AICD). As these studies are technically challenging and require a large starting bulk population, sorted CD8+ TN (CD44lowCD62LhighSca-llow), TSCM (CD44lowCD62LhighSca-lhigh), and effector memory (TEM) (CD44highCD62Llow) cells were injected into 2 recipient mice per group. In contrast to TN and TSCM cells, TEM cells preferentially traffic to peripheral tissues, where they assume rapid effector functions. Excitingly, analysis of the aortic roots at 12 weeks post high cholesterol diet revealed markedly increased Oil Red O+ cells, indicative of enhanced atherosclerosis disease severity in CD8+ TSCM adoptive transfer recipients in comparison to CD8+ TN or TEM recipients (FIG. 8A, B). This increase in atherosclerotic plaque formation was accompanied by no robust changes in weight loss within the TN, TSCM, or TEM transfer recipients (C). These studies indicate that CD8+ TSCM cells are pro-atherogenic.

Human CVD Subjects. Frozen peripheral blood mononuclear cells (PBMCs) from 28 human subjects were obtained from the Coronary Assessment in Virginia (CAVA) cohort, which includes patients from 30-80 years of age undergoing a medically necessary cardiac catheterization, with the following exclusion criteria: current acute coronary syndrome, cancer, autoimmune disease, anemia, pregnancy, HIV, immunosuppressive therapy, and prior organ transplantation⁴². Donor characteristics are outlined in (Table I). Medically necessary quantitative coronary angiography was performed, and percent aortic stenosis was calculated by two independent blinded investigators using Artis Workplace software (Siemens Medical Solutions). The Gensini Severity Scoring system was used to estimate CVD burden. The inventors analyzed PBMCs from 14 individuals with low CVD (Gensini score 0-10) and high CVD (Gensini score 30-60), in addition to 10 individuals with moderate CVD (Gensini score 10-30) (Table I). Protocols and procedures were approved by the Institutional Review Board at the University of Virginia (IRB HSR #15328), and blood was obtained after written informed consent.

Matched Healthy Controls. Heparinized blood was obtained from 10 self-reported healthy controls after written informed consent under the Institutional Review Board of the La Jolla Institute for Immunology (LJI) and in accordance with Dept of Health and Human Services Policy for the Protection of Human Research Subjects (IRB-VD-057-0217) Donor characteristics are detailed in (Table II). As individuals only provided year of birth, exact age is approximated. Peripheral blood mononuclear cells were isolated using Ficoll-Paque™ PLUS (GE Healthcare Biosciences AB) and SepMate-50 (StemCell Technologies), according to the manufacturer's protocol, resuspended in freezing media (10% DMSO/90% FBS) and stored in liquid nitrogen.

Mice. ApoE^{-/-} (B6.129P2-Apoe^{tm1Unc}/J) mice (Stock number: 002052) and C57BL/6 mice (Stock number: 000664) were purchased from The Jackson Laboratories (Bar Harbor, Me.). Rag.Ldlr mice were bred by crossing B6.129S7-Rag1^{tmlMom}/J (Rag^{-/-})×ApoE^{-/-} and maintained in the LJI animal facility. Male ApoE^{-/-} mice at 11 and 60 weeks of age were utilized for all assays. Rag.Ldlr recipients at 10-12 weeks of age were fed a high cholesterol diet for 12 weeks following adoptive transfer (1.25% cholesterol and 40% calories from fat) (Research Diets, D12108C). All mice were housed in microisolator cages in the pathogen free animal facility at LJI. All experiments followed the LJI Animal Care and Use Committee guidelines, and all studies were approved according to criteria outlined in the Guide for the Care and Use of Laboratory Animals from the National Institutes of Health.

Mass cytometry. Directly conjugated antibodies were purchased from Fluidigm and purified antibodies were ordered from the companies listed in the online-only Data Supplement. Conjugations were performed with the Maxpar X8 Multi-Metal Labeling Kit (Fluidigm) according to the manufacturer's instructions. Samples were stained for CyTOF mass cytometry, as described previously^{43,44} and stained with the antibody cocktail (Table III). Prior to surface staining, one low CVD sample, one high CVD sample, in addition to a staining healthy control were barcoded with CD45 (low CVD-CD45-89Y; High CVD-CD45-147Sm; Healthy-CD45-115Ln) (Table III). Cells were resuspended at 2×10⁶ cells/mL in 0.1 mL×EQ™ Four Calibration Beads (Fluidigm). Batch effects and signal drifting were minimized by tuning every 6 hrs during long runs. Data was normalized using the Matlab-based NormalizerR2013a_Win64.

Mass cytometry data analysis. Prior to downstream analysis, live CD45⁺CD3⁺ cells were manually gated in FlowJo v10.3.1. Resulting FCS were analyzed using R (v3.5.0) and Bioconductor (v3.7) packages. Protein expression was normalized using arcsin h transformation (cofactor=5) using CyTOF workflow⁴⁵ with pooled T cells from 28 low and high CVD individuals. Clustering was performed based on a self-organizing map (SOM) method via FlowSOM (v1.4.0) with default parameters.⁴⁶ Relative change in area under CDF (Cumulative Distribution Function) curve (ConsensusclusteringPlus v1.43.0)⁴⁷ was employed to determine cluster robustness with the number of clusters from 2 to 30. The inventors chose 15 clusters in which there was no appreciable increase. CyTOF data was mapped to two dimensions using a nonlinear dimensionality-reduction technique, uniform manifold approximation and projection (UMAP²⁰) which showed greater global cell structures compared to conventional tSNE. Differential test for the cluster abundance was performed using moderated test (originally from edgeR test for RNA-Seq data with modification for CyTOF from diffcyt package^{20,48}) and p-values were FDR-corrected for multiple testing. Data was visualized using ggplot (v2.2.2).

Human T_{SCM} Proliferation Studies. CD8⁺ T_{N} (CD45RA⁺CCR7⁺CD95^{lo}) and T_{SCM} cells (CD45RA⁺CCR7⁺CD95^{hi}) were sorted from high and low CVD individual PBMCs and stained with Cell Trace Violet (CTV, ThermoFisher) according to the manufacturer's guidelines. Cells were subsequently incubated with or without anti-CD3/CD28 Dynabeads (Gibco) in the presence of 30 U/mL recombinant human IL-2 (Peprotech) for 72 hrs at 37° C. with 5% CO₂. Sorted cells were cultured in RPMI with 10% FCS, L-glutamine, penicillin, and streptomycin, as described previously.

T_{SCM} Detection in peripheral blood, para-aortic lymph nodes, aorta, and spleen. Blood was collected by cardiac puncture, as described previously. Para-aortic lymph nodes, aorta, and spleen were collected aseptically. Spleens and lymph nodes were homogenized, and spleens and blood were RBC lysed using 1×RBC Lysis Buffer (Biolegend; San Diego, Calif.) and filtered through 40 μM cell strainers. Aortas were explanted and perfused with ice-cold PBS, minced, digested with DNAse I, Collagenase Type XI, and Hyaluronidase type I, and filtered through a 40 μM strainer, as described previously^{49,50}. Aortic cell suspensions were incubated at 37° C. for at least 30 minutes in RPMI with 10% FCS to restore expression of surface markers.

Atherosclerosis Severity Scoring. Mouse aortae were perfused with PBS, collected, and immersed in paraformaldehyde and stained with Oil Red O, then opened longitudinally, and pinned as described previously. Images were scanned, and the percentage of lesion surface area was quantified with Photoshop software.

CD8⁺ T_{SCM} Adoptive transfer. Spleens and LNs (inguinal, para-aortic, axillary, brachial, and cervical) were isolated aseptically from female B6 mice, and single cell suspensions were generated. CD8⁺ T_{N} (CD8⁺ TCRβ⁺CD44^{low}CD62L^{high}Sca-1^{low}), T_{SCM} (CD8⁺ TCRβ⁺CD44^{low}CD62L^{high}Sca-1^{high}) T_{EM} cells (CD8⁺ TCRβ⁺CD44^{high}CD62L^{Ow}) and Treg (CD4⁺CD25⁺GITR⁺) cells were enriched using a CD3 T cell isolation kit (StemCell), according to manufacturer's instructions. T cell-enriched populations were subsequently sorted utilizing an Aria-II. CD8⁺ T_{N}, CD8⁺ T_{SCM} cells, or CD8⁺ T_{EM} cells (2×10⁵) were adoptively transferred via intravenous injection into immune-deficient Rag.Ldlr recipients, with 7×10⁴ Treg cells, as described previously³⁴. All Rag.Ldlr recipients were bled prior to adoptive transfer to ensure immune deficiency. Mice were placed on a Clinton/Cybulsky high fat rodent diet with regular casein and 1.25% added cholesterol (Research Diets, Inc.; D12108C) for 12 weeks. Mice were weighed prior to injection and twice weekly following injection.

Flow Cytometry. For detection of human CD8+ T_{N} and T_{SCM} cells, low and high CVD PBMCs, in addition to age-matched healthy controls were thawed quickly at 37° C. and stained with CCR7 (G034H7) at 37° C. for 20 minutes, as described previously. Cells were subsequently surface stained with the following surface antibodies (Biolegend, CA except when indicated): CD4 (RPA-T4), CD8 (RPA-T8), CD27 (M-T271), CD95 (DX2), CD127 (A109D5), CD45RA (HI100), and CD45RO (UCHL1) for 30′ at 4 C. For proliferation studies, sorted CTV-stained cells were collected, and dynabeads were removed by magnetic selection, according to the manufacturer's instructions (Gibco). For IFN-γ detection, bulk PBMCs were stimulated for 72 hrs with dynabeads (Gibco). Cells were subsequently stained with CD4 (RPA-T4), CD8 (RPA-T8), CD27 (M-T271), CD45RA (HI100), CD45RO (UCHL1), and IFN-γ (B27). Murine blood, lymph nodes, spleen, and aortic cell suspensions were surface stained with the following antibodies (Biolegend; San Diego, Calif.): CD4 (GK1.5), CD8 (53-5.8), CD44 (IM7), CD45 (30-F11), CD62L (MEL-14), Sca-1 (D7), TCRβ (H57-597) and a fixable live-dead dye (Life Technologies; Carlsbad, Calif.). Cells were fixed with Fix/Perm buffer and permeabilized with Perm/Wash buffer, according to manufacturer's instructions (ThermoFisher). Cells were subsequently stained with the intracellular antibody Bcl-2 (BCL-10C4). Samples were acquired on an LSR-II (BD; San Diego, Calif.), and data were analyzed using FlowJo v10.3.1 (TreeStar; Ashland, Oreg.).

Statistical Analyses. All results are expressed as mean+s.e.m. Results were analyzed by unpaired t-test. Linear regression and pearson's correlation coefficient (r) were utilized to were used to determine the statistical significance and correlation in the human studies. Sample size was based on previous studies, consulting with biostatisticians, and using nQuery Advisor 6.0 software. For in vitro studies, statistical method was used to determine sample size. The data seemed to be normally distributed with similar s.d. and error observations between experimental groups and controls. A p value of <0.05 is considered to be significant. Statistical analysis was performed using GraphPad Prism software version 6 (GraphPad Software, Inc.).

### Example 2. CD8 T Lymphocyte Subsets in Human Atherosclerosis

Cardiovascular disease (CVD) kills one in four people annually in the United States and remains a major health concern, despite the development of statins and other lipid-lowering drugs. Atherosclerosis is the main underlying trigger of CVD and a chronic immune disease. T lymphocytes accumulate within vascular plaques during atherosclerosis progression. CD4 T cells are well-characterized in atherosclerosis, but the importance of CD8 T cells in the disease is far less understood. The inventors performed high-dimensional mass cytometry of peripheral blood T cells from human subjects with low- and high-risk CVD. A profound loss of circulating naïve CD8 T cells (T_{N}) was found in subjects with high-versus low-risk CVD. Further, subjects with high-risk CVD showed a significant increase in a CD8 T stem cell memory (T_{SCM}) cell population that expresses the memory antigen CD95, and also an increase in a senescent CD8 T cell subset that expresses CD57 (T_{CD57}). The inventors also identified the loss of a CD8 T cell that expresses CD25 (T_{CD25}). As shown in Example 1, the T_{SCM} population was proatherogenic in mice. Thus, CD8 T_{SCM} cells are proatherogenic and predictive of high CVD-risk in humans. It is further found that the T_{CD57} subset is unable to mount an effective immune response in subject with high CVD risk. The T_{CD25} subset may be a regulatory subset that is atheroprotective, hence its reduction in subjects with high CVD risk. These 3 newly identified human CD8 T cell subsets can be linked to atherosclerosis and CVD events using the Multi-Ethnic Study of Atherosclerosis (MESA) cohort. MESA is an NHLBI, longitudinal cohort established in 2000-2002 with 6000+ subjects, all of which were 45-85 years old and CVD-free upon matriculation into the study. Besides its size, another strength of MESA is its inclusion of both males and females from 4 ethnic groups (Caucasian, African-American, Hispanic, and Asian). It is also possible to test how these newly identified CD8 T cells directly impact atherosclerosis. The mechanistic contributions of these new CD8 T cells to atheroprogression can now be determined via adoptive transfer of these cells into established, atherosclerosis-prone mouse strains. Thus, the present invention can be used to identify mechanisms by which CD8 T cell subsets orchestrate human atherosclerosis that can also be used to identify an test new therapies for CVD.

CD8 T Lymphocyte Subsets in Human Atherosclerosis. Despite the development of statins and other treatments, cardiovascular disease (CVD) remains a leading cause of death worldwide. T lymphocytes play an important role in regulating atherosclerotic disease progression and are divided into two main classes: CD4 and CD8. CD4 T lymphocytes are key regulators of CVD, with evidence indicating that CD4 T helper 1 (Tₕ1) cells promote atherosclerosis and CD4 T regulatory (T_{Reg}) cells protect against the disease_{1,2}. Much less is known about CD8 T cells in CVD₃, with the contributions of specific CD8 T cell subsets to atherosclerosis still uncharacterized. Most studies on CD8 T cells in CVD have been performed in mice and suggest contradictory models. For example, antibody-based depletion of CD8 T cells in mice is atheroprotective, whereas genetic ablation of CD8 T cells in mice does not affect atherosclerosis_{4,5}. These results may be explained by the observation that there are lower numbers of CD8 T cells present in the atherosclerotic plaques of mice compared to humans, as well as differences in the timing of these studies. CD8 T cells are enriched in human atherosclerotic plaques and hyperactivated compared to CD4 T cells_{3,6-8}, but their roles in disease progression are not well studied and the CD8 subsets that emerge in human CVD are undefined.

To identify CD8 T cells that impact human CVD, the inventors performed mass cytometry on blood from patients undergoing medically necessary coronary angiography as part of the Coronary Assessment in Virginia (CAVA) cohort at the University of Virginia₉. They correlated these mass cytometry results with each patient's coronary angiography-derived Gensini score, which accurately measures the extent of clinical atherosclerosis₁₀. The inventors found a reduction in naïve (CD45RA⁺CCR7⁺CD27⁺CD28⁺) CD8 T (T_{N}) cells in “high CVD” subjects (patients who had Gensini scores>20). High CVD patients also exhibited increased frequencies of a distinct CD8 cell type expressing T_{N} cell markers plus the memory antigen CD95, termed stem cell memory T (T_{SCM}) cells. These CD8 T_{SCM} cells have never been reported in cardiovascular disease. Example 1 includes the data from the CAVA cohort showing that T_{SCM} cells are positively correlated with Gensini scores, suggesting that these cells participate in atheroprogression. T_{SCM} cells are the least differentiated memory CD8 T cell subset, highly proliferative, and can display effector T cell phenotypes. Example 1 shows that CD8 T_{SCM} cells are predictive of high CVD risk. The inventors also observed an increase in a senescent CD8⁺CD57⁺ (CD8 TCD57) subset¹² and the loss of a CD25⁺ (CD8 T_{CD25}) subset, in subjects with high risk CVD. CD8 TCD57 cells are senescent or exhausted, and pro-atherogenic, whereas CD8 T_{CD25} cells are an atheroprotective T_{Reg} subset.

Analyze the expression, functions, and clinical associations of newly identified CD8 T cell subsets in human atherosclerosis. Using the present invention it is possible to study subjects from the NHLBI longitudinal Multi-Ethnic Study of Atherosclerosis (MESA) cohort. MESA was established in 2000-2002 (Exam 1) with 6,814 subjects, 45-85 years old and free of clinical CVD. MESA participants have been followed through 2016-2018 (Exam 6) for numerous biomarkers of subclinical atherosclerosis and events. As a MESA participant, the present inventors used peripheral blood mononuclear cell (PBMC) samples and matched clinical data, including adjudicated myocardial infarction (MI) events and detailed blood chemistry and lipid profiles, from 900 patients in this cohort. Within MESA, CVD risk is determined by coronary artery calcium (CAC) Agatson scoring. CAC scores are correlated with increased CVD and MI risk_{13,14}. Cryopreserved PBMC samples from Exam 1 subjects with low (=0 HU) and high (>300 HU) CAC scores will determine whether peripheral CD8 T_{SCM}, CD8 T_{CD57}, and CD8 T_{CD25} cells are differentially increased in high CVD subjects (both sex- and ancestry-specific analyses are evaluate in the cohort). Using novel single-cell RNA-sequencing (scRNA-seq) methods, CD8 T_{SCM}, CD8 T_{CD57}, and CD8 T_{CD25} transcriptomes are compared between high- and low-risk CVD MESA subjects. To functionally interrogate how CD8 T cells differ in low-versus high-risk CVD patients, CD8 T cell proliferation, activation/exhaustion, and cytokine production are measured. Such phenotypes are tested for correlations with clinical data.

CD8 T_{SCM}, CD8 T_{CD57}, and CD8 T_{CD25} cells impact atherosclerosis. The inventors found that CD8 T_{SCM} cells promote atherosclerosis. Using the present invention it is possible to compared CD8 T_{SCM} cells to either central (CD8 T_{CM}) or effector memory T (CD8 T_{EM}) cells. It is also possible to transfer mouse CD8 T_{N}, CD8 T_{CD57}, CD8 T_{CD25}, CD8 T_{SCM}, CD8 T_{CM}, and CD8 T_{EM} cells into lymphocyte-deficient, Rag.Ldlr−/− recipient mice fed a high-cholesterol diet (HCD) and quantify atherosclerosis. If CD8 T_{SCM} cells preferentially differentiate into either T_{CM} or T_{EM} cells in mice fed an HCD diet can be determined. It is also possible to determine if oxidized low density lipoproteins (oxLDLs) influence the development or functions of these subsets.

Adaptive immunity in CVD. T cells are recruited into atherosclerotic plaques by inflammatory signaling. Extensive research has been carried out on the roles of CD4 T cells in atherosclerosis. In particular, numerous studies have demonstrated that CD4 Tₕ1 cells are pathogenic, while T_{Reg} cells protect against atherosclerosis-induced inflammation_{1,2,15,16}. CD8 T cells carry out both atheroprotective and atherogenic roles₃. Still, there is a paucity of research on the importance of specific CD8 T cell subsets in atherosclerosis and CVD progression, despite their prominence in human atherosclerotic lesions_{7,17}. It is now possible explore the importance of CD8 T cell populations in atherosclerosis and CVD progression.

CD8 T cells and CVD. The role of CD8 T cells in human CVD progression remains unclear. Studies have mostly demonstrated pro-atherogenic, atheroprotective, or dispensable roles for CD8 T cells in mice. For example, while depletion of CD8 T cells utilizing monoclonal antibodies reduces disease progression in atherosclerosis-prone, apolipoprotein E-deficient (Apoe_{-/-}) mice₅ and adoptive transfer of CD8 T cells heightens atherosclerosis pathology₅, depletion of the CD8a gene within Apoe_{-/-} mice has no impact₄. Moreover, immunization of Apoe_{-/-} mice with p210, a peptide that selectively expands CD8 T cells, reduces atherosclerotic lesions₁₈, supporting a possible atheroprotecitve role. MHC-I-deficient mice, which present with diminished CD8 T cells, exhibit increased plaque size when fed a high-fat diet₁₉. Additionally, adoptive transfer of CD8₊CD25₊ T cells into Apoe_{-/-} mice limits atheroprogression₂₀.

By way of explanation, and in no way a limitation of the present invention, in humans, evidence suggests that circulating CD8 T cells are associated with CVD progression. During early CVD, CD8 T cells constitute a third of all leukocytes within the vascular intima and increase to approximately 50% in advanced atherosclerotic plaques, with cytotoxic CD8 T cells constituting the bulk of this population_{3,6,7}. Increased circulating CD8₊CD57₊ T cells are observed in human CVD patients₂₁, and elevated CD8₊CD57₊ T cell frequencies are linked to short-term cardiovascular mortality in individuals with acute myocardial infarctions (MI)₂₂. Furthermore, circulating CD8 T cells correlate with incidences of coronary events₂₃.

T memory stem (T_{SCM}) cells. The memory T cell compartment comprises a heterogeneous population conventionally divided into T_{CM}, T_{EM}, and CD45RA effector memory (TEMRA) cells²⁴. As shown in Example 1, stem cell memory T cell (T_{SCM}) subset makes up 2-3% of the circulating T cell pool in healthy individuals and is present in mice²⁵, non-human primates²⁶, and humans²⁷. Regarded as the least-differentiated memory population, T_{SCM} cells express naïve T cell markers (CD45RA+CCR7+CD27+CD28+) and the memory marker CD95 and are highly proliferative, multipotent, and capable of rapidly assuming effector roles^{25, 27}. The factors responsible for T_{SCM} cell fate commitment have yet to be fully characterized. Still, the T_{SCM} transcriptional program likely shares important features with that of T_{CM} cells²⁸, including reliance on Wnt-β and interleukin (IL)-7 and IL-15 signaling^{25, 27, 29, 30}. T_{SCM} cells display potent anti-tumorigenic properties and are increased in the periphery of patients with inflammatory conditions such as Type 1 Diabetes₃₁, uveitis₃₂, and systemic lupus erythematosus (SLE)₃₃. As shown hereinabove in Example 1, CD8 T_{SCM} cells are pro-atherogenic.

Coronary artery calcium and CVD risk. Calcification of vascular plaques occurs during advanced stages of atherosclerosis and produces a feedback loop that yields increased local inflammation and plaque rupture₃₄₋₃₉. Plaque calcification can be estimated using imaging protocols that generate coronary artery calcium (CAC) Agaston scoring, with the presence of subintimal coronary calcification indicative of atherosclerosis₃₄. Indeed, as a non-invasive diagnostic tool, CAC Agatson scoring is routinely performed during computed tomography (CT) scans. CAC scores increase with atherosclerosis progression and high CAC Agatson scores (above 300 HU) are strong predictors of future CVD events_{40, 41}. Additionally, progression of CAC is significantly associated with increased mortality₄₂. Indeed, the St. Francis Heart Study evaluated the usefulness of CAC scoring in predicting cardiac events and found that subjects without an event exhibited median CAC scores of 4 Hounsfield units (HU) whereas subjects with an event displayed median scores>200 HU₄₃. This finding matches previous results from the Multi-Ethnic Study of Atherosclerosis₄₀.

Multi-Ethnic Study of Atherosclerosis (MESA). Established between 2000 and 2002 (Exam 1), MESA is an NHLBI longitudinal cohort study aimed at identifying features of subclinical atherosclerosis, as well as risk factors that predict clinically-detectable CVD_{44, 45}. The 6,814 MESA participants at Exam 1 consisted of males and females with 38% Caucasian, 28% African American, 22% Hispanic, and 12% Asian ancestry, between 45-84 years of age and free of clinical CVD at Exam 1 (recruitment)_{44, 45}. MESA subjects have participated in five follow-up visits for plasma collection and assessment of their clinical parameters, approximately every 3 years_{44, 45}. Participants excluded from MESA at Exam 1 included those with active CVD, a previous MI, active treatments for cancer, pregnancy, a need for a CT scan in the past year, or any serious medical condition that precluded long-term involvement_{44, 45}. MESA samples and outcome and event data can be accessed including risk factors and biomarkers from approximately 1000 MESA subjects.

CD8 T cell heterogeneity and functions in human atherosclerosis remain ill-defined. The current understanding of CD8 T cell subsets in atherosclerosis remains behind that of CD4 T cell involvement in atheroprogression. The inventors identified a significant elevation in CD8 T_{SCM} cells in human subjects with CVD. CD8 T_{SCM} cells are highly proliferative and produce pro-inflammatory cytokines. While this cell type has been studied in cancer_{27, 46-53}, the inventors are the first to report its association with human CVD (See Example 1). The inventors have also identified 2 human CD8, and one CD4, subsets whose numbers differ in blood of low versus high CVD subjects. Neither of these subsets has been studied functionally in human or mouse CVD. Studies on the numbers, heterogeneity, or functions of CD8 cells in human atherosclerosis progression and adverse CVD events have been fews_{54, 55} and limited in the 20 ongoing years of the MESA cohort.

Using the present invention, it is possible to mechanistically understand the functions of the new CD8 T cell subsets that have never been studied in human or mouse atherosclerosis. Blood samples from low- and high-risk CVD patients from the NHLBI-sponsored MESA clinical cohort can be analysed for CD8 T cell subset phenotypes that can be linked with several hundred clinical parameters. Secondly, state-of-the-art Antibody-seq (Ab-seq) and Cellular Indexing of Transcriptomes and Epitopes by Sequencing (CITE-seq) approaches can be used to simultaneously identify surface protein and gene expression signatures of CD8 T cells from MESA patients at the single-cell level. Adoptively transfer CD8 subsets are injected into atherosclerotic mice to determine how the CD8 T cell subsets mechanistically impact atherosclerosis.

Sex as a biological variable (SABV). Due to known sex-differences in atherosclerosis development_{56, 57}, both male and female samples are used for human and mouse experiments, thus identifying differences between males and females and any disparities are followed up with additional experimentation and analyses. MESA cohort participants without baseline statin use that are matched on sex, race, current- and ever-smoking status, and age within 5 years are studied. Using these restrictions, the inventors identified 192 matched pairs of low CVD (defined by CAC scores=0) and high CVD risk (defined by CAC scores>300). These consisted of 99 white pairs, 55 African American pairs, and 38 Hispanic pairs (see Table 4). Next, 10 males and 10 females with low CVD-risk (defined by CAC scores=0) and high CVD-risk (defined by CAC scores>300), matched as above, from each race/ethnic group provide a 90% power to detect significant (p<0.05) changes in T_{SCM}, CD8 TCD57, and CD8 TCD25 subsets between low and high CVD-risk patients via BD Rhapsody (see Table 4) are studied. Flow cytometry and functional analysis utilize an additional 5-15 subjects per group as per prior power calculations_{11, 58,59}. Primary analyses can be pooled across race/ethnic groups with covariate adjustment for potential confounders including diabetes status, systolic blood pressure and lipid levels. To account for multiple comparisons in the 3 different CD8 T cell subsets under consideration, a Bonferroni corrected threshold is used for statistical significance defined as a*=0.05/3 CD8 T cell subsets=0.016, while the nominal level of a=0.05 can be used as a suggestive threshold. Additional secondary analyses are stratified by race/ethnic groups and sex.

TABLE 4
Demographic breakdown of patient samples.

| | | | | Patient Cohort | | | |
|---|---|---|---|---|---|---|---|
| | | | | Aim 1a - | Aim 1a - | Aim 1b - | |
| | | | Number of | Discovery | Validation | Functional | |
| Ancestry | Sex | CAC Scores | Subjects | (BD Rhapsody) | (Flow Cytometry) | Studies | Totals |
| Caucasian | Male | High (>300) | 52 | 10 | 5 | 15 | 30 |
| | | Low (=0) | 52 | 10 | 5 | 15 | 30 |
| | Female | High (>300) | 47 | 10 | 5 | 15 | 30 |
| | | Low (=0) | 47 | 10 | 5 | 15 | 30 |
| African | Male | High (>300) | 30 | 10 | 5 | 15 | 30 |
| American | | Low (=0) | 30 | 10 | 5 | 15 | 30 |
| | Female | High (>300) | 25 | 10 | 5 | 10 | 25 |
| | | Low (=0) | 25 | 10 | 5 | 10 | 25 |
| Hispanic | Male | High (>300) | 25 | 10 | 5 | 10 | 25 |
| | | Low (=0) | 25 | 10 | 5 | 10 | 25 |
| | Female | High (>300) | 13 | 10 | 3 | — | 13 |
| | | Low (=0) | 13 | 10 | 3 | — | 13 |

| | | | | | |
|---|---|---|---|---|---|
| Totals | 384 | 120 | 56 | 130 | 306 |

Any statistically significant differences found in CD8 T cells within African American subjects are followed up with additional experimentation and analyses, using PBMC samples from the Jackson Heart Study (JHS), which includes African Americans from Jackson, Miss. metropolitan area. The JHS recruited over 5,000 individuals from 291 families to study genetic and environmental influences on cardiovascular disease in African Americans^{60, 61}.

For animal studies, nQuery Advisor 6.0 software is used, and considered various animal models with respect to effect size, type I error, and power. Through this analysis, the inventors find that atherosclerosis analysis and detection of CD8 T cell phenotypes by conventional flow cytometry with 90% power and 1% type I error is achievable with 15 mice per group (Control versus Experimental). Males and females can be studied separately, and all mice are on a congenic C57BL/6J background. To ensure that the allocation, treatment, and handling of mice is uniform, animals within each experimental group are randomized and data is subjected to blinded analyses. The recommendations of the American Heart Association's Scientific Statement on Recommendation on Design, Execution, and Reporting of Animal Atherosclerosis Studies are followed General Statistics: Mean responses amongst groups are compared and data reported as means (±SEM). Group or single-measurement differences are determined by an ANOVA or Student's t-test as necessary. All data is analyzed using Prism v8 (GraphPad).

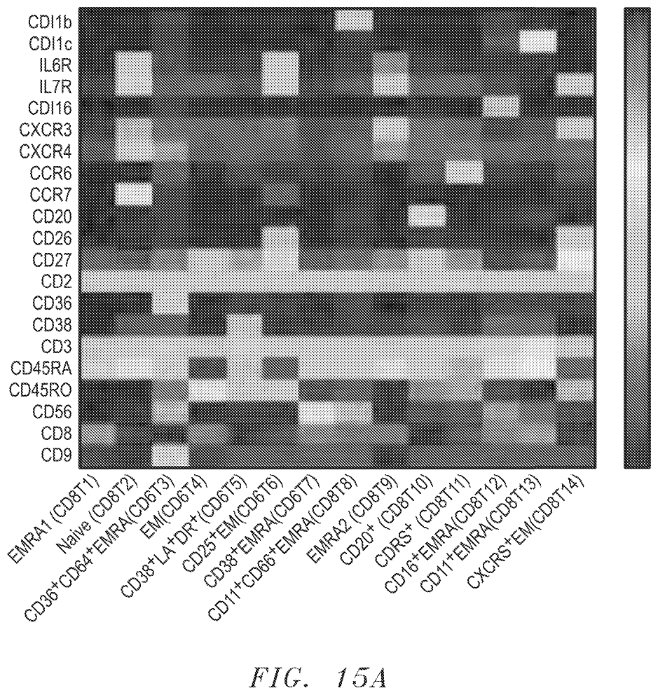

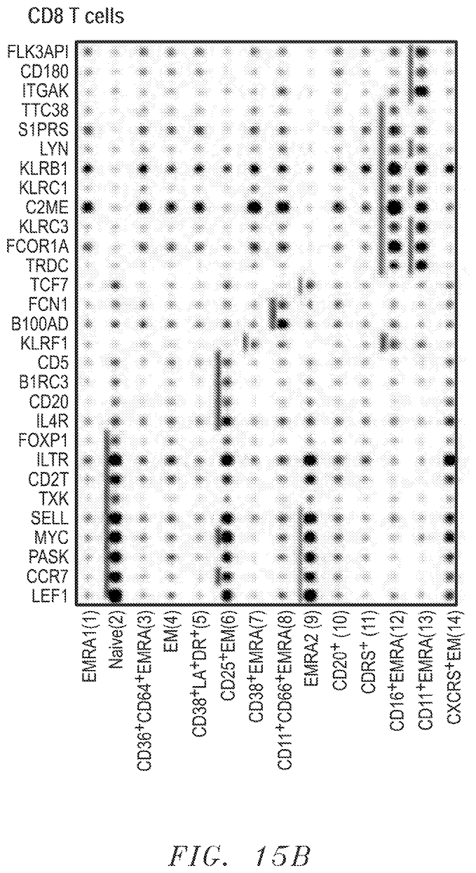

Analyze the expression, functions, and clinical associations of newly identified CD8 T cell subsets in human atherosclerosis. CD8 T cells constitute the largest proportion of T lymphocytes present in human atherosclerotic plaques, yet their functions in atheroprogression are not well understood. Using the present invention, it is possible to study how the frequencies and functions of CD8 T cells contribute to atherosclerotic burden and CVD risk in the MESA cohort. CD8 T cells from subjects undergoing medically-necessary angiography as part of the Coronary Assessment in Virginia (CAVA) cohort can be used₉. The CAVA cohort includes patients 30-80 years of age undergoing medically necessary cardiac catheterization. Exclusion criteria for CAVA include the presence of acute coronary syndrome, cancer, autoimmune disease, anemia, pregnancy, HIV, immunosuppressive therapy, and prior organ transplantation. All participants in CAVA undergo quantitative coronary angiography at UVA and the Gensini Severity Scoring system is used to accurately estimate their CVD burden₁₀. Patients were stratified on the basis of Gensini scores as either having low CVD (scores=0-6) and high CVD (score>20). Low CVD subjects were considered a control group since they have no stenosis. CD8 lymphocytes from CAVA subjects were analyzed by high-dimensional mass cytometry (CyTOF) and the cluster identification, characterization, and regression (CITRUS) algorithm (FIGS. 11A and 11B). Shown in FIG. 11A is the CyTOF panel. The UMAP in FIG. 11A, which plots the CyTOF data onto a 2-dimensional biaxial plot, shows 7 clusters or subsets of human CD8 T cells identified by the CyTOF panel taught herein in CAVA subjects. Each dot is a single cell and each color/shade is a phenotypically different human CD8 subset. The power of CITRUS is its ability to identify parameters that predict clinical outcomes for conditions including lupus, lung cancer, and other diseases₆₃₋₆₆. CITRUS identified key cell profiles that were different between low and high-risk CVD subjects within CAVA (red and green dots in FIG. 11B). CITRUS identified an approximately 2-fold reduction in the abundance of a CD8 T cell population (green cluster shown in UMAP in FIG. 11A). Manual gating of conventional flow cytometry data confirmed that this population represented the naïve CD8 T (T_{N}) cell (CD8₊CD45RA₊CCR7₊) strategy for CD8 T_{SCM} cells (CD45RA⁺CCR7⁺CD27⁺CD127⁺CD45RO^{low}CD95⁺) compartment (see Example 1), and further investigation of this population using CITRUS revealed upregulation of the memory antigen CD95 within these cells in high CVD-risk individuals. Specifically, CD8 T_{N} cells from subjects with high CVD displayed a 3-fold elevation in CD95₊ frequencies (FIG. 12A). The inventors analyzed the CyTOF data for the presence of T_{SCM} cells described in Example 1. Subsequently, the inventors found an almost 2-fold increase in CD8 T_{SCM} frequencies in high versus low CVD individuals. The inventors also found no statistically significant difference in CD8 T_{SCM} frequencies between low CVD individuals and age-matched healthy controls. (FIG. 12B). Importantly, the inventors found that CD8 T_{SCM} frequencies positively correlated with clinical Gensini scores (FIG. 13). These results indicate that CD8 T_{SCM} frequencies are enriched within the CD8 T cell compartment of high-risk CVD patients, and that expression of the memory antigen CD95 on CD8 T_{N} cells is strongly associated with clinical CVD severity. To expand on this pro-inflammatory notion whether CD8 T_{SCM} cells from high-risk CVD patients produce increased by CD95, gated on CD45RA CD27 CD127 CD45RO cells levels of the proinflammatory cytokine interferon (IFN)-γ was determined. CD8 T_{SCM} cells from individuals with high CVD-risk secreted 2.5-fold more IFN-γ than those from low-risk CVD subjects (see FIGS. 5D and 5E). These results show that T_{SCM} cells embody a pro-inflammatory CD8 T cell subset.

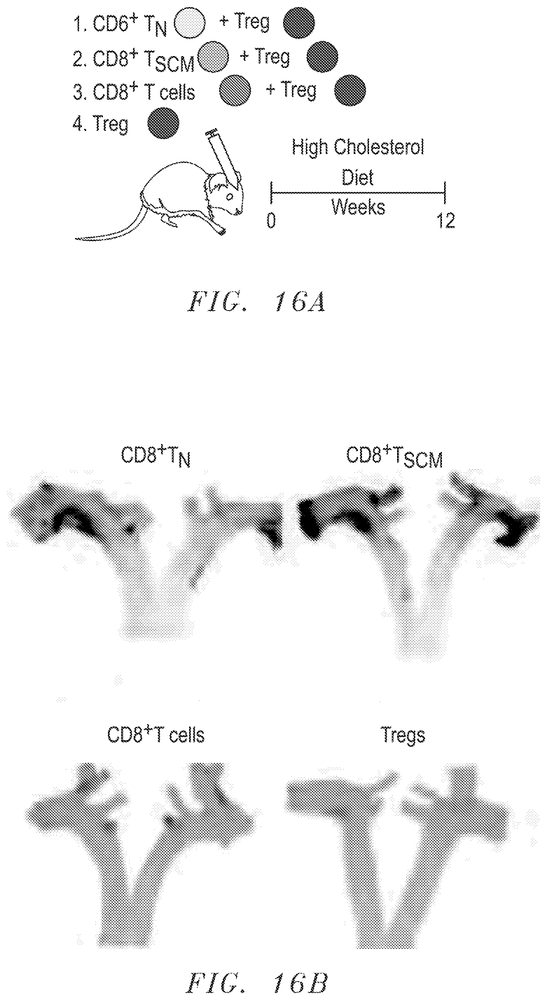

Reviewing existing CAVA cohort CyTOF data to expand the studies of CD8 T cells. The inventors identified two additional CD8 T cell subsets that are differentially abundant in high versus low CVD subjects in this same CAVA cohort (see FIGS. 11 and 14). Specifically, the inventors found a significant increase in a CD8₊t-bet⁺CD57⁺ subset (termed as CD8 T_{CD57}; purple subset in FIG. 11 and left panel in FIG. 14) and a slight, almost-significant decrease in a potentially novel CD8⁺CD25⁺ T_{Reg} subset (termed as CD8 T_{CD25}; yellow subset in FIG. 1 and right panel in FIG. 14). This T_{CD25} subset may be immune suppressive and atheroprotective, like their CD4+CD25+CD127loFoxp3+ counterparts. CD57 has been identified on a subset of CD8+CD45RA+ cytotoxic T cells that are considered to be senescent or exhausted67. Other studies suggest that CD8+CD57+ cells may not be exhausted, but rather are highly differentiated and capable of rapid activation and IFNγ production¹². Neither of these identified CD8 cellular subpopulations have been studied in the context of atherosclerosis. Thus, heterogeneity within the CD8 T cell compartment can be determined, with a focus on CD8 T_{SCM}, CD8 T_{CD57}, and CD8 TCD25 newly identified subsets, to visualize phenotypic and functional changes in CD8 T_{SCM} cells or other CD8 populations between subjects with low and high CVD-risk, and, to link CD8 subset function with clinical parameters indicative of CVD risk.

The heterogeneity of CD8 T cells in the MESA cohort can be examined, in which CVD risk is accurately quantified by CAC Agatson scoring. CAC scores increase with atherosclerosis progression and high CAC Agatson scores (above 300 HU) are strong predictors of future CVD events_{40, 41}. As MESA has collected data on a diverse array of clinical data, it is also possible to correlate the frequencies of CD8 T_{N}, CD8 T_{SCM}, CD8 T_{CD57} and CD8 T_{CD25} cells to a range of clinical parameters, such as MI incidence, lipid profiles, immune cell counts, and plasma oxLDL content, to understand the link between CD8 subsets and CVD risk in humans. Such parameters have been studied extensively for CD4 T cells, but not for CD8 subsets. Using these data it is also possible to determine the transcriptional and functional profiles of CD8 T_{SCM} and other CD8 T cell subsets in low and high CVD-risk individuals within the MESA cohort through use of the BD Rhapsody platform, which measures both protein and gene expression simultaneously at the single cell level (see FIG. 6 and detailed explanation of figure data in text below). It is possible to explore the heterogeneity of peripheral CD8 T_{SCM}, CD8 T_{CD57} and CD8 T_{CD25} cells specifically, and all CD8 T cells generally. The present invention can be used to identify the transcription factors that regulate CD8 T_{SCM}, CD8 T_{CD57}, and CD8 T_{CD25} cells identified. Using the present invention, it is functionally possible to interrogate CD8 T_{SCM}, CD8 T_{CD57}, and CD8 T_{CD25} from high- and low-CVD subjects for distinctions in proliferation, activation, differentiation, and cytokine production. Thus, using the present invention it is possible to distinguish CD8 T_{SCM}, and CD8 T_{CD57} cells as they differentiate into memory and effector subsets. Thus, it possible to phenotypically and functionally evaluate changes in human CD8 T cell populations during atheroprogression.

Study Design. Transcriptional and gene expression profiles of CD8 T_{SCM}, CD8 T_{CD57} and CD8 TCD25 cells and how do these gene expression profiles differ in low-versus high CVD individuals. It is also possible to identify other unique CD8 subsets in high-risk CVD subjects, includes whether there are sex or race/ethnic differences in CD8 T_{SCM}, CD8 T_{CD57}, and CD8 T_{CD25} cells.

CD8 T cell heterogeneity is not well understood in human CVD. Moreover, the transcriptional profiles of CD8 subsets in subjects with CVD are unknown. Unbiased, high-dimensional immunoprofiling approaches are used that feed directly into functional analyses of CD8 T cell subsets in humans with low- and high-risk CVD.

Study Design. Human subjects. Subjects in the MESA cohort with high versus low CVD-risk, as determined by CAC Agatson scoring is used. Using the present invention, it is also possible to examine sex and race/ethnicity differences in CD8 T cell populations and their links to atherosclerosis.

Single-cell transcriptional profiling (Abseq). A novel type of single-cell indexing of both transcriptomes and proteomes using the Rhapsody system (BD Biosciences) can be used to examine distinct profiles of the CD8 T cell compartment in human atherosclerosis. The Rhapsody platform measures protein and mRNA expression simultaneously in single cells using oligo-conjugated antibodies (Ab-Seq) in combination with massively parallel single-cell mRNA sequencing (scRNA-seq). This method circumvents common inconsistencies with analyzing both protein and mRNA expression of important surface markers at a single-cell resolution. Ab-Seq is also superior for detecting protein targets with relatively low mRNA levels, detecting cells with low transcriptional activity, and measuring transcription factor expression. Data illustrating the feasibility of performing Ab-Seq in human samples is shown in FIG. 14. In this experiment, ˜6600 PBMC from healthy subjects were analyzed with an antibody and gene panel that closely examined T cell subsets. In the left panel of FIG. 15, protein expression via heatmap is shown, and in the right panel, gene signatures of key genes in each CD8 subset identified in the left panel are shown. The combination of studying both proteins and genes simultaneously in single cells allows for more detailed identification of all CD8 subsets; hence the discovery of 14 CD8 subsets using BD Rhapsody. Based on the high dimensional data, the T_{CD25} subset is #6, the T_{N} is #2, the T_{SCM} is #9 and the T_{CD57} is #11 or #12; thus, illustrating feasibility for use of the present invention with MESA samples.

PBMCs from MESA subjects is thawed and enriched for CD8 T cells via magnetic separation. Sample tagging and pooled-antibody staining can be used to minimize batch effects, the BD Rhapsody system to generate single-cell mRNA libraries, and an in-house Nova Seq instrument (Illumina) for sequencing. Ab-Seq data is analyzed using integrated analyses that combine best practices for scRNA-seq and CyTOF, as well as the current bioinformatics pipeline from BD Biosciences. Specifically, the inventors can use the Seurat integrated framework₆₈, based on a pairwise canonical correlation analysis, to normalize batch effects and unwanted variables from single-cell data. Different clustering methods and statistical evaluations can also be used, which the inventors previously did for CyTOF₆₉, to identify the most stable CD8 T cell clustering, accounting for different protein and mRNA expression levels. These results identified important markers for each cellular subset that define the roles of these subsets in CVD progression through flow cytometry. Differentially expressed genes (DEGs) between high and low CVD-risk patients for CD8 T_{SCM}, CD8 T_{CD57}, and CD8 T_{CD25} cells and any other differentially abundant CD8 T cell subsets using best practices from a benchmark study of single-cell differential expression analysis can also be determined. Gene-set enrichment analysis can identify significantly enriched biological and cellular pathways within CD8 T cell subsets as well as those specific for high CVD-risk patients. Important transcriptional factors and their target genes using transcriptional regulation network analysis by the SCENIC method can also be determined. Identifying the transcription factors enriched in each new subset aids in identifying the biological output of these cells.

All DEGs identified can be validated by quantitative PCR. Populations called as T_{SCM}, CD8 T_{CD57}, and CD8 T_{CD25} cells and any other novel CD8 subsets identified by BD Rhapsody are validated in additional low- and high-risk CVD individuals from MESA using conventional flow cytometry (n=5 per group for validation). CD8 T_{SCM} cells are phenotyped according to the procedures detailed in Lugli et al., 2013⁷³. These findings are confirmed with additional CAVA cohort samples from UVA. Analyzing this replication cohort by conventional flow cytometry also provides numbers and frequencies of these CD8 T cell subsets to be linked with matched clinical data. It is possible to correlate data obtained from the Ab-Seq and flow cytometry-based validation experiments with clinical parameters for the MESA subjects, including plasma lipoprotein levels, oxLDL levels, and HbA1c levels (as a measure of diabetes). There are over 1000 clinical measurements for each MESA subject, allowing for extensive clinical correlations.

Using the present invention, it is possible to uncover novel surface markers and transcriptional signatures for the CD8 compartment. CD8 T_{SCM} cells are increased in MESA subjects with high CVD-risk (defined by high CAC Agatson scores). c-Myb, a transcription factor that regulates thymocyte development, T_{Reg} differentiation, and T cell stemness⁷⁴, is increased in T_{SCM} cells compared to other CD8 T cell populations. Transcriptomic analysis of whole and YF-specific T_{SCM} cells has revealed their close relationship to T_{CM} cells, and the inventors show transcription factors are shared between T_{SCM} and T_{CM} cells. The TCD25 subset expresses the transcription factor Foxp3, as this is the defining transcription factor for CD4 regulatory cells. The inventors also found DEG expression of exhaustion markers in the T_{CD57} subset. The CD57 subset may also possesses unique transcription factors. Ancestry-associated differences in CD8 populations were also found. This strategy for examining CD8₊ T cell heterogeneity within MESA individuals makes it possible to pinpoint the transcription factor network that governs CD8 T cell differentiation in atherosclerosis, which is currently undefined.

Alternative Strategies. As a follow-up or alternative strategy to scRNA-seq, it is possible to analyze CD8 T cell heterogeneity utilizing high-dimensional CyTOF. Briefly, one million PBMCs are stained with rare earth metal-conjugated antibodies using a validated panel for examining CD8₊ T cell heterogeneity. For example, the inventors can use the T cell panel employed in FIG. 11 and include known, suspected, or newly identified markers for identified populations and their possible activation or exhaustion, such as CD127, CD58, CXCR3, IL-2Rβ, CD11a, CD25, FoxP3, CD57, CXCR4, Lag-3, PD-1, CTLA-4, TIGIT, CD69, CD44, and CD28_{73, 77, 78}. Generated mass cytometry data are analyzed using an integrated computational pipeline.

MESA subjects were not on cholesterol-lowering or other heart medications prior to their enrollment in the cohort, thus statins or other medications are not a factor in this study. As such, it is possible to observe ancestry-associated differences in either CD8 T cells or CD8 subsets between Caucasians and African Americans, using PBMCs from the Jackson Heart Study. Within the MESA cohort, approximately 16% of high-CVD severity participants have reported an MI (79 high-risk CVD individuals). CVD patients with reported MIs were selected for BD Rhapsody-based analysis as a separate cohort. As CVD is frequently concurrent with many conditions₇₉₋₈₂, individuals are stratified based on comorbidities.

Functional differences in CD8 T_{SCM}, CD8 T_{CD57} and CD8 T_{CD25} cells in low- and high-risk CVD individuals. To functionally interrogate the CD8 subsets and any additional CD8 subsets identified in the functional studies focus on proliferation, exhaustion, activation, and cytokine production.

Study Design. Sort identified CD8 T cell subsets from additional matched PBMC from MESA subjects (n=10-15 per group, Table 4) for functional studies.

Analysis of CD8 T cell proliferation, activation or exhaustion. CD8 T_{N}, (CD8⁺CD3⁺CD45RA⁺CCR7⁺CD45RO⁻CD95⁻), T_{SCM} (CD8⁺CD3⁺CD45RA⁺CCR7⁺CD45RO⁻CD95⁺), T_{CM} (CD8⁺CD3⁺CD45RO⁺CD45RA⁻CCR7⁺), T_{EM} (CD8⁺CD3⁺CD45RO⁺CD45RA⁻CCR7⁻). CD8 T_{CD57} (CD8⁺CD3⁺CD45RA⁺CD57⁺), and CD8 T_{CD25} (CD8⁺CD3⁺CD25⁺) cells are sorted from low and high CVD-risk patients described in Table 4 and stained with Cell Trace Violet (CTV) to assess proliferation, as described prior²¹. Sorted cells are stimulated with anti-CD3/CD28 Dynabeads (Gibco) for 48 hours. Dilution of CTV is standardly used to assess proliferation₂₇. As controls for assessing the proliferative capacity of new CD8 subsets, CD8 T_{N}, T_{CM}, and T_{EM} cells are also be quantified. For analysis of activation or exhaustion, sorted T cell subsets above from low and high-risk CVD individuals are stimulated with anti-CD3/28 Dynabeads for 24, 48, and 72h or used unstimulated. T cell activation markers are assessed by flow cytometry for the following markers: CD25 (antibody clone BC96), CD38 (HB-7), CD69 (FN50), HLA-DR (L243). T cell exhaustion markers include PD-1 (EH12.2H7), CTLA-4 (BN13), Tim-3 (F38-2E2), Lag-3 (7H2C65), CD160 (BY55), and TIGIT (A15153G). The expression of the transcription factors Foxp3 (PCH101), t-bet, (4B10) and Eomes (WD1928 or Dan11mag) by flow cytometry can be assessed. Foxp3 is the hallmark transcription factor for T_{Reg} cells⁸⁶⁻⁸⁸ and T-bet_{89,90} and Eomes₉₁ are important transcriptional regulators of CD8 activation and differentiation. Assessments of proliferation and activation are performed simultaneously.

Measurement of cytokines and cytotoxic protein production. IFN-γ (4S.B3), TNF-α (Mab1), IL-2 (MQ1-17H12), IL-6 (MQ2-13A5), Granzyme B (QA16A02), and Perforin (B-D48) production by CD8 T cell subsets are assessed by intracellular cytokine staining (BD Biosciences) following stimulation with anti-CD3/28 Dynabeads. Data are then be analyzed using FlowJo v10.7.

T_{SCM} cells from high CVD-risk individuals display increased proliferative capabilities and pro-inflammatory cytokine production compared to T_{SCM} cells from low CVD-risk individuals.

Alternative Approaches. Male and female patients with high and low CVD-risk are evaluated. These CVD patients are part of an ongoing CAVA cohort at UVA, which includes patients 30-80 years of age undergoing medically necessary cardiac catheterization. Most of the CAVA subjects are Caucasian or African American. CAC scores within the CAVA cohort are measured in the same way as in the MESA cohort, allowing the inventors to readily interchange data between the two groups. CAVA samples can be obtained fresh or larger volumes of frozen cells can be obtained if needed for functional studies. Suppression assays can also be performed using autologous CD8 T_{CD25} and autologous T effector cells sorted from healthy human donors obtained from the LJI Normal Blood Donor Program to assess whether the CD8 T_{CD25} functions as a regulatory T cell, similar to CD4₊CD25₊ Tregs.

Test how CD8 T_{SCM}, CD8 T_{CD57}, and CD8 T_{CD25} cells impact atherosclerosis, defining the biological events linking CD8 T cell subsets to atherosclerosis. Well-characterized, atherosclerosis-prone mouse strains are used. Using the present invention, it is possible to determine the atherogenicity of CD8 T_{CD57}, and CD8 T_{CD25} cells, CD8 T_{CD25} cells are atheroprotective.

T cell subsets that impact atherosclerosis. Using the present invention, it is possible to adoptively transfer sorted CD8 T_{N}, T_{SCM}, T_{CM}, TCD57, TCD25, and T_{EM} cells, into immune-deficient Rag.Ldlr_{-/-} recipients fed a high-cholesterol diet (HCD) and then monitor atherosclerosis.

Adoptive Transfer. T_{SCM} cells were originally Identified in mice and defined as CD44^{low}CD62L^{high} T_{N} cells expressing stem cell antigen (Sca-1). As there is no human ortholog for Sca-1, T_{SCM} cells are phenotyped slightly differently in mice than in humans; however, T_{N} cells from both species express B cell lymphoma-2 (Bcl-2)^{27, 93}. CD8 T_{N} (CD62L^{high}CD44^{low}Sca-1^{low}), T_{SCM} (CD62L^{high}CD44^{low}Sca-1^{high}), CD8 T_{CD57} (CD57⁺), and CD8 T_{CD25} (CD25⁺) cells, along with control T_{EM} (CD62L^{low}CD44^{high}) and control T_{Reg} (CD4⁺CD25⁺GITR⁺) cells, are sorted from 12-week-old B6 lymph nodes and spleens by staining with antibodies against the following: CD57 (NK-1), CD4 (RM4-5), CD8 (53-5.8), CD25 (3C7), CD44 (IM7), CD62L (MEL-14), GITR (DTA-1), Sca-1 (D7), and TCRβ (H57-597). Additional markers can be used to sort TCD25 and TCD57. To minimize time spent sorting, which can negatively influence cell viability, single-cell suspensions are pre-enriched for CD3 T cells. Female B6 mice are utilized in female recipients and male B6 mice used in male recipients for all adoptive transfer studies to prevent cell rejection. 2×10⁵ CD8 T_{N}, TCD25, T_{SCM}, T_{CD57}, T_{EM} cells and 7×10⁴ T_{Reg} cells are injected intravenously into 12-week-old, lymphocyte-deficient Rag.Ldlr-recipients (n=15 mice per transfer group per sex; see Rigor). The rationale for co-transferring with T_{Reg} cells is to slow cell division and prevent recipient mice from dying due to robust homeostatic proliferation⁹⁴. As controls, Rag.Ldlr^{-/-} mice will receive CD8 T_{EM}, known to elicit atherosclerotic lesions, in addition to T_{Reg}-injected controls. Rag.Ldlr^{-/-} mice are utilized to ensure that the transferred CD8 T cells are the only adaptive immune cell present within the recipient. Prior to transfer, all recipient mice are bled and phenotyped to ensure that adaptive immune cells are absent. Mice are placed on an HCD containing 1.25% cholesterol and no sodium cholate₉₅ immediately following adoptive transfer. Mice are closely monitored and weighed twice weekly. As Bcl-2 is an intracellular marker, cannot be used to sorting CD8 T_{SCM} cells; nevertheless, Sca-1high T_{N} cells are considered CD8 T_{SCM} cells. Donor cell viability (aiming for >95%) is checked by trypan blue staining prior to adoptive transfer. Peripheral blood is collected from recipient mice at 3, 6, and 9 weeks post-adoptive transfer to confirm the presence of transferred T cells in recipient mice compared to Rag.Ldlr_{-/-} control mice. The inventors have established that adoptively transferred mice can survive long-term for these studies (Rag.Ldlr-mice have been followed out to approximately 4 months)_{11, 59}.

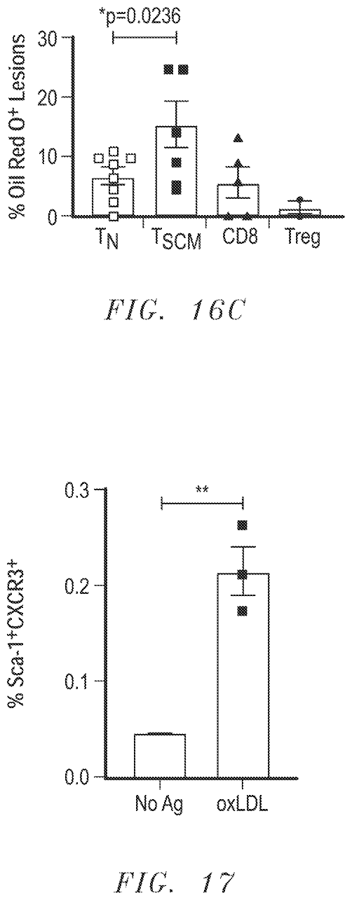

To support this hypothesis, and to demonstrate the feasibility of this work, CD8 T_{N}, T_{SCM}, and T_{EM} cells were transferred intravenously into 2 immune-deficient, Rag.Ldlr recipients (per group), which were fed an HCD for 12 weeks. In contrast to T_{N} and T_{SCM} cells, T_{EM} cells preferentially traffic to peripheral tissues, where they rapidly assume effector T cell functions. Interestingly, analysis of aortic roots at 12 weeks post HCD revealed a marked increase in Oil Red O₊ cells in T_{SCM} transfer recipients, indicative of enhanced atherosclerosis severity compared to CD8 T_{N} or T_{EM} recipients (FIG. 16). This increase in atherosclerotic plaque formation was accompanied by no significant changes in weight loss within T_{N}, T_{SCM}, or T_{EM} cell transfer recipients. CD4 Treg are co-transferred to prevent colitis⁹⁶. These data show that CD8 T_{SCM} cells are proatherogenic.

Atherosclerosis Quantification. Atherosclerosis severity can be assessed by enface and aortic root analysis, as these two methods do not always give identical results₆₂. These tissues can be studied histologically, including Movats and Picrosirius Red stains to examine lesion composition, in terms of extracellular matrix and collagen content, respectively. Lesion necrotic core content is assessed by TUNEL staining, as described prior_{97, 98}. Plasma lipoprotein measurement is performed using FPLC as described₅₉. Plasma is collected from all mice for quantification of cytokines, including TNFα, IL-1β, and IL-18 by multiplexed ELISA.

Measurement of CD8 T cell subset proliferation in mouse aortas. Aortas from a second group of mice are harvested for flow cytometry-derived detection of CD8 T_{SCM} proliferation in the aorta. As atherosclerosis measurements and flow cytometry measurements cannot be perform in the same tissue simultaneously. Aortas are processed quickly to maintain T cell viability. The flow cytometry panel will consist of live/dead dye and antibodies against Bcl-2, CD8, CD44, CD45, CD62L, Ki67 (11F6), CD57, CD25, CD3, Sca-1, and TCRβ. The amount of Ki-67 positivity of T cells in flow cytometry is a measurement of proliferation.

T cell subset Immunophenotyping. In adoptively-transferred Rag.Ldlr recipients, the inventors analyzed the CD8 T cell compartment within blood, inguinal lymph nodes, and paLNs, via conventional 18-color flow cytometry, utilizing 15 mice per group. This analysis is performed separately from the atherosclerosis above because both cytometry and atherosclerosis cannot be measured in the same mouse, as the aorta must be digested for flow cytometry. Aorta, paLNs, and inguinal lymph nodes are stained with antibodies to detect CD8 T_{N}, T_{SCM}, T_{EM}, T_{CD25}, and T_{CD57} cells, in addition to macrophages, monocytes, and B cells. The flow panel for para-aortic and inguinal lymph node staining includes: Bcl-2 (BCL/10C4), CD8, CD11b (M1/70), CD19 (4G7), CD44 (IM7), CD62L, CD86 (GL-1), CD115 (AFS98), F4/80 (BM8), Ly6C (HK 1.4), Ly6G (1A8), Sca-1, TCRβ, CD57 (NK-1), CD25, (3C7) and live/dead dye, while the blood is stained with the following antibody cocktail: CD3, CD4, CD8a, CD11b, CD19, CD62L, CD44, CD45, CD115, Ly6C, Ly6G, NK1.1, TCRβ, and live/dead dye. For power calculations, utilizing nQuery advisor 6.0, 90% power with type 1 error can be achieved with 15 mice per group for flow cytometry.

As shown in Example 1, adoptive transfer of T_{SCM} cells increases atherosclerosis. Adoptive transfer of CD8 T_{SCM} cells elicit increased pro-inflammatory monocytes within the blood and paLNs, concomitant with increased atherosclerotic plaque formation, as previously demonstrated by Kyaw et. al₅. The proliferative marker Ki67 is enhanced within the T_{SCM} adoptively-transferred recipients compared to T_{N}, T_{EM}, or bulk CD8 T cell recipients. CD8 T_{SCM} cells are highly proliferative and thus skewed T_{CM} and T_{EM} frequencies arise within transferred recipients. Based on the results herein, the CD8 T_{CD57} cells are proatherogenic. CD8 T_{CD25} cells are atheroprotective, like their CD4 T_{Reg} counterparts.

Alternative Approaches. Adoptive transfers into Rag.Ldlr_{-/-} recipients are technically challenging, as the transferred cells undergo homeostatic proliferation and animals must be kept on an HCD for at least 12 weeks for sufficient atherosclerotic plaque formation. It is possible to sort cells from multiple B6 donor mice in order to obtain enough CD8 T_{CD57} and CD8 T_{CD25} cells to inject. As an alternative approach for the CD8 T_{SCM} adoptive transfer studies, the inventors can use sub-lethally irradiated Ldlr_{-/-} mice and deplete CD8 T cells via CD8 T cell depletion antibodies (Bioxcell). 7-week-old Ldlr_{-/-} mice (B6.129S7-Ldlr_{tm1Her}, Stock: 002207) are sub-lethally irradiated with 550 rad 2-4 hours apart. On the day of irradiation, mice are injected i.p. with 200 μg anti-mouse CD8alpha (clone 53-6.7) and anti-CD8beta (clone 53-5.8) depleting antibodies and transferred i.v. with 5 million CD8_{-/-} bone marrow cells from CD8_{-/-} mice (CD8a_{tm1Mak}) retro-orbitally₁₀₀. Three days later CD8_{-/-} chimeric mice receive another 200 μg i.p. dose of the anti-CD8 depleting antibodies. At week 10, chimeric mice receive CD8 T_{N}, T_{SCM}, TCD57, TCD25, or T_{EM} (2×10⁵) cells i.v. and fed a high cholesterol diet for approximately 15 weeks.

As an additional alternative approach for CD8 T cell transfer, to utilize human CD8 T cells, PBMC-sorted CD8 T_{N} (CD8⁺CD45RA⁺CCR7CD95^{low}), T_{SCM}(CD8⁺CD45RA⁺CCR7CD95^{high}), T_{EM} (CD8⁺CD45RA⁻CCR7⁺), CD8 T_{CD57} (CD8⁺CD3⁺CD45RA⁺CD57⁺), and CD8 T_{CD25} (CD8⁺CD3⁺CD25⁺) cells from individuals participating in the Healthy Blood Donor Program at LJI are transferred into humanized NOD.scid gamma (NOD.Cg-Prkdc_{scid}Il2rg_{tm1Wj1}/SzJ mice) (NSG) recipients (Stock: 005557, JAX) via intravenous injection. Mice in this case would be subsequently injected with an adeno-associated virus (AAV) vector that supports targeted transfer of human proprotein convertase subtilisin/kexin type 9 (hPCSK9), which elicits robust atherosclerotic plaque formation¹⁰¹. The adoptive transfer of 3×10⁶ CD8 T_{SCM} cells into humanized NSG mice has been previously performed, and mice were aged for approximately 40 weeks post-transfer. Recipient, humanized NSG mice are closely monitored for weight loss following adoptive transfer. As an additional alternative approach, BrdU can be injected to track the proliferating cells as described₆₉.

Using the present invention its possible to determine CD8 T_{SCM} cells from atherosclerotic mice display increased differentiation into central and effector memory subsets, if changes in CD8 T_{CD25}, or TCD57 cell subsets in atherosclerotic mice, and does oxidized LDL promote increased differentiation of CD8 T_{N} cells into these novel T cell subsets.

CD8 T_{SCM} cells are highly proliferative and self-renewing, able to rapidly differentiate into central and effector memory subsets²⁷. Using the teachings herein the inventors can study the ability of T_{N} cells to differentiate into T_{SCM} cells during atherosclerosis progression, and even examine the fate of T_{SCM} in atherosclerosis. T_{N} cells more rapidly differentiate into T_{SCM} cells and that T_{SCM} cells more quickly differentiate into T_{CM} and T_{EM} CD8 T cell subsets with HCD-feeding. The inventors observed increased T_{CD57} cells in subjects with high CVD, thus, WD or oxidized LDLs increase their differentiation. CD4 T_{Reg} cells (CD25⁺Foxp3⁺) lose expression of Foxp3 and obtain effector functions in atherosclerosis. This may account for some of the loss of the T_{CD25} subset observed in high CVD subjects in FIG. 14. It is possible to discern how oxLDL impacts T_{CD25} differentiation.

Study Design. Adoptive transfers to address a) whether T_{SCM} become T_{CM} or T_{EM} in atherosclerosis and b) whether T_{N} become T_{CD57} or T_{CD25} in atherosclerosis. T_{N} and T_{SCM} cells are sorted from chow-fed CD45.1-Apoe^{-/-} mice and 2×10₅ cells are transferred intravenously into chow and WD-fed Apoe_{-/-} (B6.129P2-Apoe_{tm1Un}) recipients. At 7- and 21-days post-injection, aortas, blood, LNs (axillary, brachial, cervical, inguinal, and para-aortic), and spleens are collected, and flow cytometry is performed on these samples to detect T_{N} (CD44^{low}CD62L^{high}Sca-1^{low}), T_{SCM} (CD44^{low}CD62L^{high}Sca-1^{high}), T_{CM} (CD44^{high}CD62L^{high}), T_{EM} (CD44^{high}CD62L^{low}), T_{CD57} (CD8⁺CD57⁺), and T_{CD25} (CD8⁺CD25⁺) cells. If expression of Foxp3 or Eomes in the T_{CD57} and T_{CD25} subsets, these markers can be included in subsequent flow panels. The rationale for selecting these time points is that there is an early expansion phase at 7 days, but also that by 21 days effector memory differentiation is increased within adoptively-transferred recipients. CD8 T_{N}, T_{SCM}, T_{CM}, T_{EM}, cells are detected in aorta, blood, LNs and spleen by staining with Bcl-2 (BCL/10C4), CD8 (53-5.7), CD44 (IM7), CD45 (30-F11), CD45.1 (A20), CD45.2 (104),

CD62L (MEL-14), CXCR3 (CXCR3-173), Sca-1 (D7), Foxp3 (FJK-16S), Eomes (Dan11 mag), and TCRβ (H3-597).

Suppressive T_{CD25} cells. Conventional T_{Reg} (CD4⁺CD25⁺) and CD8 T_{CD25} cells are sorted from spleens and peripheral LNs of B6 mice (these mice are CD45.2₊, allowing the inventors to use both CD45.1 and CD45.2 alleles in this experiment to distinguish CD45.2⁺ T_{Reg} and T_{CD25} from CD45.1⁺ effector cells). Naïve effector (CD4⁺CD25⁻ CD44ₗₒCD62Lₕᵢ) cells are sorted from spleens and peripheral LNs of CD45.1⁺ mice and labeled with CTV according to manufacturer's instruction. Feeder cells are prepared from spleens of B6 mice and CD4 and CD8 T cells depleted using CD3₊ magnetic beads (Miltenyi Biotec, Auburn, Calif.) and then are irradiated with 3000 rads. Feeders are cultured with naïve CD45.1₊ effector T cells at a ratio of 3:1 in U-shaped 96 well plates. Sorted T_{Reg} or CD8 T_{CD25} from B6 mice (CD45.2₊) are added at a ratio of 1:2, 1:4, or 1:8. Cells are stimulated with soluble αCD3 antibody at 2.5 μg/ml, and cultured in RPMI media supplemented with 10% FCS, L-glutamine, penicillin and streptomycin, P-mercaptoethanol. Cells are harvested 4 days following stimulation and the percentage of effector CD45.1⁺ cells that show diluted CTV is determined by flow cytometry.

Role of OxLDL in the differentiation of CD8 T_{N} into effector subsets. As the least differentiated memory subset, a hallmark of T_{SCM} cells is their ability to rapidly differentiate into more advanced memory cells, such as T_{CM} or T_{EM} cells. Whether oxLDL, a major immunomodulatory component of the atherosclerotic plaque, impacts CD8 T_{N} differentiation into these other effector subsets as shown in FIG. 17 and in Example 1 can be determined. FIG. 17 shows that T_{N} cells from chow-fed Apoe−/− mice cultured with 10 μg/mL oxLDL elicits a skewing of CD8 T_{N} toward T_{SCM} cells. Negatively-selected, splenic naive CD8 T cells from ApoE_{-/-} mice are added to a 24-well plate pre-coated overnight with 0.1 μg/mL anti-CD3, 1 μg/mL anti-CD28 (BD Biosciences) and blocked with media for at least 3 hours at room temperature. CD8 T cells are stimulated in serum-free CST OpTimizer T cell expansion media (Thermo Fisher Scientific), supplemented with L-glutamine, penicillin and streptomycin, and b-mercaptoethanol with anti-CD3, CD28 under T helper 0 (Th0), T helper 1 (Th1: 10 μg/mL anti-IL-4 (Biolegend), 10 ng/mL rmIL-12p70 (Peprotech)), T helper 2 (Th2:), T helper 17 (Th17: 10 μg/mL anti-IL-4 (11B11), 10 μg/mL anti-IFN-g (XMG1.2; Biolegend), 25 ng/mL rmTL-6 (Peprotech), rmIL-1b, rhTGF-b (R&D)), T_{CD25} (T_{Reg}: 5 ng/mL rhTGF-b 50U/mL IL-2 (Peprotech)) and T_{SCM} (IL-7 and IL-15 (R&D); 25 ng/mL)²′ conditions for 72 hr at 37° C. with 5% CO₂, in the presence or absence of 10 μg/mL oxLDL (Kalen Biomedical)_{58,59,102,103}.

With WD-feeding, T_{SCM} differentiation into T_{CM} (CD8⁺CD44^{high}CD62L^{high}) and T_{EM} (CD8⁺CD44^{high}CD62L^{low}) subsets is increased. Adoptive transfer of CD45.1-labelled T_{SCM} cells into Apoe^{-/-} mice will elicit increased formation of T_{CM} and T_{EM} subsets with WD-feeding compared to chow-feeding. At 21 days post-transfer the majority of CD45.1⁺ cells will possess a T_{EM}-like phenotype. Importantly, adoptive transfer of T_{N} cells will elicit increase T_{SCM} formation under WD-feeding. Lowered suppressive activity of T_{CD25} in atherosclerotic conditions is observed, and the development of higher frequencies of T_{CD57} cells in the presence of oxLDL.

Using the present invention, it is also possible to detect adoptively transferred subsets based on their CD45.1 expression. T_{N} cells can be cultured with dil(1,1′-dioctadecyl-3,3,3′,3′-tetramethylindocarbocyanine perchlorate)-oxLDL and determine if the cells that have taken up oxLDL (PE₊ cells) are biased towards a certain T cell phenotype. Additionally, human T_{N} CD8 T cells from healthy donors can be sorted and incubated with oxLDL or dil-oxLDL for 72 hours at 37° C. and determine if culture induces these T cell phenotypes. A bias towards T_{CM} and T_{EM} phenotypes during oxLDL incubation suggests that CD8 T_{SCM} cells recognize processed antigens within the atherosclerotic plaque, thus making it possible to identify the antigenic peptide that drives this process.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ¬±1, 2, 3, 4, 5, 6, 7, 10, 12 or 15%.

Additionally, the section headings herein are provided for consistency with the suggestions under 37 CFR 1.77 or otherwise to provide organizational cues. These headings shall not limit or characterize the invention(s) set out in any claims that may issue from this disclosure. Specifically and by way of example, although the headings refer to a “Field of Invention,” such claims should not be limited by the language under this heading to describe the so-called technical field. Further, a description of technology in the “Background of the Invention” section is not to be construed as an admission that technology is prior art to any invention(s) in this disclosure. Neither is the “Summary” to be considered a characterization of the invention(s) set forth in issued claims. Furthermore, any reference in this disclosure to “invention” in the singular should not be used to argue that there is only a single point of novelty in this disclosure. Multiple inventions may be set forth according to the limitations of the multiple claims issuing from this disclosure, and such claims accordingly define the invention(s), and their equivalents, that are protected thereby. In all instances, the scope of such claims shall be considered on their own merits in light of this disclosure, but should not be constrained by the headings set forth herein.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

To aid the Patent Office, and any readers of any patent issued on this application in interpreting the claims appended hereto, applicants wish to note that they do not intend any of the appended claims to invoke paragraph 6 of 35 U.S.C. ¬ß 112, U.S.C. ¬ß 112 paragraph (f), or equivalent, as it exists on the date of filing hereof unless the words “means for” or “step for” are explicitly used in the particular claim.

For each of the claims, each dependent claim can depend both from the independent claim and from each of the prior dependent claims for each and every claim so long as the prior claim provides a proper antecedent basis for a claim term or element.

### REFERENCES—EXAMPLE 1

Benjamin E J, et al., American Heart Association Council on Epidemiology and Prevention Statistics Committee and Stroke Statistics Subcommittee. Heart Disease and Stroke Statistics-2019 Update: A Report From the American Heart Association. Circulation. 2019; 139:e56-e528.
Mallat Z, Taleb S, Ait-Oufella H, Tedgui A. The role of adaptive T cell immunity in atherosclerosis. J Lipid Res. 2009; 50 Suppl:S364-9.
Tedgui A, Mallat Z. Cytokines in atherosclerosis: pathogenic and regulatory pathways. Physiol Rev. 2006; 86:515-581.
Whitman S C, Ravisankar P, Elam H, Daugherty A. Exogenous Interferon-7 Enhances Atherosclerosis in Apolipoprotein E−/− Mice [Internet]. The American Journal of Pathology. 2000; 157:1819-1824. Available from: http://dx.doi.org/l0.1016/s0002-9440(10)64820-1
Ait-Oufella H, et al., Natural regulatory T cells control the development of atherosclerosis in mice. Nat Med. 2006; 12:178-180.
Kyaw T, et al., Cytotoxic and Proinflammatory CD8 T Lymphocytes Promote Development of Vulnerable Atherosclerotic Plaques in ApoE-Deficient Mice [Internet]. Circulation. 2013; 127:1028-1039. Available from: http://dx.doi.org/10.1161/circulationaha.112.001347
Olofsson et al., CD137 is expressed in human atherosclerosis and promotes development of plaque inflammation in hypercholesterolemic mice. Circulation. 2008; 117:1292-1301.
Kolbus D, et al., CD8+ T cell activation predominate early immune responses to hypercholesterolemia in Apoe⁻(/)⁻ mice. BMC Immunol. 2010; 11:58.
Elhage R, et al., Deleting TCRαβ or CD4 T Lymphocytes Leads to Opposite Effects on Site-Specific Atherosclerosis in Female Apolipoprotein E-Deficient Mice [Internet]. The American Journal of Pathology. 2004; 165:2013-2018. Available from: http://dx.doi.org/10.1016/s0002-9440(10)63252-x
Kolbus D, et al., TAP1-Deficiency Does Not Alter Atherosclerosis Development in Apoe−/− Mice [Internet]. PLoS ONE. 2012; 7:e33932. Available from: http://dx.doi.org/10.1371/journal.pone.0033932
Gewaltig J, et al., Requirements for CD8 T-cell migration into the human arterial wall. Hum Pathol. 2008; 39:1756-1762.
van Dijk R A, et al., A Change in Inflammatory Footprint Precedes Plaque Instability: A Systematic Evaluation of Cellular Aspects of the Adaptive Immune Response in Human Atherosclerosis [Internet]. Journal of the American Heart Association. 2015; 4. Available from: http://dx.doi.org/10.1161/jaha.114.001403
Paul V S V. Quantification of Various Inflammatory Cells in Advanced Atherosclerotic Plaques [Internet]. JOURNAL OF CLINICAL AND DIAGNOSTIC RESEARCH. 2016; Available from: http://dx.doi.org/10.7860/jcdr/2016/19354.7879
Paul V S V, Paul C M P, Kuruvilla S. Quantification of Various Inflammatory Cells in Advanced Atherosclerotic Plaques. J Clin Diagn Res. 2016; 10:EC35-8.
Grivel J-C, et al., Activation of T lymphocytes in atherosclerotic plaques. Arterioscler Thromb Vasc Biol. 2011; 31:2929-2937.
Tae Yu H, et al., Characterization of CD8(+)CD57(+) T cells in patients with acute myocardial infarction. Cell Mol Immunol. 2015; 12:466-473.
Bergström I, et al., CD8 CD56 T cells that accumulate in patients with coronary artery disease are characterized by a pro-inflammatory cytokine profile [Internet]. Atherosclerosis. 2014; 235:e86. Available from: http://dx.doi.org/10.1016/j.atherosclerosis.2014.05.225
Hwang Y, et al., Expansion of CD8 T cells lacking the IL-6 receptor a chain in patients with coronary artery diseases (CAD) [Internet]. Atherosclerosis. 2016; 249:44-51. Available from: http://dx.doi.org/10.1016/j.atherosclerosis.2016.03.038
Gattinoni L, et al., T memory stem cells in health and disease. Nat Med. 2017; 23:18-27.
Becht E, et al., Dimensionality reduction for visualizing single-cell data using UMAP. Nat Biotechnol [Internet]. 2018; Available from: http://dx.doi.org/10.1038/nbt.4314
Olson N C, et al., Decreased naive and increased memory CD4(+) T cells are associated with subclinical atherosclerosis: the multi-ethnic study of atherosclerosis. PLoS One. 2013; 8:e71498.
Mor A, et al., Altered status of CD4(+)CD25(+) regulatory T cells in patients with acute coronary syndromes. Eur Heart J 2006; 27:2530-2537.
Cheng X, Y et al., The Th17/Treg imbalance in patients with acute coronary syndrome. Clin Immunol. 2008; 127:89-97.
Emoto T, et al., Regulatory/effector T-cell ratio is reduced in coronary artery disease. Circ J. 2014; 78:2935-2941.
Farber D L, Yudanin N A, Restifo N P. Human memory T cells: generation, compartmentalization and homeostasis. Nat Rev Immunol. 2014; 14:24-35.
Gattinoni L, et al., Wnt signaling arrests effector T cell differentiation and generates CD8+ memory stem cells. Nat Med. 2009; 15:808-813.
Vignali D, et al., Detection and Characterization of CD8 Autoreactive Memory Stem T Cells in Patients With Type 1 Diabetes. Diabetes. 2018; 67:936-945.
Hosokawa K, et al., J G 6th, Kaplan M J, Nussenblatt R B, Barrett A J, O'Shea J, Young N S. Memory Stem T Cells in Autoimmune Disease: High Frequency of Circulating CD8⁺ Memory Stem Cells in Acquired Aplastic Anemia. J Immunol. 2016; 196:1568-1578.
Lee Y J, et al., Role of Stem Cell-Like Memory T Cells in Systemic Lupus Erythematosus. Arthritis Rheumatol. 2018; 70:1459-1469.
Lugli E, et al., Identification, isolation and in vitro expansion of human and nonhuman primate T stem cell memory cells. Nat Protoc. 2013; 8:33-42.
Lugli E, et al., Superior T memory stem cell persistence supports long-lived T cell memory. J Clin Invest. 2013; 123:594-599.
Zhang Y, et al., Host-reactive CD8 memory stem cells in graft-versus-host disease [Internet]. Nature Medicine. 2005; 11:1299-1305. Available from: http://dx.doi.org/l0.1038/nm1326
Meir K S, Leitersdorf E. Atherosclerosis in the Apolipoprotein E-Deficient Mouse [Internet]. Arteriosclerosis, Thrombosis, and Vascular Biology. 2004; 24:1006-1014. Available from: http://dx.doi.org/10.1161/01.atv.0000128849.12617.f4
Wilhelm A J, Rhoads J P, Wade N S, Major A S. Dysregulated CD4+ T cells from SLE-susceptible mice are sufficient to accelerate atherosclerosis in LDLr−/− mice. Ann Rheum Dis. 2015; 74:778-785.
Zidar D A, et al., Altered Maturation Status and Possible Immune Exhaustion of CD8 T Lymphocytes in the Peripheral Blood of Patients Presenting With Acute Coronary Syndromes. Arterioscler Thromb Vasc Biol. 2016; 36:389-397.
Podolec J, et al., Higher levels of circulating naïve CD8CD45RA cells are associated with lower extent of coronary atherosclerosis and vascular dysfunction. Int J Cardiol. 2018; 259:26-30.
Beier C P, Schulz J B. CD95/Fas in the brain—not just a killer. Cell Stem Cell. 2009; 5:128-130.
Alizadeh D, et al., IL15 Enhances CAR-T Cell Antitumor Activity by Reducing mTORC1 Activity and Preserving Their Stem Cell Memory Phenotype. Cancer Immunol Res. 2019; 7:759-772.
Cochain C, et al., CD8+ T Cells Regulate Monopoiesis and Circulating Ly6C-high Monocyte Levels in Atherosclerosis in Mice. Circ Res. 2015; 117:244-253.
Writing Group Members, Mozaffarian D, et al., American Heart Association Statistics Committee, Stroke Statistics Subcommittee. Heart Disease and Stroke Statistics-2016 Update: A Report From the American Heart Association. Circulation. 2016; 133:e38-360.
Kern M J. The Cardiac Catheterization Handbook. Elsevier Health Sciences; 2003.
Manichaikul A, et al., A functionally significant polymorphism in ID3 is associated with human coronary pathology. PLoS One. 2014; 9:e90222.
Thomas G D, et al., Human Blood Monocyte Subsets: A New Gating Strategy Defined Using Cell Surface Markers Identified by Mass Cytometry. Arterioscler Thromb Vasc Biol. 2017; 37:1548-1558.
Hamers A A J, et al., Human Monocyte Heterogeneity as Revealed by High-Dimensional Mass Cytometry. Arterioscler Thromb Vasc Biol. 2019; 39:25-36.
Nowicka M, et al., CyTOF workflow: differential discovery in high-throughput high-dimensional cytometry datasets [Internet]. F1000Research. 2017; 6:748. Available from: http://dx.doi.org/10.12688/f1000research.11622.2
Van Gassen S, et al., FlowSOM: Using self-organizing maps for visualization and interpretation of cytometry data [Internet]. Cytometry Part A. 2015; 87:636-645. Available from: http://dx.doi.org/10.1002/cyto.a.22625.
Wilkerson M D, Neil Hayes D. ConsensusClusterPlus: a class discovery tool with confidence assessments and item tracking [Internet]. Bioinformatics. 2010; 26:1572-1573. Available from: http://dx.doi.org/10.1093/bioinformatics/btq170
Weber L M, et al., diffcyt: Differential discovery in high-dimensional cytometry via high-resolution clustering. Commun Biol. 2019; 2:183.
Gaddis D E, et al., Apolipoprotein AI prevents regulatory to follicular helper T cell switching during atherosclerosis. Nat Commun. 2018; 9:1095.
Galkina E, et al., Lymphocyte recruitment into the aortic wall before and during development of atherosclerosis is partially L-selectin dependent. J Exp Med. 2006; 203:1273-1282.

### REFERENCES—EXAMPLE 2

1. Tedgui A and Mallat Z. Cytokines in atherosclerosis: pathogenic and regulatory pathways. Physiol Rev. 2006; 86:515-81.
2. Ait-Oufella H, et al., Natural regulatory T cells control the development of atherosclerosis in mice. Nat Med. 2006; 12:178-80.
3. van Duijn J, Kuiper J and Slutter B. The many faces of CD8+ T cells in atherosclerosis. Curr Opin Lipidol. 2018; 29:411-416.
4. Elhage R, et al., Deleting TCR alpha beta+ or CD4+ T lymphocytes leads to opposite effects on site-specific atherosclerosis in female apolipoprotein E-deficient mice. Am J Pathol. 2004; 165:2013-8.
5. Kyaw T, et al., and proinflammatory CD8+ T lymphocytes promote development of vulnerable atherosclerotic plaques in apoE-deficient mice. Circulation. 2013; 127:1028-39.
6. Weber C, Zernecke A and Libby P. The multifaceted contributions of leukocyte subsets to atherosclerosis: lessons from mouse models. Nat Rev Immunol. 2008; 8:802-15.
7. Gewaltig J, et al., Requirements for CD8 T-cell migration into the human arterial wall.

Hum Pathol. 2008; 39:1756-62.
8. Grivel J C, et al., Activation of T lymphocytes in atherosclerotic plaques. Arterioscler Thromb Vasc Biol. 2011; 31:2929-37.
9. Manichaikul A, et al., A functionally significant polymorphism in ID3 is associated with human coronary pathology. PLoS One. 2014; 9:e90222.
10. Sullivan D R, Marwick T H and Freedman S B. A new method of scoring coronary angiograms to reflect extent of coronary atherosclerosis and improve correlation with major risk factors. Am Heart J. 1990; 119:1262-7.
11. Padgett L E, et al., Naive CD8(+) T Cells Expressing CD95 Increase Human Cardiovascular Disease Severity. Arterioscler Thromb Vasc Biol. 2020:ATVBAHA120315106.
12. Lopez-Verges S, et al., CD57 defines a functionally distinct population of mature NK cells in the human CD56dimCD16+NK-cell subset. Blood. 2010; 116:3865-74.
13. Osawa K, Nakanishi R and Budoff M. Coronary Artery Calcification. Glob Heart. 2016; 11:287-293.
14. Criqui M H, et al., Calcium density of coronary artery plaque and risk of incident cardiovascular events. JAMA. 2014; 311:271-8.
15. Chistiakov D A, Orekhov A N and Bobryshev Y V. Immune-inflammatory responses in atherosclerosis: Role of an adaptive immunity mainly driven by T and B cells. Immunobiology. 2016; 221:1014-33.
16. Spitz C, Winkels H, et al., Regulatory T cells in atherosclerosis: critical immune regulatory function and therapeutic potential. Cell Mol Life Sci. 2016; 73:901-22.
17. van Dijk R A, et al., A change in inflammatory footprint precedes plaque instability: a systematic evaluation of cellular aspects of the adaptive immune response in human atherosclerosis. J Am Heart Assoc. 2015; 4.
18. Chyu K Y, et al., CD8+ T cells mediate the athero-protective effect of immunization with an ApoB-100 peptide. PLoS One. 2012; 7:e30780.
19. Fyfe A I, Qiao J H and Lusis A J. Immune-deficient mice develop typical atherosclerotic fatty streaks when fed an atherogenic diet. J Clin Invest. 1994; 94:2516-20.
20. Zhou J, et al., CD8(+)CD25(+) T cells reduce atherosclerosis in apoE(−/−) mice. Biochemical and biophysical research communications. 2014; 443:864-70.
21. Bergstrom I, et al., Persistent accumulation of interferon-gamma-producing CD8+CD56+ T cells in blood from patients with coronary artery disease. Atherosclerosis. 2012; 224:515-20.
22. Tae Yu H, et al., Characterization of CD8(+)CD57(+) T cells in patients with acute myocardial infarction. Cell Mol Immunol. 2015; 12:466-73.
23. Kolbus D, et al., Association between CD8+ T-cell subsets and cardiovascular disease. J Intern Med. 2013; 274:41-51.
24. Farber D L, Yudanin N A and Restifo N P. Human memory T cells: generation, compartmentalization and homeostasis. Nat Rev Immunol. 2014; 14:24-35.
25. Gattinoni L, et al., Wnt signaling arrests effector T cell differentiation and generates CD8⁺ memory stem cells. Nat Med. 2009; 15:808-13.
26. Lugli E, et al., Superior T memory stem cell persistence supports long-lived T cell memory. J Clin Invest. 2013; 123:594-9.
27. Gattinoni L, et al., A human memory T cell subset with stem cell-like properties. Nat Med. 2011; 17:1290-7.
28. Gattinoni L, et al., T memory stem cells in health and disease. Nat Med. 2017; 23:18-27.
29. Cieri N, et al., IL-7 and IL-15 instruct the generation of human memory stem T cells from naive precursors. Blood. 2013; 121:573-84.
30. Sabatino M, et al., Generation of clinical-grade CD19-specific CAR-modified CD8+ memory stem cells for the treatment of human B-cell malignancies. Blood. 2016; 128:519-28.
31. Vignali D, et al., Detection and Characterization of CD8(+) Autoreactive Memory Stem T Cells in Patients With Type 1 Diabetes. Diabetes. 2018; 67:936-945.
32. Hosokawa K, et al., Memory Stem T Cells in Autoimmune Disease: High Frequency of Circulating CD8+ Memory Stem Cells in Acquired Aplastic Anemia. J Immunol. 2016; 196:1568-78.
33. Lee Y J, et al., Role of Stem Cell-Like Memory T Cells in Systemic Lupus Erythematosus. Arthritis Rheumatol. 2018; 70:1459-1469.
34. Bostrom K. Insights into the mechanism of vascular calcification. Am J Cardiol. 2001; 88:20E-22E.
35. Ruiz J L, et al., Zooming in on the genesis of atherosclerotic plaque microcalcifications. J Physiol. 2016; 594:2915-27.
36. Cardoso L and Weinbaum S. Changing views of the biomechanics of vulnerable plaque rupture: a review. Ann Biomed Eng. 2014; 42:415-31.
37. Kelly-Arnold A, et al., Revised microcalcification hypothesis for fibrous cap rupture in human coronary arteries. Proc Natl Acad Sci USA. 2013; 110:10741-6.
38. Maldonado N, et al., A mechanistic analysis of the role of microcalcifications in atherosclerotic plaque stability: potential implications for plaque rupture. Am J Physiol Heart Circ Physiol. 2012; 303:H619-28.
39. Rambhia S H, et al., Microcalcifications increase coronary vulnerable plaque rupture potential: a patient-based micro-C T fluid-structure interaction study. Ann Biomed Eng. 2012; 40:1443-54.
40. Budoff M J, et al., Progression of coronary calcium and incident coronary heart disease events: MESA (Multi-Ethnic Study of Atherosclerosis). J Am Coll Cardiol. 2013; 61:1231-9.
41. Detrano R, et al., Coronary calcium as a predictor of coronary events in four racial or ethnic groups. N Engl J Med. 2008; 358:1336-45.
42. Budoff M J, et al., Progression of coronary artery calcium predicts all-cause mortality. JACC Cardiovasc Imaging. 2010; 3:1229-36.
43. Arad Y, et al., Coronary calcification, coronary disease risk factors, C-reactive protein, and atherosclerotic cardiovascular disease events: the St. Francis Heart Study. J Am Coll Cardiol. 2005; 46:158-65.
44. Bild D E, et al., Multi-Ethnic Study of Atherosclerosis: objectives and design. Am J Epidemiol. 2002; 156:871-81.
45. Burke G, Lima J, Wong N D and Narula J. The Multiethnic Study of Atherosclerosis.

Glob Heart. 2016; 11:267-268.
46. Kondo T, et al., In Vitro Conversion of Activated T Cells into Stem Cell Memory-Like T Cells. Methods Mol Biol. 2019; 2048:41-51.
47. Yan C, et al., Memory stem T cells generated by Wnt signaling from blood of human renal clear cell carcinoma patients. Cancer Biol Med. 2019; 16:109-124.
48. Alizadeh D, et al., IL15 Enhances CAR-T Cell Antitumor Activity by Reducing mTORC1 Activity and Preserving Their Stem Cell Memory Phenotype. Cancer Immunol Res. 2019; 7:759-772.
49. Guan L, et al., Antigen-specific CD8+ memory stem T cells generated from human peripheral blood effectively eradicate allogeneic targets in mice. Stem Cell Res Ther. 2018; 9:337.
50. Pilipow K, et al., Antioxidant metabolism regulates CD8+ T memory stem cell formation and antitumor immunity. JCI Insight. 2018; 3.
51. Chen Y, et al., Adoptive Transfer of Interleukin-21-stimulated Human CD8+ T Memory Stem Cells Efficiently Inhibits Tumor Growth. J Immunother. 2018; 41:274-283.
52. Zhang J Y, et al., Modulation of CD8(+) memory stem T cell activity and glycogen synthase kinase 3beta inhibition enhances anti-tumoral immunity in gastric cancer. Oncoimmunology. 2018; 7:e1412900.
53. Wu S, et al., The Antitumor Effects of Vaccine-Activated CD8(+) T Cells Associate with Weak TCR Signaling and Induction of Stem-Like Memory T Cells. Cancer Immunol Res. 2017; 5:908-919.
54. Olson N C, et al., Decreased naive and increased memory CD4(+) T cells are associated with subclinical atherosclerosis: the multi-ethnic study of atherosclerosis. PLoS One. 2013; 8:e71498.
55. Olson N C, et al., Associations of Innate and Adaptive Immune Cell Subsets With Incident Type 2 Diabetes Risk: The MESA Study. J Clin Endocrinol Metab. 2020; 105.
56. Marsh M M, et al., Protection against atherosclerosis by estrogen is independent of plasma cholesterol levels in LDL receptor-deficient mice. J Lipid Res. 1999; 40:893-900.
57. Villablanca A C, Jayachandran M and Banka C. Atherosclerosis and sex hormones: current concepts.
Clin Sci (Lond). 2010; 119:493-513.
58. Gaddis D E, Padgett L E, Wu R and Hedrick C C. Neuropilin-1 Expression on CD4 T Cells Is Atherogenic and Facilitates T Cell Migration to the Aorta in Atherosclerosis. J Immunol. 2019; 203:3237-3246.
59. Gaddis D E, et al., Apolipoprotein AI prevents regulatory to follicular helper T cell switching during atherosclerosis. Nat Commun. 2018; 9:1095.
60. Taylor H A, Jr. The Jackson Heart Study: an overview. Ethn Dis. 2005; 15:S6-3.
61. Taylor H A, Jr., Wilson J G, Jones D W, Sarpong D F, Srinivasan A, Garrison R J, Nelson C and Wyatt S B. Toward resolution of cardiovascular health disparities in African Americans: design and methods of the Jackson Heart Study. Ethn Dis. 2005; 15:56-4-17.
62. Daugherty A, et al., American Heart Association Council on Arteriosclerosis T, Vascular B and Council on Basic Cardiovascular S. Recommendation on Design, Execution, and Reporting of Animal Atherosclerosis Studies: A Scientific Statement From the American Heart Association. Arterioscler Thromb Vasc Biol. 2017; 37:e131-e157.
63. Bruggner R V, et al., Automated identification of stratifying signatures in cellular subpopulations. Proc Natl Acad Sci USA. 2014; 111:E2770-7.
64. Hengst J, et al., High-resolution determination of human immune cell signatures from fine-needle liver aspirates. Eur J Immunol. 2015; 45:2154-7.
65. Ben-Shaanan T L, et al., Activation of the reward system boosts innate and adaptive immunity. Nat Med. 2016; 22:940-4.
66. Lau A H, et al., Mass cytometry reveals a distinct immunoprofile of operational tolerance in pediatric liver transplantation. Pediatr Transplant. 2016; 20:1072-1080.
67. Berben L, et al., Age-related remodelling of the blood immunological portrait and the local tumor immune response in patients with luminal breast cancer. Clin Transl Immunology. 2020; 9:e1184.
68. Stuart T, et al., Comprehensive Integration of Single-Cell Data. Cell. 2019; 177:1888-1902 e21.
69. Zhu Y P, et al., Identification of an Early Unipotent Neutrophil Progenitor with Pro-tumoral Activity in Mouse and Human Bone Marrow. Cell Rep. 2018; 24:2329-2341 e8.
70. Soneson C and Robinson M D. Bias, robustness and scalability in single-cell differential expression analysis. Nat Methods. 2018; 15:255-261.
71. Subramanian A, et al., Gene set enrichment analysis: a knowledge-based approach for interpreting genome-wide expression profiles. Proc Natl Acad Sci USA. 2005; 102:15545-50.
72. Aibar S, et al., SCENIC: single-cell regulatory network inference and clustering. Nat Methods. 2017; 14:1083-1086.
73. Lugli E, et al., Identification, isolation and in vitro expansion of human and nonhuman primate T stem cell memory cells. Nat Protoc. 2013; 8:33-42.
74. Gautam S, et al., The transcription factor c-Myb regulates CD8(+) T cell stemness and antitumor immunity. Nat Immunol. 2019; 20:337-349.
75. Hamers A A J, et al., Human Monocyte Heterogeneity as Revealed by High-Dimensional Mass Cytometry. Arterioscler Thromb Vase Biol. 2019; 39:25-36.
76. Thomas G D, et al., Human Blood Monocyte Subsets: A New Gating Strategy Defined Using Cell Surface Markers Identified by Mass Cytometry. Arterioscler Thromb Vase Biol. 2017; 37:1548-1558.
77. Bengsch B, et al., Epigenomic-Guided Mass Cytometry Profiling Reveals Disease-Specific Features of Exhausted CD8 T Cells. Immunity. 2018; 48:1029-1045 e5.
78. Winkler F and Bengsch B. Use of Mass Cytometry to Profile Human T Cell Exhaustion. Front Immunol. 2019; 10:3039.
79. Weiner D E, et al., Kidney disease as a risk factor for recurrent cardiovascular disease and mortality. Am J Kidney Dis. 2004; 44:198-206.
80. Rivellese A A, Riccardi G and Vaccaro O. Cardiovascular risk in women with diabetes.

Nutr Metab Cardiovasc Dis. 2010; 20:474-80.
81. Bang D, Xu J, et al., Cardiovascular Disease Prevalence in Patients with Osteoarthritis, Gout, or Both. Bull Hosp Jt Dis (2013). 2016; 74:113-8.
82. Zander N, et al., Dermatological comorbidity in psoriasis: results from a large-scale cohort of employees. Arch Dermatol Res. 2017; 309:349-356.
83. Boddupalli C S, et al., ABC transporters and NR4A1 identify a quiescent subset of tissue-resident memory T cells. J Clin Invest. 2016; 126:3905-3916.
84. Nowyhed H N, et al., ATP Binding Cassette Transporter ABCA7 Regulates NKT Cell Development and Function by Controlling CD1d Expression and Lipid Raft Content. Sci Rep. 2017; 7:40273.
85. Nowyhed H N, et al., Cutting Edge: The Orphan Nuclear Receptor Nr4a1 Regulates CD8+ T Cell Expansion and Effector Function through Direct Repression of Irf4. J Immunol. 2015; 195:3515-9.
86. Fontenot J D, Gavin M A and Rudensky A Y. Foxp3 programs the development and function of CD4+CD25+ regulatory T cells. Nat Immunol. 2003; 4:330-6.
87. Ramsdell F and Rudensky A Y. Foxp3: a genetic foundation for regulatory T cell differentiation and function. Nat Immunol. 2020; 21:708-709.
88. Rudensky A Y. Regulatory T cells and Foxp3. Immunol Rev. 2011; 241:260-8.
89. Robinson D S and O'Garra A. Further checkpoints in Th1 development. Immunity. 2002; 16:755-8.
90. Mullen A C, et al., Role of T-bet in commitment of TH1 cells before IL-12-dependent selection. Science. 2001; 292:1907-10.
91. Li J, He Y, et al., High Levels of Eomes Promote Exhaustion of Anti-tumor CD8(+) T Cells. Front Immunol. 2018; 9:2981.
92. Hoffmann J and Spyridopoulos I. Senescent cytotoxic T cells in acute myocardial infarction: innocent bystanders or the horsemen of apocalypse? Cell Mol Immunol. 2015; 12:510-2.
93. Zhang Y, et al., Host-reactive CD8⁺ memory stem cells in graft-versus-host disease.

Nat Med. 2005; 11:1299-305.
94. Wilhelm A J, et al., Dysregulated CD4+ T cells from SLE-susceptible mice are sufficient to accelerate atherosclerosis in LDLr−/− mice. Ann Rheum Dis. 2015; 74:778-85.
95. Lichtman A H, et al., Hyperlipidemia and atherosclerotic lesion development in LDL receptor-deficient mice fed defined semipurified diets with and without cholate. Arterioscler Thromb Vasc Biol. 1999; 19:1938-44.
96. Mottet C, Uhlig H H and Powrie F. Cutting edge: cure of colitis by CD4+CD25+ regulatory T cells. J Immunol. 2003; 170:3939-43.
97. Han S, et al., Macrophage insulin receptor deficiency increases ER stress-induced apoptosis and necrotic core formation in advanced atherosclerotic lesions. Cell Metab. 2006; 3:257-66.
98. Tabas I. Apoptosis and plaque destabilization in atherosclerosis: the role of macrophage apoptosis induced by cholesterol. Cell Death Differ. 2004; 11 Suppl 1:S12-6.
99. Hanna R N, et al., NR4A1 (Nur77) deletion polarizes macrophages toward an inflammatory phenotype and increases atherosclerosis. Circ Res. 2012; 110:416-27.
100. Fung-Leung et al., CD8 is needed for development of cytotoxic T cells but not helper T cells. Cell. 1991; 65:443-9.
101. Roche-Molina M, et al., Induction of sustained hypercholesterolemia by single adeno-associated virus-mediated gene transfer of mutant hPCSK9. Arterioscler Thromb Vasc Biol. 2015; 35:50-9.
102. Harrington L E, et al., Interleukin 17-producing CD4⁺ effector T cells develop via a lineage distinct from the T helper type 1 and 2 lineages. Nat Immunol. 2005; 6:1123-32.
103. Bending D, et al., Highly purified Th17 cells from BDC2.5NOD mice convert into Th1-like cells in NOD/SCID recipient mice. J Clin Invest. 2009; 119:565-72.

1. A method of diagnosing and treating a cardiovascular disease in a patient, the method comprising the steps of:
determining whether the patient has an increase in stem cell memory T cells (T_{scm}) by: obtaining or having obtained a biological sample from the patient;
performing or having performed an assay on the biological sample to determine the amount of T_{scm} in the patient, wherein the T_{scm} are at least one of:
T_{scm} CD8+CD45RA+CCR7+CD27+CD28+CD95+ T cells, T_{scm} CD4+CD45RA+CCR7+CD27+CD28+CD95+ T cells, or T_{scm} CD8+CD3+CD45RA+CCR7+CD45RO−CD95+ T cells,
identifying that the patient has an increase in T_{scm} when compared to the amount of T_{scm} in a healthy patient or when compared to the amount of T_{scm} in a low-cardiovascular disease (CVDlo) patient; and
if the patient has an increase in T_{scm}, then administering therapy for the treatment of the cardiovascular disease to the patient.

2. (canceled)

3. The method of claim 1, wherein the increase in T_{scm} when compared to the amount of T_{scm} in a healthy patient comprises determining the ratio of T_{scm} to naïve T (T_{N}) cells in the patient when compared to the ratio of T_{scm} to Tₙ cells in the healthy patient, wherein the T_{N} are CD8+CD3+CD45RA+CCR7+CD45RO−CD95− T cells, and wherein a higher ratio of T_{scm} to T_{N} in the patient indicates the patient has a greater risk of cardiovascular disease or more advanced cardiovascular disease; or
wherein a ratio of 1.5:1 T_{scm} to Tₙ cells or greater compared to the ratio of T_{scm} to Tₙ cells in the healthy patient indicates the patient has a cardiovascular disease;
wherein the amount of T_{scm} is determined by detecting the amount of T_{scm} in a blood sample, wherein the blood sample comprises peripheral blood mononuclear cells (PBMCs);
wherein the amounts of T_{scm} and Tn are determined by detecting the amounts T_{scm} and Tn in a blood sample;
wherein T_{scm} or the T_{N} express one or more of the following markers: CD127, CD11a, CD58, IL-2Rβ, or Bcl-2; or
wherein the increase of T_{scm} in the patient is further determined by measuring an increased expression of IFN-γ in T_{scm} of the patient compared to the expression of IFN-γ in T_{scm} of a healthy patient.

4.-9. (canceled)

10. The method of claim 1, wherein the T_{scm} express one or more of the following markers: CXCR3, CD122, LFA-1, c-Myb, Ki67, CD127, CD11a, CD58, IL-2Rβ, or Bcl-2.

11. (canceled)

12. The method of claim 1, wherein the therapy for the treatment of the cardiovascular disease comprises a T_{scm}, T_{N}, or T_{CD25} modulating therapy;
wherein the T_{scm}, T_{N}, or T_{CD25} modulating therapy is a T_{scm} depleting therapy, a T_{N} increasing therapy or a T_{CD25} CD8+CD3+CD25+ T cell therapy; or
wherein the therapy for the treatment of the cardiovascular disease is administered systemically, regionally or locally, via ingestion, via inhalation, topically, intravenously, or orally.

13.-15. (canceled)

16. The method of claim 1, wherein the diagnosing comprises determining the amount of increase in T_{scm} when compared to the baseline level of T_{scm} in a healthy patient comprises determining the ratio of T_{scm} to naïve T (T_{N}) cells in the patient when compared to the ratio of T_{scm} to T_{N} cells in the healthy patient, wherein the T_{N} are CD8+CD45RA+CCR7+CD27+CD28+ T cells, CD4+CD45RA+CCR7+CD27+CD28+ T cells, or T_{scm} CD8+CD3+CD45RA+CCR7+CD45RO−CD95+ T cells, and wherein at least one of: a higher ratio of T_{scm} to T_{N} in the patient indicates the patient has cardiovascular disease, is at risk of developing a cardiovascular disease, or has advanced cardiovascular disease; a patient with a high risk of myocardial infarction or heart attack (high CVD) has an increase in CD8+ T_{SCM} frequency (% of CD8+ T_{N} cells that are CD8+ T_{SCM} cells) when compared to a patient with a low risk of myocardial infarction or heart attack (low CVD); a patient with a high risk of myocardial infarction or heart attack (high CVD) has an increase of CD8+ T_{SCM} compared to subjects with a low risk of myocardial infarction or heart attack (low CVD); a patient with a high risk of myocardial infarction or heart attack (high CVD) have a decrease in CD8+ T_{N} compared to subjects with a low risk of myocardial infarction or heart attack (low CVD); CD8+ T_{SCM} from subjects with a high risk of myocardial infarction or heart attack (high CVD) have higher expression and release of pro-atherogenic cytokine IFN-γ compared to CD8+ T_{SCM} from subjects with low CVD; or CD8+ T_{SCM} from patients with a high risk of myocardial infarction or heart attack (high CVD) have a higher expression of Ki67 compared to CD8+ T_{scm} from subjects with low CVD.

17.-28. (canceled)

29. A method of determining if a patient has stem cell memory T (T_{scm}) cells that will increase the risk of developing a cardiovascular disease or the severity of an existing cardiovascular disease, comprising:
obtaining a biological sample from a patient; and
detecting the T_{scm} in the biological sample by contacting the biological sample with an agent that detects T cells expressing CD45RA+CCR7+CD27+CD28+CD95+, or CD45RA+CCR7+CD45RO−CD95+, and
determining whether T_{scm} are increased in the biological sample when compared to a baseline level of T_{scm} in a healthy patient, wherein an increase in T_{scm} in the biological sample compared to a healthy patient indicates the patient has cardiovascular disease, is at risk of developing a cardiovascular disease, or has advanced cardiovascular disease.

30. The method of claim 29, wherein the increase in T_{scm} when compared to the baseline level of T_{scm} in a healthy patient comprises determining the ratio of T_{scm} to naïve T (T_{N}) cells in the patient when compared to the ratio of T_{scm} to Tₙ cells in the healthy patient, wherein the T_{N} are CD8+CD45RA+CCR7+CD27+CD28+ T cells, CD4+CD45RA+CCR7+CD27+CD28+ T cells, or T_{scm} CD8+CD3+CD45RA+CCR7+CD45RO−CD95+ T cells, and wherein at least one of: a higher ratio of T_{scm} to T_{N} in the patient indicates the patient has cardiovascular disease, is at risk of developing a cardiovascular disease, or has advanced cardiovascular disease; a patient with a high risk of myocardial infarction or heart attack (high CVD) has an increase in CD8+ T_{SCM} frequency (% of CD8+ T_{N} cells that are CD8+T_{SCM} cells) when compared to a patient with a low risk of myocardial infarction or heart attack (low CVD); a patient with a high risk of myocardial infarction or heart attack (high CVD) has an increase of CD8+ T_{SCM} compared to subjects with a low risk of myocardial infarction or heart attack (low CVD); a patient with a high risk of myocardial infarction or heart attack (high CVD) have a decrease in CD8+ T_{N} compared to subjects with a low risk of myocardial infarction or heart attack (low CVD); CD8+ T_{SCM} from subjects with a high risk of myocardial infarction or heart attack (high CVD) have higher expression and release of pro-atherogenic cytokine IFN-γ compared to CD8+ T_{SCM} from subjects with low CVD; or CD8+ T_{SCM} from patients with a high risk of myocardial infarction or heart attack (high CVD) have a higher expression of Ki67 compared to CD8+ T_{scm} from subjects with low CVD; or
wherein a ratio of 1.5:1 T_{scm} to T₁ cells or greater compared to the ratio of T_{scm} to Tₙ cells in the healthy patient indicates the patient is at risk of developing a cardiovascular disease or having a cardiovascular disease of increased severity.

31.-36. (canceled)

37. The method of claim 29, wherein the T_{scm} are at least one of CD8+CD45RA+CCR7+CD27+CD28+CD95+ T cells, CD4+CD45RA+CCR7+CD27+CD28+CD95+ T cells, or CD8+CD3+CD45RA+CCR7+CD45RO−CD95+ T cells;
wherein the T_{scm} express one or more of the following markers: CXCR3, CD122, LFA-1, c-Myb, Ki67, CD127, CD11a, CD58, IL-2Rβ, or Bcl-2; or
wherein the increase of T_{scm} in the patient is further determined by measuring an increased expression of IFN-γ in T_{scm} of the patient compared to the expression of IFN-γ in T_{scm} of a healthy patient.

38.-49. (canceled)

50. The method of claim 1, wherein the increase of T_{scm} in the patient is further determined by measuring an increased expression of IFN-γ in T_{scm} of the patient compared to the expression of IFN-γ in T_{scm} of a CVDlo patient, or T_{scm} of a subject with high risk of myocardial infarction or heart attack (high CVD) express higher levels of Ki67 and IFN-γ than T_{scm} of a subject with low risk of myocardial infarction or heart attack (low CVD).

51.-53. (canceled)

54. A method for treating a patient suffering from advanced or severe cardiovascular disease, the method comprising the steps of:
determining whether the patient has an increase in stem cell memory T (T_{scm}) cells, when compared to a baseline level of T_{scm} present in a low-cardiovascular disease (CVDlo) patient by:
obtaining or having obtained a biological sample from the patient; and
performing or having performed an assay on the biological sample to determine if the patient has an increase in T_{scm} when compared to a baseline level of T_{scm} present in CVDlo patient; and
if the patient has an increase in T_{scm} when compared to a baseline level of T_{scm} present in a CVDlo patient, then administering therapy for the treatment of the cardiovascular disease to the patient.

55. The method of claim 54, wherein the increase in T_{scm} when compared to the baseline level of T_{scm} in a healthy patient comprises determining the ratio of T_{scm} to naïve T (T_{N}) cells in the patient when compared to the ratio of T_{scm} to Tₙ cells in the healthy patient, wherein the T_{N} are CD8+CD45RA+CCR7+CD27+CD28+ T cells, CD4+CD45RA+CCR7+CD27+CD28+ T cells, or T_{scm} CD8+CD3+CD45RA+CCR7+CD45RO−CD95+ T cells, and wherein at least one of: a higher ratio of T_{scm} to T_{N} in the patient indicates the patient has cardiovascular disease, is at risk of developing a cardiovascular disease, or has advanced cardiovascular disease; a patient with a high risk of myocardial infarction or heart attack (high CVD) has an increase in CD8+ T_{SCM} frequency (% of CD8+ T_{N} cells that are CD8+ T_{SCM} cells) when compared to a patient with a low risk of myocardial infarction or heart attack (low CVD); a patient with a high risk of myocardial infarction or heart attack (high CVD) has an increase of CD8+ T_{SCM} compared to subjects with a low risk of myocardial infarction or heart attack (low CVD); a patient with a high risk of myocardial infarction or heart attack (high CVD) have a decrease in CD8+ T_{N} compared to subjects with a low risk of myocardial infarction or heart attack (low CVD); CD8+ T_{SCM} from subjects with a high risk of myocardial infarction or heart attack (high CVD) have higher expression and release of pro-atherogenic cytokine IFN-γ compared to CD8+ T_{scm} from subjects with low CVD; or CD8+ T_{scm} from patients with a high risk of myocardial infarction or heart attack (high CVD) have a higher expression of Ki67 compared to CD8+ T_{scm} from subjects with low CVD.

56. The method of claim 55, wherein a ratio of 1.5:1 T_{scm} to T_{N} cells or greater compared to the ratio of T_{scm} to T_{N} cells in the CVDlo patient indicates the patient should be treated for the cardiovascular disease: wherein the amount of T_{scm} is determined by detecting the amount of T_{scm} in a blood sample, wherein the blood sample comprises peripheral blood mononuclear cells (PBMCs);
wherein the amounts of T_{scm} and Tn are determined by detecting the amounts T_{scm} and Tn in a blood sample;
wherein T_{scm} or the T_{N} express one or more of the following markers: CD127, CD11a, CD58, IL-2Rβ, or Bcl-2; or
wherein the increase of T_{scm} in the patient is further determined by measuring an increased expression of IFN-γ in T_{scm} of the patient compared to the expression of IFN-γ in T_{scm} of a healthy patient.

57. The method of claim 54, wherein the increase in T_{scm} is determined by detecting T_{scm} in a blood sample;
wherein the ratio of T_{scm} and T_{N} is determined by detecting T_{scm} and T_{N} in a blood sample;
wherein the T_{N} express one or more of the following markers: CD127, CD11a, CD58, IL-2Rβ, or Bcl-2; or
wherein the blood sample comprises peripheral blood mononuclear cells (PBMCs).

58.-63. (canceled)

64. The method of claim 54, wherein the increase of T_{scm} in the patient is further determined by measuring an increased expression of IFN-gamma in T_{scm} of the patient compared to the expression of IFN-gamma in T_{scm} of a CVDlo patient, or T_{scm} of a subject with high risk of myocardial infarction or heart attack (high CVD) express higher levels of Ki67 and IFN-γ than T_{scm} of a subject with low risk of myocardial infarction or heart attack (low CVD).

65.-78. (canceled)

79. The method of claim 29, wherein the agent is an antibody, a small molecule, a protein, a peptide, a ligand mimetic or a nucleic acid.

80.-91. (canceled)

92. The method of claim 1, further comprising
determining whether the patient has a higher ratio of stem cell memory T (T_{scm}) cells to naïve T (T_{N}) cells, compared to the ratio of T_{scm} to T_{N} cells in a healthy patient or a low-cardiovascular disease (CVDlo) patient, by:
obtaining or having obtained a biological sample from the patient; and
performing or having performed an assay on the biological sample to determine the ratio of T_{scm} to Tₙ cells in the patient when compared to the ratio of T_{scm} to T_{N} cells in a healthy patient or a low-cardiovascular disease (CVDlo) patient; and
if the patient has a higher ratio of T_{scm} to T_{N} than the ratio of T_{scm} to T_{N} in the healthy patient or CVDlo patient, administering therapy for the treatment of the cardiovascular disease to the patient.

93. (canceled)

94. The method of claim 92, wherein a ratio of 1.5:1 T_{scm} to T_{N} cells or greater compared to the ratio of T_{scm} to T_{N} cells in a healthy patient or a low-cardiovascular disease (CVDlo) patient indicates the patient should be treated for the cardiovascular disease;
wherein the ratio of T_{scm} and T_{N} are determined by detecting T_{scm} and T_{N} in a blood sample;
wherein the blood sample comprises peripheral blood mononuclear cells (PBMCs);
wherein the Ta express one or more of the following markers: CD127, CD11a, CD58, IL-2Rβ, or Bcl-2.

95-103. (canceled)

104. The method of claim 1, further comprising determining if a patient has stem cell memory T (T_{scm}) cells that will increase the risk of developing a cardiovascular disease or the severity of an existing cardiovascular disease, comprising:
obtaining a biological sample from a patient; and
detecting the ratio of T_{scm} to naïve T (T_{N}) cells in the biological sample by contacting the biological sample with an agent that detects T cells expressing CD45RA+CCR7+CD27+CD28+CD95+, CD45RA+CCR7+CD27+CD28+CD95−/CD45RA+CCR7+CD27+CD28+CD95lo, or CD45RA+CCR7+CD45RO−CD95+ and determining the ratio of T_{scm} to Tₙ in the biological sample when compared to the ratio of T_{scm} to T_{N} cells in a healthy patient or a low-cardiovascular disease (CVDlo) patient, wherein a higher ratio of T_{scm} to T_{N} in the biological sample compared to the ratio of T_{scm} to T_{N} in the healthy patient or CVDlo patient indicates the patient has cardiovascular disease, is at risk of developing a cardiovascular disease, or has advanced cardiovascular disease.

105.-113. (canceled)

114. The method of claim 1, further comprising
determining a ratio of T_{scm} to T_{N} from a biological sample obtained from the patient, wherein a higher ratio of T_{scm} to T_{N} in the biological sample compared to the ratio of T_{scm} to T_{N} in a healthy patient or CVDlo patient indicates the patient is in need of treatment to prevent or treat the cardiovascular disease; and
treating the patient with a T_{scm}, T_{N}, or T_{CD25} modulating therapy.

115. The method of claim 114, wherein the T_{scm} or T_{N} modulating therapy is a T_{scm} depleting therapy, a T_{N} increasing therapy, or a T_{CD25} CD8+CD3+CD25+ T cell therapy.

116. The method of claim 114, wherein the therapy for the treatment of the cardiovascular disease is administered systemically, regionally or locally, via ingestion, via inhalation, topically, intravenously, or orally.

117. The method of claim 114, wherein the therapy for the treatment of the cardiovascular disease is adoptive T cell therapy, chimeric antigen receptor T cell (CAR-T) therapy, antibody dependent cell cytotoxicity therapy, antibody drug conjugate therapy, T-cell receptor (TCR) modified T-cell therapy, chimeric antigen receptor (CAR)-modified natural killer cell therapy, administration of monoclonal antibodies, administration of cytokines, T cell suppressing therapy, or any combination thereof.
